(19) United States
(12) Patent Application Publication
(10) Pub. No.: US 2023/0190743 A1
(43) Pub. Date: Jun. 22, 2023

(54) TREATMENT WITH ILEAL BILE ACID TRANSPORTER (IBAT) INHIBITORS FOR INCREASED EVENT-FREE SURVIVAL (EFS)

(71) Applicant: Mirum Pharmaceuticals, Inc., Foster City, CA (US)

(72) Inventors: Pamela VIG, Foster City, CA (US); Will Garner, Foster City, CA (US)

(73) Assignee: Mirum Pharmaceuticals, Inc., Foster City, CA (US)

(21) Appl. No.: 17/981,033

(22) Filed: Nov. 4, 2022

Related U.S. Application Data
(60) Provisional application No. 63/315,762, filed on Mar. 2, 2022.
(60) Provisional application No. 63/276,480, filed on Nov. 5, 2021.

(51) Int. Cl. A61K 31/4995; A61K 31/38; A61P 1/16; G16H 20/10

(52) U.S. Cl. CPC A61K 31/4995; A61K 31/38; A61P 1/16; G16H 20/10

(57) ABSTRACT

The present invention relates generally to methods treating cholestatic liver disease by administering an ileal bile acid transporter inhibitor (IBAT inhibitor), wherein the treatment results in increased event-free survival (EFS). The present invention relates also to methods for providing a prediction of response to an IBAT inhibitor therapy for treatment of cholestatic liver disease by predicting EFS.

### CROSS-REFERENCE TO RELATED APPLICATIONS

This application claims priority to U.S. Provisional Application Nos. 63/315,762, filed Mar. 2, 2022, and 63/276,480, filed Nov. 5, 2021, the disclosures of which are herein incorporated by reference in their entirety.

### FIELD OF THE INVENTION

The present invention relates generally to methods of treating cholestatic liver disease by administering an ileal bile acid transporter (IBAT) inhibitor, wherein the treatment results in increased event-free survival (EFS). The present invention relates also to methods for providing a prediction of response to an IBAT inhibitor therapy for treatment of cholestatic liver disease by predicting EFS.

### BACKGROUND

Hypercholemia and cholestatic liver diseases are liver diseases associated with impaired bile secretion (i.e., cholestasis), associated with and often secondary to the intracellular accumulation of bile acids/salts in the hepatocyte. Hypercholemia is characterized by increased serum concentration of bile acid or bile salt. Cholestasis can be categorized clinicopathologically into two principal categories of obstructive, often extrahepatic, cholestasis, and nonobstructive, or intrahepatic, cholestasis. Nonobstructive intrahepatic cholestasis can further be classified into two principal subgroups of primary intrahepatic cholestasis that result from constitutively defective bile secretion, and secondary intrahepatic cholestasis that result from hepatocellular injury. Primary intrahepatic cholestasis includes diseases such as benign recurrent intrahepatic cholestasis, which is predominantly an adult form with similar clinical symptoms, and progressive familial intrahepatic cholestasis (PFIC) types 1, 2, and 3, which are diseases that affect children.

Pediatric cholestatic liver diseases affect a small percentage of children, but therapy results in significant healthcare costs each year. Currently, many of the pediatric cholestatic liver diseases require invasive and costly treatments such as liver transplantation and surgery. There is a need for an effective, less invasive, transplant-free survival (TFS) treatment that provides long-term event-free survival (EFS).

### SUMMARY OF THE INVENTION

Various non-limiting aspects and embodiments of the invention are described below.

In one aspect, the present invention provides a method of treating cholestatic liver disease in a subject in need thereof, the method comprising administering to the subject a therapeutically effective amount of an ileal bile acid transporter (IBAT) inhibitor, wherein the treatment increases event-free survival (EFS) of the subject by reducing one or more of:

a) total bilirubin (TB);
b) total serum bile acids (sBA), and
c) pruritus score as measured by an Itch Reported Outcome (ItchRO) severity assessment tool.

In one embodiment, the TB is reduced to about 6.5 mg/dL or below.

In one embodiment, the sBA level is reduced to about 200 μmol/L or below.

In one embodiment, the pruritus ItchRO score is reduced by at least about 1 point as compared to the time of first administration of the IBAT inhibitor.

In one embodiment, the TB, the sBA, or the pruritus score is determined 18 weeks after initiation of the IBAT inhibitor treatment. In one embodiment, the TB, the sBA, or the pruritus score is determined 24 weeks after initiation of the IBAT inhibitor treatment. In one embodiment, the TB, the sBA, or the pruritus score is determined 48 weeks after initiation of the IBAT inhibitor treatment.

In one embodiment, the TB, the sBA, or the pruritus score is reduced as compared to the time of first administration of the IBAT inhibitor.

In one aspect, the present invention provides a method for providing a prediction of response to an IBAT inhibitor therapy for treatment of cholestatic liver disease in a subject in need thereof by predicting event-free survival (EFS), the method comprising obtaining one or more of total bilirubin (TB) data, total serum bile acids (sBA) data, pruritus reduction data and age of the subject at initiation of treatment with the IBAT inhibitor, and using the data obtained for the subject to predict EFS.

In one embodiment, the EFS is predicted when the TB is less than about 6.5 mg/dL.

In one embodiment, the TB is determined 48 weeks after initiation of the IBAT inhibitor treatment.

In one embodiment, the EFS is predicted when the sBA level after treatment with the IBAT inhibitor is less than about 200 μmol/L.

In one embodiment, the sBA level is determined 18 weeks after initiation of the IBAT inhibitor treatment. In one embodiment, the sBA level is determined 24 weeks after initiation of the IBAT inhibitor treatment. In one embodiment, the sBA level is determined 48 weeks after initiation of the IBAT inhibitor treatment.

In one embodiment, the EFS is predicted when the pruritus reduction is at least about 1 point after treatment with the IBAT inhibitor compared to the pruritus at the time of first administration of the IBAT inhibitor, wherein the pruritus is measured by an Itch Reported Outcome (ItchRO) severity assessment tool.

In one embodiment, the pruritus is determined 18 weeks after the initiation of the IBAT inhibitor treatment. In one embodiment, the pruritus is determined 24 weeks after the initiation of the IBAT inhibitor treatment. In one embodiment, the pruritus is determined 48 weeks after the initiation of the IBAT inhibitor treatment.

In one embodiment, the EFS is predicted when the age of the subject at the time of initiation of treatment is equal to or higher than about 36 months.

In one embodiment, the EFS comprises survival in the absence of one or more of hepatic decompensation, surgical biliary diversion, liver transplantation or death.

In one embodiment, the EFS comprises survival of the subject in the absence of liver transplant.

In one embodiment, the treatment with the IBAT inhibitor further results in reduction of cholestatic pruritus.

In one embodiment, the administration is sufficient to result in event-free survival of the subject for at least 18 months following the first dose of the IBAT inhibitor. In one embodiment, the administration is sufficient to result in event-free survival of the subject for at least 2 years following the first dose of the IBAT inhibitor. In one embodiment, the administration is sufficient to result in event-free survival of the subject for 6 years following the first dose of the IBAT inhibitor.

In one embodiment, the cholestatic liver disease is a pediatric cholestatic liver disease.

In one embodiment, the cholestatic liver disease is an adult cholestatic liver disease.

In one embodiment, the cholestatic liver disease is non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy, contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome (ALGS), biliary atresia, post-Kasai biliary atresia, post-liver transplantation biliary atresia, post-liver transplantation cholestasis, post-liver transplantation associated liver disease, intestinal failure associated liver disease, bile acid mediated liver injury, MRP2 deficiency syndrome, or neonatal sclerosing cholangitis.

In one embodiment, the cholestatic liver disease is ALGS, PFIC, BRIC, PSC, PBC, or biliary atresia.

In one embodiment, the sBA comprise one or more of TCA, TUDCA, TCDCA, TDCA, TLCA, GCA, GUDCA, GCDCA, GDCA, GLCA, CA, UDCA, CDCA, DCA, and LCA.

In another aspect, the present invention provides a method of treating cholestatic liver disease with pruritus in a subject in need thereof, the method comprising administering to the subject a therapeutically effective amount of an IBAT inhibitor or a pharmaceutically acceptable salt thereof, wherein the administration increases event-free survival (EFS) of the subject for at least 18 months following the first dose of the IBAT inhibitor by reducing pruritus score as measured by an Itch Reported Outcome (ItchRO) severity assessment tool by at least about 1 point.

In one embodiment, the cholestatic liver disease with pruritus is selected from the group consisting of ALGS, PFIC, BRIC, PSC, PBC and biliary atresia.

In one embodiment, the pruritus score is determined 18 weeks after initiation of the IBAT inhibitor treatment. In one embodiment, the pruritus score is determined 24 weeks after initiation of the IBAT inhibitor treatment. In one embodiment, the pruritus score is determined 48 weeks after initiation of the IBAT inhibitor treatment.

In another aspect, the present invention provides a method of treating cholestatic liver disease with elevated total serum bile acids (sBA) in a subject in need thereof, the method comprising administering to the subject a therapeutically effective amount of an IBAT inhibitor or a pharmaceutically acceptable salt thereof, wherein the administration increases event-free survival (EFS) of the subject for at least 18 months following the first dose of the IBAT inhibitor by reducing sBA to about 200 μmol/L or below.

In one embodiment, the cholestatic liver disease with elevated sBA is selected from the group consisting of ALGS, PFIC, BRIC, PSC, PBC, and biliary atresia.

In one embodiment, the sBA is determined 18 weeks after initiation of the IBAT inhibitor treatment. In one embodiment, the sBA is determined 24 weeks after initiation of the IBAT inhibitor treatment. In one embodiment, the sBA is determined 48 weeks after initiation of the IBAT inhibitor treatment.

In one embodiment, the sBA comprise one or more of TCA, TUDCA, TCDCA, TDCA, TLCA, GCA, GUDCA, GCDCA, GDCA, GLCA, CA, UDCA, CDCA, DCA, and LCA.

In another embodiment, the present invention provides a method of treating cholestatic liver disease with elevated total bilirubin (TB) in a subject in need thereof, the method comprising administering to the subject a therapeutically effective amount of an IBAT inhibitor or a pharmaceutically acceptable salt thereof, wherein the administration increases event-free survival (EFS) of the subject for at least 18 months following the first dose of the IBAT inhibitor by reducing TB to about 6.5 mg/dL or below.

In one embodiment, the cholestatic liver disease with elevated TB is biliary atresia (BA).

In one embodiment, the TB is determined 48 weeks after initiation of the IBAT inhibitor treatment.

In one embodiment, the administration is sufficient to result in event-free survival of the subject for at least 2 years following the first dose of the IBAT inhibitor. In one embodiment, the administration is sufficient to result in event-free survival of the subject for 6 years following the first dose of the IBAT inhibitor.

In one embodiment, the IBAT inhibitor is administered once daily.

In one embodiment, the IBAT inhibitor is administered twice daily.

In one embodiment, the IBAT inhibitor is administered in an amount of about 0.1 mg to about 100 mg per dose. In one embodiment, the IBAT inhibitor is administered in an amount of about 10 mg to about 100 mg per dose. In one embodiment, the IBAT inhibitor is administered in an amount of about 20 mg to about 80 mg per dose.

In one embodiment, the IBAT inhibitor is administered in an amount of about 100 ug/kg/day to 1400 ug/kg/day. In one embodiment, the IBAT inhibitor is administered in an amount of about 400 ug/kg/day to about 800 ug/kg/day.

In one embodiment, the subject has a BSEP deficiency.

In one embodiment, the IBAT inhibitor is

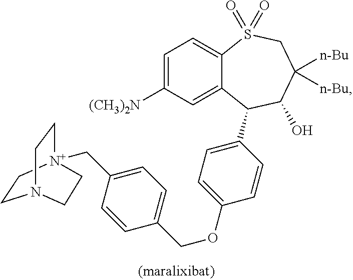

or a pharmaceutically acceptable salt thereof.

In one embodiment, the IBAT inhibitor is

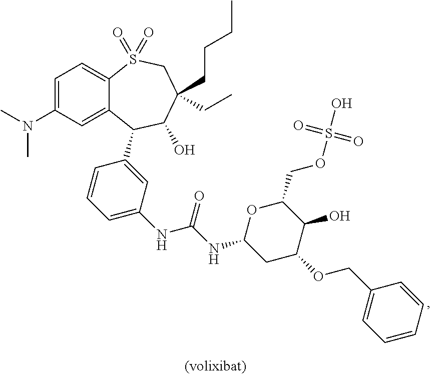 [SMILES: CCCC[C@]1(CC)CS(=O)(=O)c2ccc(N(C)C)cc2[C@@H](c2cccc(NC(=O)N[C@H]3C[C@@H](OCc4ccccc4)[C@H](O)[C@@H](COS(=O)(=O)O)O3)c2)[C@H]1O]

or a pharmaceutically acceptable salt thereof.

In one embodiment, the IBAT inhibitor is

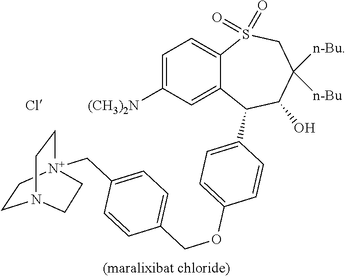

In one embodiment, the IBAT inhibitor is

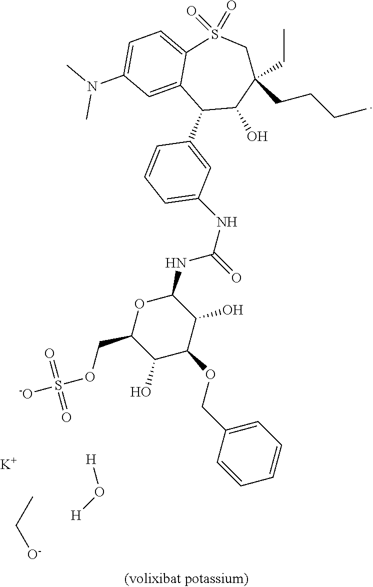

In one embodiment, the subject is a pediatric subject. In one embodiment, the pediatric subject is 0 to 18 years of age.

In one embodiment, the IBAT inhibitor is administered orally.

In one embodiment, less than 10% of the IBAT inhibitor is systemically absorbed. In one embodiment, less than 30% of the IBAT inhibitor is systemically absorbed.

In yet another aspect, the present invention provides a method of treating Alagille syndrome in a pediatric subject in need thereof, the method comprising administering to the subject a therapeutically effective amount of maralixibat or a pharmaceutically acceptable salt thereof, wherein the administration increases event-free survival (EFS) of the subject for at least 18 months following the first dose of maralixibat by reducing one or more of:

a) total bilirubin (TB) to about 6.5 mg/dL or below;
b) total serum bile acids (sBA) to about 200 μmol/L or below, and
c) pruritus score as measured by an Itch Reported Outcome (ItchRO) severity assessment tool by at least about 1 point.

In one embodiment, the treatment increases liver transplant-free survival (TFS) for at least 18 months following the first dose of maralixibat.

In yet another aspect, the present invention provides a method for providing a prediction of response to maralixibat therapy for treatment of Alagille syndrome in a subject in need thereof by predicting event-free survival (EFS) for 6 years following the first dose of the maralixibat, the method comprising: obtaining one or more of total bilirubin (TB) data, total serum bile acids (sBA) data, pruritus reduction data and age of the subject at initiation of treatment with maralixibat, and using the data obtained for the subject to predict the EFS.

In one embodiment, the maralixibat is maralixibat chloride.

In one embodiment, the sBA comprise one or more of TCA, TUDCA, TCDCA, TDCA, TLCA, GCA, GUDCA, GCDCA, GDCA, GLCA, CA, UDCA, CDCA, DCA, and LCA.

These and other aspects of the present invention will become apparent to those skilled in the art after a reading of the following detailed description of the invention, including the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a Kaplan-Meier Plot for Event-Free Survival in Maralixibat Cohort versus GALA Control Group.

FIGS. 2A-2D shows Kaplan-Meier plots of EFS according to the following variables: (FIG. 2A) week 48 total bilirubin, (FIG. 2B) week 48 sBA, (FIG. 2C) change from baseline to week 48 ItchRO(Obs), and (FIG. 2D) age at initiation of maralixibat. Data values under each panel indicate the number of patients at each time point. EFS, event-free survival; ItchRO(Obs), itch-reported outcome (observer); pt, point; sBA, serum bile acid.

FIGS. 3A-3C depict HRQoL scores at baseline and week 48 according to ItchRO response status. PedsQL Generic Core Total Scale Score is shown in FIG. 3A, Family Impact Total Scale Score is shown in FIG. 3B and Multidimensional Fatigue Total Scale Score is shown in FIG. 3C. Unfilled squares and green arrows represent the mean treatment response and HRQoL values at baseline and week 48 among all responders and non-responders. Individual changes from baseline (unfilled circles) to week 48 (filled circles) are shown for responders (pink circles and arrows) and non-responders (blue circles and arrows). All arrows are directional according to baseline and week 48.

FIGS. 4A-4C depict HRQoL scores at baseline and week 48 according to sBA response status. PedsQLGeneric Core Total Scale Score is shown in FIG. 4A, Multidimensional Fatigue Total Scale Score is shown in FIG. 4B, and Family Impact Total Scale Score is shown in FIG. 4C. Unfilled squares and green arrows represent the mean treatment response and HRQoL values at baseline and week 48 among all responders and non-responders. Individual changes from baseline to week 48 are shown for responders (pink dots and arrows) and non-responders (blue dots and arrows). All arrows are directional according to baseline and week 48.

FIG. 5 is a plot of patient disposition during the study. Abbreviations: AE, adverse event, BSEP, bile salt export pump; FIC, familial intrahepatic cholestasis; nt-BSEP, nontruncating BSEP; t-BSEP, truncating BSEP.

FIGS. 6A-6B depict Individual changes from Baseline to Week 240 in sBA levels in (A) sBA responders and (B) sBA non-responders. The black circle in FIG. 6A indicates when the seventh responder initiated twice-daily dosing at Week 97. Abbreviation: sBA, serum bile acid.

FIGS. 7A-7B show individual changes from Baseline to Week 240 in ItchRO(Obs) scores in (A) sBA responders and (B) sBA non-responders. Three non-responders achieved a change of >1.0 points in ItchRO(Obs), deemed clinically significant. Abbreviations: ItchRO(Obs), Itch Reported Outcome Observer; sBA, serum bile acids.

FIGS. 8A-8B plot mean changes in (A) height z-scores and (B) weight z-scores from Baseline to Week 240 in sBA responders and sBA non-responders. Abbreviations: sBA, serum bile acid; SE, standard error.

FIG. 9 shows changes in mean PedsQL™ scores from Baseline to Week 240 in sBA responders versus sBA non-responders (patients with BSEP deficiency, n=25). Abbreviations: PedsQL™, Pediatric Quality of Life Inventory™; sBA, serum bile acids; SE, standard error.

FIG. 10 shows changes throughout the study in ALT, AST and bilirubin (total and direct) in the 7 sBA responders. Abbreviations: ALT, alanine aminotransferase; AST, aspartate aminotransferase; U, units; L, liter.

FIG. 11 shows changes in individual 7α-C4/sBA ratios from Baseline to Week 240 in sBA responders and sBA non-responders (patients with BSEP deficiency; n=25). Abbreviations: sBA, serum bile acid; 7α-C4, 7α-hydroxy-4-cholesten-3-one.

FIG. 12 shows transplant-free survival in sBA responders and sBA non-responders. Abbreviation: sBA, serum bile.

acid

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Bile acids/salts play a critical role in activating digestive enzymes and solubilizing fats and fat-soluble vitamins and are involved in liver, biliary, and intestinal disease. Bile acids are synthesized in the liver by a multistep, multiorganelle pathway. Hydroxyl groups are added to specific sites on the steroid structure, the double bond of the cholesterol B ring is reduced, and the hydrocarbon chain is shortened by three carbon atoms resulting in a carboxyl group at the end of the chain. The most common bile acids are cholic acid and chenodeoxycholic acid (the “primary bile acids”). Before exiting the hepatocytes and forming bile, the bile acids are conjugated to either glycine (to produce glycocholic acid or glycochenodeoxycholic acid) or taurine (to produce taurocholic acid or taurochenodeoxycholic acid). The conjugated bile acids are called bile salts and their amphipathic nature makes them more efficient detergents than bile acids. Bile salts, not bile acids, are found in bile.

Bile salts are excreted by the hepatocytes into the canaliculi to form bile. The canaliculi drain into the right and left hepatic ducts and the bile flows to the gallbladder. Bile is released from the gallbladder and travels to the duodenum, where it contributes to the metabolism and degradation of fat. The bile salts are reabsorbed in the terminal ileum and transported back to the liver via the portal vein. Bile salts often undergo multiple enterohepatic circulations before being excreted via feces. A small percentage of bile salts may be reabsorbed in the proximal intestine by either passive or carrier-mediated transport processes. Most bile salts are reclaimed in the distal ileum by a sodium-dependent apically located bile acid transporter referred to as ileal bile acid transporter (IBAT). At the basolateral surface of the enterocyte, a truncated version of IBAT is involved in vectoral transfer of bile acids/salts into the portal circulation. Completion of the enterohepatic circulation occurs at the basolateral surface of the hepatocyte by a transport process that is primarily mediated by a sodium-dependent bile acid transporter. Intestinal bile acid transport plays a key role in the enterohepatic circulation of bile salts. Molecular analysis of this process has recently led to important advances in understanding of the biology, physiology and pathophysiology of intestinal bile acid transport.

Within the intestinal lumen, bile acid concentrations vary, with the bulk of the reuptake occurring in the distal intestine. Described herein are certain compositions and methods that control bile acid concentrations in the intestinal lumen, thereby controlling the hepatocellular damage caused by bile acid accumulation in the liver and dosing in the fasted state for minimal gastrointestinal adverse effects.

The presently disclosed subject matter is based, at least in part, on the surprising discovery that treatment of an IBAT inhibitor to a subject in need thereof leads to long-term event-free survival (EFS) in patients where reduction in one or more of a) total bilirubin levels, b) total serum bile acid levels, and c) pruritus score are met.

### Classes of Cholestasis and Cholestatic Liver Disease

As used herein, “cholestasis” means the disease or symptoms comprising impairment of bile formation and/or bile flow. As used herein, “cholestatic liver disease” means a liver disease associated with cholestasis. Cholestatic liver diseases are often associated with jaundice, fatigue, and pruritis. Biomarkers of cholestatic liver disease include elevated serum bile acid concentrations, elevated serum alkaline phosphatase (AP), elevated gamma-glutamyltranspeptidease, elevated conjugated hyperbilirubinemia, and elevated serum cholesterol.

Cholestatic liver disease can be sorted clinicopathologically between two principal categories of obstructive, often extrahepatic, cholestasis, and nonobstructive, or intrahepatic, cholestasis. In the former, cholestasis results when bile flow is mechanically blocked, as by gallstones or tumor, or as in extrahepatic biliary atresia.

The latter group who has nonobstructive intrahepatic cholestasis in turn fall into two principal subgroups. In the first subgroup, cholestasis results when processes of bile secretion and modification, or of synthesis of constituents of bile, are caught up secondarily in hepatocellular injury so severe that nonspecific impairment of many functions can be expected, including those subserving bile formation. In the second subgroup, no presumed cause of hepatocellular injury can be identified. Cholestasis in such patients appears to result when one of the steps in bile secretion or modification, or of synthesis of constituents of bile, is constitutively damaged. Such cholestasis is considered primary.

Accordingly, provided herein are methods and compositions for stimulating epithelial proliferation and/or regeneration of intestinal lining and/or enhancement of the adaptive processes in the intestine in individuals with hypercholemia and/or a cholestatic liver disease. In some of such embodiments, the methods comprise increasing bile acid concentrations and/or GLP-2 concentrations in the intestinal lumen.

Increased levels of bile acids, and elevated levels of AP (alkaline phosphatase), LAP (leukocyte alkaline phosphatase), gamma GT (gamma-glutamyl transpeptidase), and 5′-nucleotidase are biochemical hallmarks of cholestasis and cholestatic liver disease. Accordingly, provided herein are methods and compositions for stimulating epithelial proliferation and/or regeneration of intestinal lining and/or enhancement of the adaptive processes in the intestine in individuals with hypercholemia, and elevated levels of AP (alkaline phosphatase), LAP (leukocyte alkaline phosphatase), gamma GT (gamma-glutamyl transpeptidase or GGT), and/or 5′-nucleotidase. In some of such embodiments, the methods comprise increasing bile acid concentrations in the intestinal lumen. Further provided herein, are methods and compositions for reducing hypercholemia, and elevated levels of AP (alkaline phosphatase), LAP (leukocyte alkaline phosphatase), gamma GT (gamma-glutamyl transpeptidase), and 5′-nucleotidase comprising reducing overall serum bile acid load by excreting bile acid in the feces.

Pruritus is often associated with hypercholemia and cholestatic liver diseases. It has been suggested that pruritus results from bile salts acting on peripheral pain afferent nerves. The degree of pruritus varies with the individual (i.e., some individuals are more sensitive to elevated levels of bile acids/salts).

Administration of agents that reduce serum bile acid concentrations has been shown to reduce pruritus in certain individuals. Accordingly, provided herein are methods and compositions for stimulating epithelial proliferation and/or regeneration of intestinal lining and/or enhancement of the adaptive processes in the intestine in individuals with pruritus. In some of such embodiments, the methods comprise increasing bile acid concentrations in the intestinal lumen. Further provided herein, are methods and compositions for treating pruritus comprising reducing overall serum bile acid load by excreting bile acid in the feces.

Another symptom of hypercholemia and cholestatic liver disease is the increase in serum concentration of conjugated bilirubin. Elevated serum concentrations of conjugated bilirubin result in jaundice and dark urine. The magnitude of elevation is not diagnostically important as no relationship has been established between serum levels of conjugated bilirubin and the severity of hypercholemia and cholestatic liver disease. Conjugated bilirubin concentration rarely exceeds 30 mg/dL. Accordingly, provided herein are methods and compositions for stimulating epithelial proliferation and/or regeneration of intestinal lining and/or enhancement of the adaptive processes in the intestine in individuals with elevated serum concentrations of conjugated bilirubin. In some of such embodiments, the methods comprise increasing bile acid concentrations in the intestinal lumen. Further provided herein, are methods and compositions for treating elevated serum concentrations of conjugated bilirubin comprising reducing overall serum bile acid load by excreting bile acid in the feces.

Increased serum concentration of nonconjugated bilirubin is also considered diagnostic of hypercholemia and cholestatic liver disease. Portions of serum bilirubin and covalently bound to albumin (delta bilirubin or biliprotein). This fraction may account for a large proportion of total bilirubin in patients with cholestatic jaundice. The presence of large quantities of delta bilirubin indicates long-standing cholestasis. Delta bilirubin in cord blood or the blood of a newborn is indicative of cholestasis/cholestatic liver disease that antedates birth. Accordingly, provided herein are methods and compositions for stimulating epithelial proliferation and/or regeneration of intestinal lining and/or enhancement of the adaptive processes in the intestine in individuals with elevated serum concentrations of nonconjugated bilirubin or delta bilirubin. In some of such embodiments, the methods comprise increasing bile acid concentrations in the intestinal lumen. Further provided herein, are methods and compositions for treating elevated serum concentrations of nonconjugated bilirubin and delta bilirubin comprising reducing overall serum bile acid load by excreting bile acid in the feces.

Cholestasis and cholestatic liver disease results in hypercholemia. During metabolic cholestasis, the hepatocytes retains bile salts. Bile salts are regurgitated from the hepatocyte into the serum, which results in an increase in the concentration of bile salts in the peripheral circulation. Furthermore, the uptake of bile salts entering the liver in portal vein blood is inefficient, which results in spillage of bile salts into the peripheral circulation. Accordingly, provided herein are methods and compositions for stimulating epithelial proliferation and/or regeneration of intestinal lining and/or enhancement of the adaptive processes in the intestine in individuals with hypercholemia. In some of such embodiments, the methods comprise increasing bile acid concentrations in the intestinal lumen. Further provided herein, are methods and compositions for treating hypercholemia comprising reducing overall serum bile acid load by excreting bile acid in the feces.

Hyperlipidemia is characteristic of some but not all cholestatic diseases. Serum cholesterol is elevated in cholestasis due to the decrease in circulating bile salts which contribute to the metabolism and degradation of cholesterol. Cholesterol retention is associated with an increase in membrane cholesterol content and a reduction in membrane fluidity and membrane function. Furthermore, as bile salts are the metabolic products of cholesterol, the reduction in cholesterol metabolism results in a decrease in bile acid/salt synthesis. Serum cholesterol observed in children with cholestasis ranges between about 1,000 mg/dL and about 4,000 mg/dL. Accordingly, provided herein are methods and compositions for stimulating epithelial proliferation and/or regeneration of intestinal lining and/or enhancement of the adaptive processes in the intestine in individuals with hyperlipidemia. In some of such embodiments, the methods comprise increasing bile acid concentrations in the intestinal lumen. Further provided herein, are methods and compositions for treating hyperlipidemia comprising reducing overall serum bile acid load by excreting bile acid in the feces.

In individuals with hypercholemia and cholestatic liver diseases, xanthomas develop from the deposition of excess circulating cholesterol into the dermis. The development of xanthomas is more characteristic of obstructive cholestasis than of hepatocellular cholestasis. Planar xanthomas first occur around the eyes and then in the creases of the palms and soles, followed by the neck. Tuberous xanthomas are associated with chronic and long-term cholestasis. Accordingly, provided herein are methods and compositions for stimulating epithelial proliferation and/or regeneration of intestinal lining and/or enhancement of the adaptive processes in the intestine in individuals with xanthomas. In some of such embodiments, the methods comprise increasing bile acid concentrations in the intestinal lumen. Further provided herein, are methods and compositions for treating xanthomas comprising reducing overall serum bile acid load by excreting bile acid in the feces.

In children with chronic cholestasis, one of the major consequences of hypercholemia and cholestatic liver disease is failure to thrive. Failure to thrive is a consequence of reduced delivery of bile salts to the intestine, which contributes to inefficient digestion and absorption of fats, and reduced uptake of vitamins (vitamins E, D, K, and A are all malabsorbed in cholestasis). Furthermore, the delivery of fat into the colon can result in colonic secretion and diarrhea. Treatment of failure to thrive involves dietary substitution and supplementation with long-chain triglycerides, medium-chain triglycerides, and vitamins. Ursodeoxycholic acid, which is used to treat some cholestatic conditions, does not form mixed micelles and has no effect on fat absorption. Accordingly, provided herein are methods and compositions for stimulating epithelial proliferation and/or regeneration of intestinal lining and/or enhancement of the adaptive processes in the intestine in individuals (e.g., children) with failure to thrive. In some of such embodiments, the methods comprise increasing bile acid concentrations in the intestinal lumen. Further provided herein, are methods and compositions for treating failure to thrive comprising reducing overall serum bile acid load by excreting bile acid in the feces.

### Primary Biliary Cirrhosis (PBC)

Primary biliary cirrhosis is an autoimmune disease of the liver characterized by the destruction of the bile canaliculi. Damage to the bile cancliculi results in the build-up of bile in the liver (i.e., cholestasis). The retention of bile in the liver damages liver tissue and may lead to scarring, fibrosis, and cirrhosis. PBC usually presents in adulthood (e.g., ages 40 and over). Individuals with PBC often present with fatigue, pruritus, and/or jaundice. PBC is diagnosed if the individual has elevated AP concentrations for at least 6 months, elevated gammaGT levels, antimitochondrial antibodies (AMA) in the serum (>1:40), and florid bile duct lesions. Serum ALT and serum AST and conjugated bilirubin may also be elevated, but these are not considered diagnostic. Cholestasis associated with PBC has been treated or ameliorated by administration of ursodeoxycholic acid (UDCA or Ursodiol). Corticosteroids (e.g., prednisone and budesonide) and immunosuppressive agents (e.g., azathioprine, cyclosporin A, methotrexate, chlorambucil and mycophenolate) have been used to treat cholestasis associated with PBC. Sulindac, bezafibrate, tamoxifen, and lamivudine have also been shown to treat or ameliorate cholestasis associated with PBC.

### Progressive Familial Intrahepatic Cholestasis (PFIC)

PFIC is a group of rare autosomal recessive disorders caused by defects in bile formation. PFIC causes progressive liver disease typically leading to liver failure. In people with PFIC, liver cells are less able to secrete bile. The resulting buildup of bile causes liver disease in affected individuals. Signs and symptoms of PFIC typically begin in infancy. Patients experience severe itching, jaundice, failure to grow at the expected rate (failure to thrive), and an increasing inability of the liver to function (liver failure). The disease is estimated to affect one in every 50,000 to 100,000 births in the United States and Europe. Six types of PFIC have been genetically identified, all of which are similarly characterized by impaired bile flow and progressive liver disease.

Children typically present in the first year of life, initially with jaundice, subsequently with other features of cholestasis; notably, pruritus and deficiency of fat-soluble vitamins (FSV). Several subtypes of PFIC are associated with significant risk of hepatocellular carcinoma. Due to failure of aggressive management in addressing symptoms, many patients undergo surgical interruption of the enterohepatic circulation of bile acids or liver transplantation. There is clearly a significant unmet need for treatments of both pruritus and the underlying liver disease.

While the prevention of liver disease progression is always paramount, early management of PFIC is largely through nutritional support and the treatment of pruritus. Medical treatment of cholestatic pruritus is particularly unsatisfactory and includes the use of rifampicin, ursodeoxycholic acid, bile acid binding resins, inhibitors of serotonin reuptake, an opioid antagonist (naloxone), and antipruritics—notably, antihistamines. The failure of medical treatment of pruritus has led to surgical interventions. Short of transplantation, the mainstay of surgical management has been depletion of the bile salt pool size through surgical biliary diversion (SBD). A large, retrospective analysis has recently shown that SBD extends transplant-free survival in patients with nt-BSEP deficiency by up to 15 years. (van Wessel D B E, Thompson R J, Gonzales E, Jankowska I, Sokal E, Grammatikopoulos T, et al. Genotype correlates with the natural history of severe bile salt export pump deficiency. J Hepatol 2020; 73:84-93) Furthermore, this study also showed that reduction of the serum bile acids (sBA), after surgery, by 75% or to less than 102 μmol/L, is associated with long-term native liver survival. Depletion of bile salts, through exteriorization of bile or through prevention of uptake in the terminal ileum, is dependent on some residual bile salt excretion into bile. The retrospective analysis of surgical management of PFIC confirmed this hypothesis and showed that patients with t-BSEP did not respond and progressed, requiring liver transplantation.

Given the limited efficacy of currently available antipruritic medications, along with the risks and burden of surgical interventions, there remains a high unmet need for alternative treatments for patients with PFIC. Pharmacological interruption of the enterohepatic bile acid recirculation has the potential to reduce the bile salt pool size, alleviate cholestatic pruritus, prevent liver damage, and reduce the need for surgical intervention. Maralixibat, a minimally absorbed, selective inhibitor of the ileal bile acid transporter (MAT), reduces sBA levels and improves growth in patients with cholestatic liver disease, as demonstrated in previous studies in children with Alagille syndrome. Maralixibat is currently FDA-approved for the treatment of cholestatic pruritus in patients with Alagille syndrome aged 1 year and older.

### PFIC 1

PFIC 1 (also known as, Byler disease or FIC1 deficiency) is associated with mutations in the ATP8B1 gene (also designated as FIC1). This gene, which encodes a P-type ATPase, is located on human chromosome 18 and is also mutated in the milder phenotype, benign recurrent intrahepatic cholestasis type 1 (BRIO) and in Greenland familial cholestasis. FIC1 protein is located on the canalicular membrane of the hepatocyte but within the liver it is mainly expressed in cholangiocytes. P-type ATPase appears to be an aminophospholipid transporter responsible for maintaining the enrichment of phosphatidylserine and phophatidylethanolamme on the inner leaflet of the plasma membrane in comparison of the outer leaflet. The asymmetric distribution of lipids in the membrane bilayer plays a protective role against high bile salt concentrations in the canalicular lumen. The abnormal protein function may indirectly disturb the biliary secretion of bile acids. The anomalous secretion of bile acids/salts leads to hepatocyte bile acid overload.

PFIC 1 typically presents in infants (e.g., age 6-18 months). The infants may show signs of pruritus, jaundice, abdominal distension, diarrhea, malnutrition, and shortened stature. Biochemically, individuals with PFIC 1 have elevated serum transaminases, elevated bilirubin, elevated serum bile acid levels, and low levels of gammaGT. The individual may also have liver fibrosis. Individuals with PFIC 1 typically do not have bile duct proliferation. Most individuals with PFIC 1 will develop end-stage liver disease by 10 years of age. No medical treatments have proven beneficial for the long-term treatment of PFIC 1. In order to reduce extrahepatic symptoms (e.g., malnutrition and failure to thrive), children are often administered medium chain triglycerides and fat-soluble vitamins. Ursodiol has not been demonstrated as effective in individuals with PFIC 1.

### PFIC 2

PFIC 2 (also known as, Byler Syndrome or BSEP deficiency) is associated with mutations in the ABCB11 gene (also designated BSEP). The ABCB11 gene encodes the ATP-dependent canalicular bile salt export pump (BSEP) of human liver and is located on human chromosome 2. BSEP protein, expressed at the hepatocyte canalicular membrane, is the major exporter of primary bile acids/salts against extreme concentration gradients. Mutations in this protein responsible for the decreased biliary bile salt secretion described in affected patients, leading to decreased bile flow and accumulation of bile salts inside the hepatocyte with ongoing severe hepatocellular damage.

PFIC 2 typically presents in infants (e.g., age 6-18 months). The infants may show signs of pruritus. Biochemically, individuals with PFIC 2 have elevated serum transaminases, elevated bilirubin, elevated serum bile acid levels, and low levels of gammaGT. The individual may also have portal inflammation and giant cell hepatitis. Further, individuals often develop hepatocellular carcinoma. No medical treatments have proven beneficial for the long-term treatment of PFIC 2. In order to reduce extrahepatic symptoms (e.g., malnutrition and failure to thrive), children are often administered medium chain triglycerides and fat-soluble vitamins. The PFIC 2 patient population accounts for approximately 60% of the PFIC population.

### PFIC 3

PFIC 3 (also known as MDR3 deficiency) is caused by a genetic defect in the ABCB4 gene (also designated MDR3) located on chromosome 7. Class III Multidrug Resistance (MDR3) P-glycoprotein (P-gp), is a phospholipid translocator involved in biliary phospholipid (phosphatidylcholine) excretion in the canlicular membrane of the hepatocyte. PFIC 3 results from the toxicity of bile in which detergent bile salts are not inactivated by phospholipids, leading to bile canaliculi and biliary epithelium injuries.

PFIC 3 also presents in early childhood. As opposed to PFIC 1 and PFIC 2, individuals have elevated gammaGT levels. Individuals also have portal inflammation, fibrosis, cirrhosis, and massive bile duct proliferation. Individuals may also develop intrahepatic gallstone disease. Ursodiol has been effective in treating or ameliorating PFIC 3.

### Benign Recurrent Intrahepatic Cholestasis (BRIC)

### BRIC 1

BRIC1 is caused by a genetic defect of the FIC1 protein in the canalicular membrane of hepatocytes. BRIC1 is typically associated with normal serum cholesterol and γ-glutamyltranspeptidase levels, but elevated serum bile salts. Residual FIC1 expression and function is associated with BRIC1. Despite recurrent attacks of cholestasis or cholestatic liver disease, there is no progression to chronic liver disease in a majority of patients. During the attacks, the patients are severely jaundiced and have pruritis, steatorrhea, and weight loss. Some patients also have renal stones, pancreatitis, and diabetes.

### BRIC 2

BRIC2 is caused by mutations in ABCB11, leading to defective BSEP expression and/or function in the canalicular membrane of hepatocytes.

### BRIC 3

BRIC3 is related to the defective expression and/or function of MDR3 in the canalicular membrane of hepatocytes. Patients with MDR3 deficiency usually display elevated serum γ-glutamyltranspeptidase levels in the presence of normal or slightly elevated bile acid levels.

### Dubin-Johnson Syndrome (DJS)

DJS is characterized by conjugated hyperbilirubinemia due to inherited dysfunction of MRP2. Hepatic function is preserved in affected patients. Several different mutations have been associated with this condition, resulting either in the complete absence of immunohistochemically detectable MRP2 in affected patients or impaired protein maturation and sorting.

### Acquired Cholestatic Disease

### Primary Biliary Cirrhosis (PBC)

PBC is a chronic inflammatory hepatic disorder slowly progressing to end stage liver failure in most of the affected patients. In PBC, the inflammatory process affects predominantly the small bile ducts.

### Primary Sclerosing Cholangitis (PSC)

PSC is a chronic inflammatory hepatic disorder slowly progressing to end stage liver failure in most of the affected patients. In PSC inflammation, fibrosis and obstruction of large and medium sized intra- and extrahepatic ductuli is predominant.

PSC is characterized by progressive cholestasis. Cholestasis can often lead to severe pruritus which significantly impairs quality of life.

### Intrahepatic Cholestasis of Pregnancy (ICP)

ICP is characterized by occurrence of transient cholestasis or cholestatic liver disease in pregnant women typically occurring in the third trimester of pregnancy, when the circulating levels of estrogens are high. ICP is associated with pruritis and biochemical cholestasis or cholestatic liver disease of varying severity and constitutes a risk factor for prematurity and intrauterine fetal death. A genetic predisposition has been suspected based upon the strong regional clustering, the higher prevalence in female family members of patients with ICP and the susceptibility of ICP patients to develop intrahepatic cholestasis or cholestatic liver disease under other hormonal challenges such as oral contraception. A heterogeneous state for an MDR3 gene defect may represent a genetic predisposition.

### Gallstone Disease

Gallstone disease is one of the most common and costly of all digestive diseases with a prevalence of up to 17% in Caucasian women. Cholesterol containing gallstones are the major form of gallstones and supersaturation of bile with cholesterol is therefore a prerequisite for gallstone formation. ABCB4 mutations may be involved in the pathogenesis of cholesterol gallstone disease.

### Drug Induced Cholestasis

Inhibition of BSEP function by drugs is an important mechanism of drug-induced cholestasis, leading to the hepatic accumulation of bile salts and subsequent liver cell damage. Several drugs have been implicated in BSEP inhibition. Most of these drugs, such as rifampicin, cyclosporine, glibenclamide, or troglitazone directly cis-inhibit ATP-dependent taurocholate transport in a competitive manner, while estrogen and progesterone metabolites indirectly trans-inhibits BSEP after secretion into the bile canaliculus by Mrp2. Alternatively, drug-mediated stimulation of MRP2 can promote cholestasis or cholestatic liver disease by changing bile composition.

### Total Parenteral Nutrition Associated Cholestasis

TPNAC is one of the most serious clinical scenarios where cholestasis or cholestatic liver disease occurs rapidly and is highly linked with early death. Infants, who are usually premature and who have had gut resections are dependent upon TPN for growth and frequently develop cholestasis or cholestatic liver disease that rapidly progresses to fibrosis, cirrhosis, and portal hypertension, usually before 6 months of life. The degree of cholestasis or cholestatic liver disease and chance of survival in these infants have been linked to the number of septic episodes, likely initiated by recurrent bacterial translocation across their gut mucosa. Although there are also cholestatic effects from the intravenous formulation in these infants, septic mediators likely contribute the most to altered hepatic function.

### Total Parenteral Nutrition Associated Cholestasis

TPNAC is one of the most serious clinical scenarios where cholestasis or cholestatic liver disease occurs rapidly and is highly linked with early death. Infants, who are usually premature and who have had gut resections are dependent upon TPN for growth and frequently develop cholestasis or cholestatic liver disease that rapidly progresses to fibrosis, cirrhosis, and portal hypertension, usually before 6 months of life. The degree of cholestasis or cholestatic liver disease and chance of survival in these infants have been linked to the number of septic episodes, likely initiated by recurrent bacterial translocation across their gut mucosa. Although there are also cholestatic effects from the intravenous formulation in these infants, septic mediators likely contribute the most to altered hepatic function.

### Alagille Syndrome (ALGS)

Alagille syndrome is a genetic disorder that affects the liver and other organs. ALGS is also known as syndromic intrahepatic bile duct paucity or arteriohepatic dysplasia. ALGS is a rare genetic disorder in which bile ducts are abnormally narrow, malformed, and reduced in number, which leads to bile accumulation in the liver and ultimately progressive liver disease. ALGS is autosomal dominant, caused by mutations in JAG1 (>90% of cases) or NOTCH2. The estimated incidence of ALGS is one in every 30,000 or 50,000 births in the United States and Europe. In patients with ALGS, multiple organ systems may be affected by the mutation, including the liver, heart, kidneys and central nervous system. The accumulation of bile acids prevents the liver from working properly to eliminate waste from the bloodstream and leads to progressive liver disease that ultimately requires liver transplantation in 15% to 47% of patients. Signs and symptoms arising from liver damage in ALGS may include jaundice, pruritus and xanthomas, and decreased growth. The pruritus experienced by patients with ALGS is among the most severe in any chronic liver disease and is present in most affected children by the third year of life.

ALGS often presents during infancy (e.g., age 6-18 months) through early childhood (e.g., age 3-5 years) and may stabilize after the age of 10. Symptoms may include chronic progressive cholestasis, ductopenia, jaundice, pruritus, xanthomas, congenital heart problems, paucity of intrahepatic bile ducts, poor linear growth, hormone resistance, posterior embryotoxon, Axenfeld anomaly, retinitis pigmentosa, pupillary abnormalities, cardiac murmur, atrial septal defect, ventricular septal defect, patent ductus arteriosus, and Tetralogy of Fallot. Individuals diagnosed with Alagille syndrome have been treated with ursodiol, hydroxyzine, cholestyramine, rifampicin, and phenobarbitol. Due to a reduced ability to absorb fat-soluble vitamins, individuals with Alagille Syndrome are further administered high dose multivitamins.

### Biliary Atresia

Biliary atresia is a life-threatening condition in infants in which the bile ducts inside or outside the liver do not have normal openings. With biliary atresia, bile becomes trapped, builds up, and damages the liver. The damage leads to scarring, loss of liver tissue, and cirrhosis. Without treatment, the liver eventually fails, and the infant needs a liver transplant to stay alive. The two types of biliary atresia are fetal and perinatal. Fetal biliary atresia appears while the baby is in the womb. Perinatal biliary atresia is much more common and does not become evident until 2 to 4 weeks after birth.

### Post-Kasai Biliary Atresia

Biliary atresia is treated with surgery called the Kasai procedure or a liver transplant. The Kasai procedure is usually the first treatment for biliary atresia. During a Kasai procedure, the pediatric surgeon removes the infant's damaged bile ducts and brings up a loop of intestine to replace them. While the Kasai procedure can restore bile flow and correct many problems caused by biliary atresia, the surgery doesn't cure biliary atresia. If the Kasai procedure is not successful, infants usually need a liver transplant within 1 to 2 years. Even after a successful surgery, most infants with biliary atresia slowly develop cirrhosis over the years and require a liver transplant by adulthood. Possible complications after the Kasai procedure include ascites, bacterial cholangitis, portal hypertension, and pruritis.

### Post Liver Transplantation Biliary Atresia

If the atresia is complete, liver transplantation is the only option. Although liver transplantation is generally successful at treating biliary atresia, liver transplantation may have complications such as organ rejection. Also, a donor liver may not become available. Further, in some patients, liver transplantation may not be successful at curing biliary atresia.

### Xanthoma

Xanthoma is a skin condition associated with cholestatic liver diseases, in which certain fats build up under the surface of the skin. Cholestasis results in several disturbances of lipid metabolism resulting in formation of an abnormal lipid particle in the blood called lipoprotein X. Lipoprotein X is formed by regurgitation of bile lipids into the blood from the liver and does not bind to the LDL receptor to deliver cholesterol to cells throughout the body as does normal LDL. Lipoprotein X increases liver cholesterol production by fivefold and blocks normal removal of lipoprotein particles from the blood by the liver.

### General Definitions

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “baseline” or “pre-administration baseline,” as used herein, refers to information gathered at the beginning of a study or an initial known value which is used for comparison with later data. A baseline is an initial measurement of a measurable condition that is taken at an early time point and used for comparison over time to look for changes in the measurable condition. For example, serum bile acid concentration in a patient before administration of a drug (baseline) and after administration of the drug. Baseline is an observation or value that represents the normal or beginning level of a measurable quality, used for comparison with values representing response to intervention or an environmental stimulus. The baseline is time “zero”, before participants in a study receive an experimental agent or intervention, or negative control. For example, “baseline” may refer in some instances 1) to the state of a measurable quantity just prior to the initiation of a clinical study or 2) the state of a measurable quantity just prior to altering a dosage level or composition administered to a patient from a first dosage level or composition to a second dosage level or composition.

The terms “level” and “concentration,” as used herein, are used interchangeably. For example, “high serum levels of bilirubin” may alternatively be phrased “high serum concentrations of bilirubin.”

The terms “normalized” or “normal range,” as used herein, indicates age-specific values that are within a range corresponding to a healthy individual (i.e., normal or normalized values). For example, the phrase “serum bilirubin concentrations were normalized within three weeks” means that serum bilirubin concentrations fell within a range known in the art to correspond to that of a healthy individual (i.e., within a normal and not e.g. an elevated range) within three weeks. In various embodiments, a normalized serum bilirubin concentration is from about 0.1 mg/dL to about 1.2 mg/dL. In various embodiments, a normalized serum bile acid concentration is from about 0 μmol/L to about 25 μmol/L.

The terms “ITCHRO(OBS)” and “ITCHRO” (alternatively, “ItchRO(Pt)”) as used herein, are used interchangeably with the qualification that the ITCHRO(OBS) scale is used to measure severity of pruritus in children under the age of 18 and the ITCHRO scale is used to measure severity of pruritus in adults of at least 18 years of age. Therefore, where ITCHRO(OBS) scale is mentioned with regard to an adult patient, the ITCHRO scale is the scale being indicated. Similarly, whenever the ITCHRO scale is mentioned with regard to a pediatric patient, the ITCHRO(OBS) scale is usually the scale being indicated (some older children were permitted to report their own scores as ITCHRO scores. The ITCHRO(OBS) scale ranges from 0 to 4 and the ITCHRO scale ranges from 0 to 10.

The term “bile acid” or “bile acids,” as used herein, includes steroid acids (and/or the carboxylate anion thereof), and salts thereof, found in the bile of an animal (e.g., a human), including, by way of non-limiting example, cholic acid, cholate, deoxycholic acid, deoxycholate, hyodeoxycholic acid, hyodeoxycholate, glycocholic acid, glycocholate, taurocholic acid, taurocholate, chenodeoxycholic acid, ursodeoxycholic acid, ursodiol, a tauroursodeoxycholic acid, a glycoursodeoxycholic acid, a 7-B-methyl cholic acid, a methyl lithocholic acid, chenodeoxycholate, lithocholic acid, lithocolate, and the like. Taurocholic acid and/or taurocholate are referred to herein as TCA. Any reference to a bile acid used herein includes reference to a bile acid, one and only one bile acid, one or more bile acids, or to at least one bile acid. Therefore, the terms “bile acid,” “bile salt,” “bile acid/salt,” “bile acids,” “bile salts,” and “bile acids/salts” are, unless otherwise indicated, utilized interchangeably herein. Any reference to a bile acid used herein includes reference to a bile acid or a salt thereof. Furthermore, pharmaceutically acceptable bile acid esters are optionally utilized as the “bile acids” described herein, e.g., bile acids/salts conjugated to an amino acid (e.g., glycine or taurine). Other bile acid esters include, e.g., substituted or unsubstituted alkyl ester, substituted or unsubstituted heteroalkyl esters, substituted or unsubstituted aryl esters, substituted or unsubstituted heteroaryl esters, or the like. For example, the term “bile acid” includes cholic acid conjugated with either glycine or taurine: glycocholate and taurocholate, respectively (and salts thereof). Any reference to a bile acid used herein includes reference to an identical compound naturally or synthetically prepared. Furthermore, it is to be understood that any singular reference to a component (bile acid or otherwise) used herein includes reference to one and only one, one or more, or at least one of such components. Similarly, any plural reference to a component used herein includes reference to one and only one, one or more, or at least one of such components, unless otherwise noted.

The term “subject”, “patient”, “participant”, or “individual” are used interchangeably herein and refer to mammals and non-mammals, e.g., suffering from a disorder described herein. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The term “about,” as used herein, includes any value that is within 10% of the described value.

The term “composition,” as used herein includes the disclosure of both a composition and a composition administered in a method as described herein. Furthermore, in some embodiments, the composition of the present invention is or comprises a “formulation,” an oral dosage form or a rectal dosage form as described herein.

The terms “treat,” “treating” or “treatment,” and other grammatical equivalents as used herein, include alleviating, inhibiting or reducing symptoms, reducing or inhibiting severity of, reducing incidence of, reducing or inhibiting recurrence of, delaying onset of, delaying recurrence of, abating or ameliorating a disease or condition symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

The terms “effective amount” or “therapeutically effective amount” as used herein, refer to a sufficient amount of at least one agent (e.g., a therapeutically active agent) being administered which achieve a desired result in a subject or individual, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study. In some embodiments, a “therapeutically effective amount,” or an “effective amount” of an IBAT inhibitor refers to a sufficient amount of an IBAT inhibitor to treat cholestasis or a cholestatic liver disease in a subject or individual.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein are found in sources e.g., Goodman and Gilman, The Pharmacological Basis of Therapeutics, current ed.; Pergamon; and Remington's, Pharmaceutical Sciences (current edition), Mack Publishing Co., Easton, Pa., all of which are incorporated herein by reference in their entirety for all purposes. In certain embodiments, the agents and compositions described herein are administered orally.

The term “IBAT inhibitor” refers to a compound that inhibits ileal bile acid transport or any recuperative bile salt transport. The term “ASBT inhibitor” refers to a compound that inhibits ileal bile transport or any recuperative bile salt transport. The term Apical Sodium-dependent Bile Transporter (ASBT) is used interchangeably with the term Ileal Bile Acid Transporter (IBAT).

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, subsalicylate, maleate, laurate, malate, fumarate, succinate, tartrate, amsonate, pamoate, p-tolunenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like.

As used herein the term “fasted state” is defined as a state one in which a subject has completely digested and absorbed the last meal, and the subject's insulin levels are at a low or baseline level. In some embodiments, a fasted state is defined as a state of not consuming any food for at least 4 hours for a subject 18 years or over. In some embodiments, a fasted state is defined as a state of not consuming any food for at least 2 hours for a pediatric subject. In some embodiments, a fasted state is defined as a state of about 30 minutes before a meal.

As used herein, a fasted patient is defined as a patient who has not eaten any food, i.e., has fasted for at least 4 hours before the administration of the IBAT inhibitor (for a subject 18 years or over) or at least 2 hours before the administration of the IBAT inhibitor (for a pediatric subject), and at least 30 minutes after the administration of the IBAT inhibitor. The IBAT inhibitor is optionally administered with water during the fasting period, and water can be allowed ad libitum.

### Bile Acid

Bile contains water, electrolytes and a numerous organic molecules including bile acids, cholesterol, phospholipids and bilirubin. Bile is secreted from the liver and stored in the gall bladder, and upon gall bladder contraction, due to ingestion of a fatty meal, bile passes through the bile duct into the intestine. Bile acids/salts are critical for digestion and absorption of fats and fat-soluble vitamins in the small intestine. Adult humans produce 400 to 800 mL of bile daily. The secretion of bile can be considered to occur in two stages. Initially, hepatocytes secrete bile into canaliculi, from which it flows into bile ducts and this hepatic bile contains large quantities of bile acids, cholesterol and other organic molecules. Then, as bile flows through the bile ducts, it is modified by addition of a watery, bicarbonate-rich secretion from ductal epithelial cells. Bile is concentrated, typically five-fold, during storage in the gall bladder.

The flow of bile is lowest during fasting, and a majority of that is diverted into the gallbladder for concentration. When chyme from an ingested meal enters the small intestine, acid and partially digested fats and proteins stimulate secretion of cholecystokinin and secretin, both of which are important for secretion and flow of bile. Cholecystokinin (cholecysto=gallbladder and kinin=movement) is a hormone which stimulates contractions of the gallbladder and common bile duct, resulting in delivery of bile into the gut. The most potent stimulus for release of cholecystokinin is the presence of fat in the duodenum. Secretin is a hormone secreted in response to acid in the duodenum, and it simulates biliary duct cells to secrete bicarbonate and water, which expands the volume of bile and increases its flow out into the intestine.

Bile acids/salts are derivatives of cholesterol. Cholesterol, ingested as part of the diet or derived from hepatic synthesis, are converted into bile acids/salts in the hepatocyte. Examples of such bile acids/salts include cholic and chenodeoxycholic acids, which are then conjugated to an amino acid (such as glycine or taurine) to yield the conjugated form that is actively secreted into cannaliculi. The most abundant of the bile salts in humans are cholate and deoxycholate, and they are normally conjugated with either glycine or taurine to give glycocholate or taurocholate respectively.

Free cholesterol is virtually insoluble in aqueous solutions, however in bile it is made soluble by the presence of bile acids/salts and lipids. Hepatic synthesis of bile acids/salts accounts for the majority of cholesterol breakdown in the body. In humans, roughly 500 mg of cholesterol are converted to bile acids/salts and eliminated in bile every day. Therefore, secretion into bile is a major route for elimination of cholesterol. Large amounts of bile acids/salts are secreted into the intestine every day, but only relatively small quantities are lost from the body. This is because approximately 95% of the bile acids/salts delivered to the duodenum are absorbed back into blood within the ileum, by a process is known as “Enterohepatic Recirculation”.

Venous blood from the ileum goes straight into the portal vein, and hence through the sinusoids of the liver. Hepatocytes extract bile acids/salts very efficiently from sinusoidal blood, and little escapes the healthy liver into systemic circulation. Bile acids/salts are then transported across the hepatocytes to be resecreted into canaliculi. The net effect of this enterohepatic recirculation is that each bile salt molecule is reused about 20 times, often two or three times during a single digestive phase. Bile biosynthesis represents the major metabolic fate of cholesterol, accounting for more than half of the approximate 800 mg/day of cholesterol that an average adult uses up in metabolic processes. In comparison, steroid hormone biosynthesis consumes only about 50 mg of cholesterol per day. Much more that 400 mg of bile salts is required and secreted into the intestine per day, and this is achieved by re-cycling the bile salts. Most of the bile salts secreted into the upper region of the small intestine are absorbed along with the dietary lipids that they emulsified at the lower end of the small intestine. They are separated from the dietary lipid and returned to the liver for re-use. Recycling thus enables 20-30 g of bile salts to be secreted into the small intestine each day.

Bile acids/salts are amphipathic, with the cholesterol-derived portion containing both hydrophobic (lipid soluble) and polar (hydrophilic) moieties while the amino acid conjugate is generally polar and hydrophilic. This amphipathic nature enables bile acids/salts to carry out two important functions: emulsification of lipid aggregates and solubilization and transport of lipids in an aqueous environment. Bile acids/salts have detergent action on particles of dietary fat which causes fat globules to break down or to be emulsified. Emulsification is important since it greatly increases the surface area of fat available for digestion by lipases which cannot access the inside of lipid droplets. Furthermore, bile acids/salts are lipid carriers and are able to solubilize many lipids by forming micelles and are critical for transport and absorption of the fat-soluble vitamins.

The term “non-systemic” or “minimally absorbed,” as used herein, refers to low systemic bioavailability and/or absorption of an administered compound. In some embodiments a non-systemic compound is a compound that is substantially not absorbed systemically. In some embodiments, IBAT inhibitor compositions described herein deliver the IBAT inhibitor to the distal ileum, colon, and/or rectum and not systemically (e.g., a substantial portion of the IBAT inhibitor is not systemically absorbed. In some embodiments, the systemic absorption of a non-systemic compound is <0.1%, <0.3%, <0.5%, <0.6%, <0.7%, <0.8%, <0.9%, <1%, <1.5%, <2%, <3%, or <5% of the administered dose (wt. % or mol %). In some embodiments, the systemic absorption of a non-systemic compound is <10% of the administered dose. In some embodiments, the systemic absorption of a non-systemic compound is <15% of the administered dose. In some embodiments, the systemic absorption of a non-systemic compound is <25% of the administered dose. In some embodiments, the systemic absorption of a non-systemic compound is <30% of the administered dose. In an alternative approach, a non-systemic IBAT inhibitor is a compound that has lower systemic bioavailability relative to the systemic bioavailability of a systemic IBAT inhibitor (e.g., compound 100A, 100C). In some embodiments, the bioavailability of a non-systemic IBAT inhibitor described herein is <30%, <40%, <50%, <60%, or <70% of the bioavailability of a systemic IBAT inhibitor (e.g., maralixibat or volixibat).

In another alternative approach, compositions described herein are formulated to deliver <10% of the administered dose of the IBAT inhibitor systemically. In some embodiments, the compositions described herein are formulated to deliver <20% of the administered dose of the IBAT inhibitor systemically. In some embodiments, the compositions described herein are formulated to deliver <30% of the administered dose of the IBAT inhibitor systemically. In some embodiments, the compositions described herein are formulated to deliver <40% of the administered dose of the IBAT inhibitor systemically. In some embodiments, the compositions described herein are formulated to deliver <50% of the administered dose of the IBAT inhibitor systemically. In some embodiments, the compositions described herein are formulated to deliver <60% of the administered dose of the IBAT inhibitor systemically. In some embodiments, the compositions described herein are formulated to deliver <70% of the administered dose of the IBAT inhibitor systemically. In some embodiments, systemic absorption is determined in any suitable manner, including the total circulating amount, the amount cleared after administration, or the like.

### Event-Free Survival (EFS)

In various embodiments of methods of the present invention, administration of IBAT inhibitors to a subject increases event-free survival (EFS). In certain embodiments, administration of the IBAT inhibitor increases event-free survival (EFS) of the subject by reducing one or more of:

a) total bilirubin (TB); b) total serum bile acids (sBA), and c) pruritus score as measured by an Itch Reported Outcome (ItchRO) severity assessment tool.

In certain embodiments, EFS comprises survival in the absence of one or more of hepatic decompensation, surgical biliary diversion, liver transplantation or death. In certain embodiments, hepatic decompensation comprises variceal bleeding and/or ascites requiring therapy.

In certain embodiments, the present disclosure provides methods for providing a prediction of response to an IBAT inhibitor therapy for treatment of cholestatic liver disease in a subject in need thereof by predicting event-free survival (EFS), the method comprising: obtaining one or more of total bilirubin (TB) data, total serum bile acids (sBA) data, pruritus reduction data and age of the subject at initiation of treatment with the IBAT inhibitor, and using the data obtained for the subject to predict EFS.

In certain embodiments, the EFS is predicted when the TB is less than about 6.5 mg/dL. In certain embodiments, the EFS is predicted when the TB is about 6 mg/dL. In certain embodiments, the EFS is predicted when the TB is less than about 5 mg/dL. In certain embodiments, the EFS is predicted when the TB is less than about 4 mg/dL. In certain embodiments, the EFS is predicted when the TB is less than about 3 mg/dL. In certain embodiments, the EFS is predicted when the TB is less than about 2 mg/dL. In certain embodiments, the EFS is predicted when the TB is less than about 1 mg/ml. In certain embodiments, the EFS is predicted when the TB is less than about 0.1 mg/ml.

In certain embodiments, the EFS is predicted when the sBA level after treatment with the IBAT inhibitor is less than about 200 μmol/L. In certain embodiments, the EFS is predicted when the sBA level after treatment with the IBAT inhibitor is less than about 150 μmol/L. In certain embodiments, the EFS is predicted when the sBA level after treatment with the IBAT inhibitor is less than about 100 μmol/L. In certain embodiments, the EFS is predicted when the sBA level after treatment with the IBAT inhibitor is less than about 50 μmol/L. In certain embodiments, the EFS is predicted when the sBA level after treatment with the IBAT inhibitor is less than about 20 μmol/L. In certain embodiments, the EFS is predicted when the sBA level after treatment with the IBAT inhibitor is less than about 10 μmol/L. In certain embodiments, the EFS is predicted when the sBA level after treatment with the IBAT inhibitor is less than about 5 μmol/L.

In certain embodiments, the sBA level is determined 18 weeks after initiation of the IBAT inhibitor treatment. In certain embodiments, the sBA level is determined 24 weeks after initiation of the IBAT inhibitor treatment. In certain embodiments, the sBA level is determined 48 weeks after initiation of the IBAT inhibitor treatment. In certain embodiments, the sBA level is determined at about 100 weeks after initiation of the IBAT inhibitor treatment. In certain embodiments, the sBA level is determined at about 150 weeks after initiation of the IBAT inhibitor treatment. In certain embodiments, the sBA level is determined at about 200 weeks after initiation of the IBAT inhibitor treatment. In certain embodiments, the sBA level is determined at about 250 weeks after initiation of the IBAT inhibitor treatment. In certain embodiments, the sBA level is determined at about 300 weeks after initiation of the IBAT inhibitor treatment. In certain embodiments, the sBA level is determined at about 300 weeks after initiation of the IBAT inhibitor treatment. In certain embodiments, the sBA level is determined at about 350 weeks after initiation of the IBAT inhibitor treatment. In certain embodiments, the sBA level is determined at about 400 weeks after initiation of the IBAT inhibitor treatment. In certain embodiments, the sBA level is determined at about 450 weeks after initiation of the IBAT inhibitor treatment. In certain embodiments, the sBA level is determined at about 500 weeks after initiation of the IBAT inhibitor treatment. In certain embodiments, the sBA level is determined from about 18 weeks to about 500 weeks after initiation of the IBAT inhibitor treatment. In certain embodiments, the sBA level is determined from about 48 weeks to about 500 weeks after initiation of the IBAT inhibitor treatment.

In certain embodiments, the EFS is predicted when the pruritus reduction is more than about 1 point after treatment with the IBAT inhibitor compared to the pruritus at the time of first administration of the IBAT inhibitor, wherein the pruritus is measured by an Itch Reported Outcome (ItchRO) severity assessment tool.

In certain embodiments, the pruritus is determined 18 weeks after the initiation of the IBAT inhibitor treatment. In certain embodiments, the pruritus is determined 24 weeks after the initiation of the IBAT inhibitor treatment. In certain embodiments, the pruritus is determined 48 weeks after the initiation of the IBAT inhibitor treatment. In certain embodiments, the pruritus is determined at about 100 weeks after initiation of the IBAT inhibitor treatment. In certain embodiments, the pruritus is determined at about 150 weeks after initiation of the IBAT inhibitor treatment. In certain embodiments, the pruritus is determined at about 200 weeks after initiation of the IBAT inhibitor treatment. In certain embodiments, the pruritus is determined at about 250 weeks after initiation of the IBAT inhibitor treatment. In certain embodiments, the pruritus is determined at about 300 weeks after initiation of the IBAT inhibitor treatment. In certain embodiments, the pruritus is determined at about 300 weeks after initiation of the IBAT inhibitor treatment. In certain embodiments, the pruritus is determined at about 350 weeks after initiation of the IBAT inhibitor treatment. In certain embodiments, the pruritus is determined at about 400 weeks after initiation of the IBAT inhibitor treatment. In certain embodiments, the pruritus is determined at about 450 weeks after initiation of the IBAT inhibitor treatment. In certain embodiments, the pruritus is determined at about 500 weeks after initiation of the IBAT inhibitor treatment. In certain embodiments, the pruritus is determined from about 18 weeks to about 500 weeks after initiation of the IBAT inhibitor treatment. In certain embodiments, the pruritus is determined from about 48 weeks to about 500 weeks after initiation of the IBAT inhibitor treatment.

In certain embodiments, the EFS is predicted when the age of the subject at the time of initiation of treatment is equal to or higher than about 36 months.

### Health-Related Quality of Life (HRQoL)

In various embodiments of methods of the present invention, administration of IBAT inhibitors to a subject results in improved health-related quality of life (HRQoL).

In certain embodiments, the HRQoL is determined by using the Itch Reported Outcome (ItchRO), Pediatric Quality of Life Inventory Generic Core (PedsQL), Family Impact (FI), and Multidimensional Fatigue (MF) scale scores.

In certain embodiments, the ItchRO scale score ranges from 0, wherein 0=none, to 4, wherein 4=very severe. In certain embodiments, clinically meaningful pruritus response is defined as a ≥1 point reduction in the ItchRO, from baseline to week 48 of treatment.

In certain embodiments, the PedsQL scale score ranges from 0-100, wherein 100=best quality of life.

### IBAT Inhibitors

In various embodiments of methods of the present invention, IBAT inhibitors are administered to a subject. IBAT inhibitors (ASBT inhibitors) reduce or inhibit bile acid recycling in the distal gastrointestinal (GI) tract, including the distal ileum, the colon and/or the rectum. Inhibition of the ileal bile acid transport interrupts the enterohepatic circulation of bile acids and results in more bile acids being excreted in the feces, leading to lower levels of bile acids systemically, thereby reducing bile acid mediated liver damage and related effects and complications. In certain embodiments, the IBAT inhibitors are systemically absorbed. In certain embodiments, the IBAT inhibitors are not systemically absorbed. In some embodiments, IBAT inhibitors described herein are modified or substituted to be non-systemic.

In certain embodiments, compounds described herein have one or more chiral centers. As such, all stereoisomers are envisioned herein. In various embodiments, compounds described herein are present in optically active or racemic forms. It is to be understood that the compounds of the present invention encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer is utilized as the therapeutic compound described herein. In certain embodiments, compounds described herein contains one or more chiral centers. These compounds are prepared by any means, including enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

In some embodiments, the IBAT inhibitor is

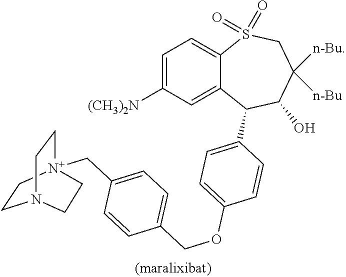

In some embodiments, the IBAT inhibitor is

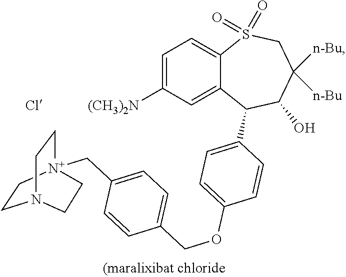

LUM-001, SHP625, lopixibat chloride), or an alternative pharmaceutically acceptable salt thereof.

In some embodiments, the IBAT inhibitor is

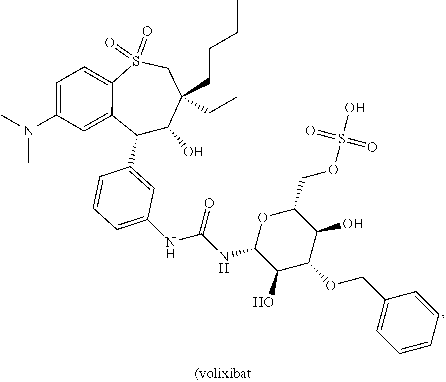 [SMILES: CCCC[C@]1(CC)CS(=O)(=O)c2ccc(N(C)C)cc2[C@@H](c2cccc(NC(=O)N[C@@H]3O[C@H](COS(=O)(=O)O)[C@@H](O)[C@H](OCc4ccccc4)[C@H]3O)c2)[C@H]1O]

(2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl) hydrogen sulfate), or a pharmaceutically acceptable salt thereof.

In some embodiments, the IBAT inhibitor is

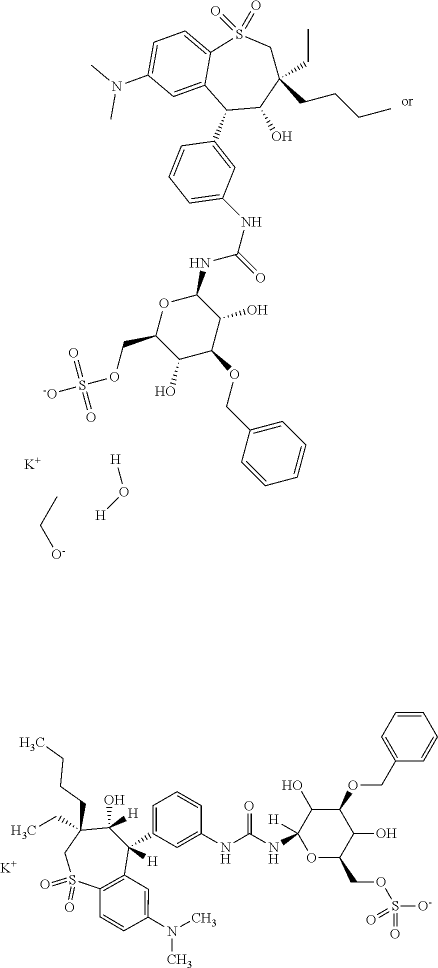

(LUM-002; SHP626; SAR548304; volixibat potassium), or an alternative pharmaceutically acceptable salt thereof.

In various embodiments the IBAT inhibitor is

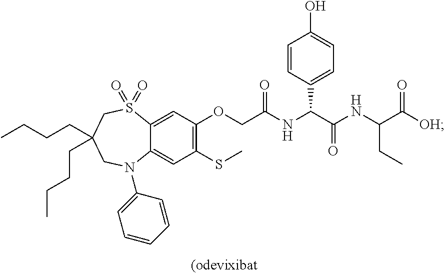 [SMILES: CCCCC1(CCCC)CN(c2ccccc2)c2cc(SC)c(OCC(=O)N[C@@H](C(=O)NC(CC)C(=O)O)c3ccc(O)cc3)cc2S(=O)(=O)C1]

AZD8294; WHO10706; AR-H064974; SCHEMBL946468; A4250; 1,1-dioxo-3,3-dibutyl-5-phenyl-7-methylthio-8-(N—{(R)-a-[N—((S)-1-carboxypropyl) carbamoyl]-4-hydroxybenzyl}carbamoylmethoxy)-2,3,4,5-tetrahydro-1,2,5-benzothiadiazepine), or a pharmaceutically acceptable salt thereof.

In some embodiments, the IBAT inhibitor is

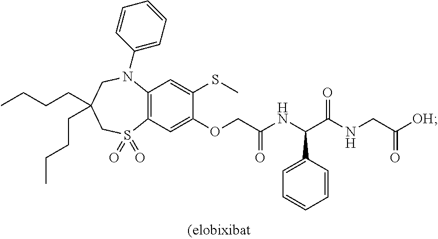 [SMILES: CCCCC1(CCCC)CN(c2ccccc2)c2cc(SC)c(OCC(=O)N[C@@H](C(=O)NCC(=O)O)c3ccccc3)cc2S(=O)(=O)C1]

2-[[(2R)-2-[[2-[(3,3-dibutyl-7-methylsulfanyl-1,1-dioxo-5-phenyl-2,4-dihydro-1λ6,5-benzothiazepin-8-yl)oxy]acetyl]amino]-2-phenylacetyl]amino]acetic acid), or a pharmaceutically acceptable salt thereof.

In some embodiments, the IBAT inhibitor is

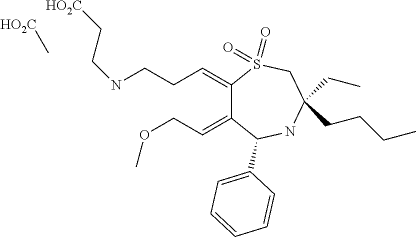

(GSK2330672; linerixibat; 3-((((3R,5R)-3-butyl-3-ethyl-7-(methyloxy)-1,1-dioxido-5-phenyl-2,3,4,5-tetrahydro-1,4-benzothiazepin-8-yl)methyl)amino)pentanedioic acid), or a pharmaceutically acceptable salt thereof.

In some embodiments, the IBAT inhibitor used in the methods or compositions of the present invention is maralixibat (e.g., as maralixibat chloride), volixibat (e.g., as volixibat potassium), or odevixibat (A4250), or a pharmaceutically acceptable salt thereof.

In some embodiments, the IBAT inhibitor used in the methods or compositions of the present invention is maralixibat, or a pharmaceutically acceptable salt thereof.

In some embodiments, the IBAT inhibitor used in the methods or compositions of the present invention is volixibat, or a pharmaceutically acceptable salt thereof.

In some embodiments, the IBAT inhibitor used in the methods or compositions of the present invention is odevixibat, or a pharmaceutically acceptable salt thereof.

In some embodiments, the IBAT inhibitor used in the methods or compositions of the present invention is elobixibat, or a pharmaceutically acceptable salt thereof.

In some embodiments, the IBAT inhibitor used in the methods or compositions of the present invention is GSK2330672, or a pharmaceutically acceptable salt thereof.

In some embodiments, the IBAT inhibitor may comprise a mixture of different IBAT inhibitors; for example, the IBAT inhibitor may be a composition comprising maralixibat, volixibat, odevixibat, GSK2330672, elobixibat, or various combinations thereof.

### Methods for Treating Cholestasis and Minimizing Gastrointestinal Adverse Effects

Provided herein is a method for treating cholestasis in a subject having a liver disease wherein the treatment increases event-free survival (EFS) of the subject. The method includes administering to a subject in need of treatment an Apical Sodium-dependent Bile Acid Transporter Inhibitor (IBAT inhibitor). The IBAT inhibitor is maralixibat or volixibat, or a pharmaceutically acceptable salt thereof. The IBAT inhibitor is administered in an amount of from about 100 μg/kg/day to about 1400 μg/kg/day.

Provided herein is a method of treating cholestatic liver disease in a subject in need thereof, the method comprising administering to the subject a therapeutically effective amount of an ileal bile acid transporter inhibitor (IBAT inhibitor), wherein the treatment results in one or more of 1) increased event-free survival (EFS) and 2) improved health-related quality of life (HRQoL).

Provided herein is a method of treating cholestatic liver disease in a subject in need thereof, the method comprising administering to the subject a therapeutically effective amount of an ileal bile acid transporter (IBAT) inhibitor, wherein the treatment increases event-free survival (EFS) of the subject by reducing one or more of:

a) total bilirubin (TB);
b) total serum bile acids (sBA), and
c) pruritus score as measured by an Itch Reported Outcome (ItchRO) severity assessment tool.

Provided herein is a method of treating Alagille syndrome in a pediatric subject in need thereof, the method comprising administering to the subject a therapeutically effective amount of maralixibat or a pharmaceutically acceptable salt thereof, wherein the increases event-free survival (EFS) of the subject for at least 18 months following the first dose of the IBAT inhibitor.

Provided herein is a method for providing a prediction of response to an IBAT inhibitor therapy for treatment of cholestatic liver disease in a subject in need thereof by predicting event-free survival (EFS), the method comprising obtaining one or more of total bilirubin (TB) data, total serum bile acids (sBA) data, pruritus reduction data and age of the subject at initiation of treatment with the IBAT inhibitor, and using the data obtained for the subject to predict EFS.

Provided herein is a method for providing a prediction of response to maralixibat therapy for treatment of Alagille syndrome in a subject in need thereof by predicting event-free survival (EFS) for 6 years following the first dose of the maralixibat, the method comprising: obtaining total bilirubin (TB) data, total serum bile acids (sBA) data, pruritus reduction data and age of the subject at initiation of treatment with the IBAT inhibitor, and using the data obtained for the subject to predict the EFS.

Provided herein is a method for treating cholestatic liver disease in a subject in need thereof, the method comprising administering a therapeutically effective amount of an IBAT inhibitor to the subject before ingestion of food, wherein the subject experiences a reduction in frequency and/or severity of one or more side effects associated with the administration of the IBAT inhibitor, and wherein the treatment increases event-free survival (EFS) of the subject. The method includes administering to a subject in need of treatment an IBAT inhibitor before ingestion of food. In certain embodiments, the IBAT inhibitor is maralixibat or volixibat, or a pharmaceutically acceptable salt thereof. The IBAT inhibitor is administered in an amount of from about 100 μg/kg/day to about 1400 μg/kg/day.

In certain embodiments, the IBAT inhibitor is administered to the subject in a fasted state. In certain embodiments, the IBAT inhibitor is administered less than about 1 minute, less than about 5 minutes, less than about 10 minutes, less than about 15 minutes, less than about 20 minutes, less than about 30 minutes or less than about 60 minutes before ingestion of food. In certain embodiments, the IBAT inhibitor is administered immediately prior to the ingestion of food.

In various embodiments, the liver disease is a cholestatic liver disease. In some embodiments, the liver disease is PFIC, ALGS, PSC, biliary atresia, intrahepatic cholestasis of pregnancy, PBC, any of the cholestatic liver diseases discussed above, or various combinations thereof.

In certain embodiments, the cholestatic liver disease is progressive familial intrahepatic cholestasis (PFIC), PFIC type 1, PFIC type 2, PFIC type 3, Alagille syndrome, Dubin-Johnson Syndrome, biliary atresia, post-Kasai biliary atresia, post-liver transplantation biliary atresia, post-liver transplantation cholestasis, post-liver transplantation associated liver disease, intestinal failure associated liver disease, bile acid mediated liver injury, pediatric primary sclerosing cholangitis, MRP2 deficiency syndrome, neonatal sclerosing cholangitis, a pediatric obstructive cholestasis, a pediatric non-obstructive cholestasis, a pediatric extrahepatic cholestasis, a pediatric intrahepatic cholestasis, a pediatric primary intrahepatic cholestasis, a pediatric secondary intrahepatic cholestasis, benign recurrent intrahepatic cholestasis (BRIC), BRIP type 1, BRIC type 2, BRIC type 3, total parenteral nutrition associated cholestasis, paraneoplastic cholestasis, Stauffer syndrome, drug-associated cholestasis, infection-associated cholestasis, or gallstone disease. In some embodiments, the cholestatic liver disease is a pediatric form of liver disease. In some embodiments, the subject has intrahepatic cholestasis of pregnancy (ICP).

In certain embodiments, a cholestatic liver disease is characterized by one or more symptoms selected from jaundice, pruritis, cirrhosis, hypercholemia, neonatal respiratory distress syndrome, lung pneumonia, increased serum concentration of bile acids, increased hepatic concentration of bile acids, increased serum concentration of bilirubin, hepatocellular injury, liver scarring, liver failure, hepatomegaly, xanthomas, malabsorption, splenomegaly, diarrhea, pancreatitis, hepatocellular necrosis, giant cell formation, hepatocellular carcinoma, gastrointestinal bleeding, portal hypertension, hearing loss, fatigue, loss of appetite, anorexia, peculiar smell, dark urine, light stools, steatorrhea, failure to thrive, and/or renal failure.

In various embodiments the liver disease is PFIC 2 and the subject has a non-truncating mutation in the ABCB11 gene. In various embodiments the non-truncating mutation in the ABCB11 gene is a missense mutation. In various embodiments the missense mutation may be selected from one of those mutations listed in Byrne, et al., “Missense Mutations and Single Nucleotide Polymorphisms in ABCB11 Impair Bile Salt Export Pump Processing and Function or Disrupt Pre-Messanger RNA Splicing,” Hepatology, 49:553-567 (2009), which is incorporated herein by reference in its entirety for all purposes.

In various embodiments the subject has a condition associated with, caused by or caused in part by a BSEP deficiency. In certain embodiments, the condition associated with, caused by or caused in part by the BSEP deficiency is neonatal hepatitis, primary biliary cirrhosis (PBC), primary sclerosing cholangitis (PSC), PFIC 2, benign recurrent intrahepatic cholestasis (BRIC), intrahepatic cholestasis of pregnancy (ICP), drug-induced cholestasis, oral-contraceptive-induced cholestasis, biliary atresia, or a combination thereof.

In various embodiments, the patient is a pediatric patient under the age of 0, 1, 2, 3, 4, 5, 6, 7, 8, 9, 10, 11, 12, 13, 14, 15, 16, 17, or 18 years old. In certain embodiments, the pediatric subject is a newborn, a pre-term newborn, an infant, a toddler, a preschooler, a school-age child, a pre-pubescent child, post-pubescent child, an adolescent, or a teenager under the age of eighteen. In some embodiments, the pediatric subject is a newborn, a pre-term newborn, an infant, a toddler, a preschooler, or a school-age child. In some embodiments, the pediatric subject is a newborn, a pre-term newborn, an infant, a toddler, or a preschooler. In some embodiments, the pediatric subject is a newborn, a pre-term newborn, an infant, or a toddler. In some embodiments, the pediatric subject is a newborn, a pre-term newborn, or an infant. In some embodiments, the pediatric subject is a newborn. In some embodiments, the pediatric subject is an infant. In some embodiments, the pediatric subject is a toddler. In various embodiments, the pediatric patient has PFIC 2, PFIC 1, or ALGS. In some embodiments, the patient is an adult over the age of 18, 20, 30, 40, 50, 60, or 70. In some patients, the adult patient has PSC. In some patients, the adult patient has PBC. In some patients, the adult patient has ICP. In some embodiments, the pediatric patient has any pediatric cholestatic condition resulting in below normal growth, height, or weight.

In certain embodiments, methods of the present invention comprise non-systemic administration of a therapeutically effective amount of an IBAT inhibitor. In certain embodiments, the methods comprise contacting the gastrointestinal tract, including the distal ileum and/or the colon and/or the rectum, of an individual in need thereof with an IBAT inhibitor. In various embodiments, the methods of the present invention cause a reduction in intraenterocyte bile acids, or a reduction in damage to hepatocellular or intestinal architecture caused by cholestasis or a cholestatic liver disease.

In various embodiments, methods of the present invention comprise delivering to ileum or colon of the individual a therapeutically effective amount of any IBAT inhibitor described herein.

In various embodiments, methods of the present invention comprise reducing damage to hepatocellular or intestinal architecture or cells from cholestasis or a cholestatic liver disease comprising administration of a therapeutically effective amount of an IBAT inhibitor. In certain embodiments, the methods of the present invention comprise reducing intraenterocyte bile acids/salts through administration of a therapeutically effective amount of an IBAT inhibitor to an individual in need thereof.

In some embodiments, methods of the present invention provide for inhibition of bile salt recycling upon administration of any of the compounds described herein to an individual. In some embodiments, an IBAT inhibitor described herein is systemically absorbed upon administration. In some embodiments, an IBAT inhibitor described herein is not absorbed systemically. In some embodiments, an IBAT inhibitor herein is administered to the individual orally. In some embodiments, an IBAT inhibitor described herein is delivered and/or released in the distal ileum of an individual.

In various embodiments, contacting the distal ileum of an individual with an IBAT inhibitor (e.g., any IBAT inhibitor described herein) inhibits bile acid reuptake and increases the concentration of bile acids/salts in the vicinity of L-cells in the distal ileum and/or colon and/or rectum, thereby reducing intraenterocyte bile acids, reducing serum and/or hepatic bile acid levels, reducing overall serum bile acid load, and/or reducing damage to ileal architecture caused by cholestasis or a cholestatic liver disease. Without being limited to any particular theory, reducing serum and/or hepatic bile acid levels ameliorates hypercholemia and/or cholestatic disease.

Administration of a compound described herein may be achieved in any suitable manner including, by way of non-limiting example, by oral, enteric, parenteral (e.g., intravenous, subcutaneous, intramuscular), intranasal, buccal, topical, rectal, or transdermal administration routes. Any compound or composition described herein may be administered in a method or formulation appropriate to treat a newborn or an infant. Any compound or composition described herein may be administered in an oral formulation (e.g., solid or liquid) to treat a newborn or an infant. Any compound or composition described herein may be administered prior to ingestion of food, with food or after ingestion of food.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, the individual's health status, weight, and response to the drugs, and the judgment of the treating physician.

In prophylactic applications, compounds or compositions containing compounds described herein may be administered to an individual susceptible to or otherwise at risk of a particular disease, disorder or condition. In certain embodiments of this use, the precise amounts of compound administered depend on the individual's state of health, weight, and the like. Furthermore, in some instances, when a compound or composition described herein is administered to an individual, effective amounts for this use depend on the severity and course of the disease, disorder or condition, previous therapy, the individual's health status and response to the drugs, and the judgment of the treating physician.

In certain embodiments of the methods of the present invention, wherein following administration of a selected dose of a compound or composition described herein, an individual's condition does not improve, upon the doctor's discretion the administration of a compound or composition described herein is optionally administered chronically, that is, for an extended period of time, including throughout the duration of the individual's life in order to ameliorate or otherwise control or limit the symptoms of the individual's disorder, disease or condition.

In certain embodiments of the methods of the present invention, an effective amount of a given agent varies depending upon one or more of a number of factors such as the particular compound, disease or condition and its severity, the identity (e.g., weight) of the subject or host in need of treatment, and is determined according to the particular circumstances surrounding the case, including, e.g., the specific agent being administered, the route of administration, the condition being treated, and the subject or host being treated. In some embodiments, doses administered include those up to the maximum tolerable dose. In some embodiments, doses administered include those up to the maximum tolerable dose by a newborn or an infant.

In various embodiments of the methods of the present invention, a desired dose is conveniently presented in a single dose or in divided doses administered simultaneously (or over a short period of time) or at appropriate intervals, for example as two, three, four or more sub-doses per day. In various embodiments, a single dose of an IBAT inhibitor is administered every 6 hours, every 12 hours, every 24 hours, every 48 hours, every 72 hours, every 96 hours, every 5 days, every 6 days, or once a week. In some embodiments the total single dose of an IBAT inhibitor is in a range described below.

In various embodiments of methods of the present invention, in the case wherein the patient's status does improve, upon the doctor's discretion an IBAT inhibitor is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100% of the original dose, including, by way of example only, 10%, 15%, 20%, 25%, 30%, 35%, 40%, 45%, 50%, 55%, 60%, 65%, 70%, 75%, 80%, 85%, 90%, 95%, or 100% of the original dose. In some embodiments the total single dose of an MAT inhibitor is in a range described below.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are optionally determined by pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are prefer ed. In certain embodiments, data obtained from cell culture assays and animal studies are used in formulating a range of dosage for use in human. In specific embodiments, the dosage of compounds described herein lies within a range of circulating concentrations that include the ED₅₀ with minimal toxicity. The dosage optionally varies within this range depending upon the dosage form employed and the route of administration utilized.

In certain embodiments, the composition used or administered comprises an absorption inhibitor, a carrier, and one or more of a cholesterol absorption inhibitor, an enteroendocrine peptide, a peptidase inhibitor, a spreading agent, and a wetting agent.

In some embodiments of methods of the present invention, the composition used to prepare an oral dosage form or administered orally comprises an absorption inhibitor, an orally suitable carrier, an optional cholesterol absorption inhibitor, an optional enteroendocrine peptide, an optional peptidase inhibitor, an optional spreading agent, and an optional wetting agent. In certain embodiments, the orally administered compositions evoke an anorectal response. In specific embodiments, the anorectal response is an increase in secretion of one or more enteroendocrine by cells in the colon and/or rectum (e.g., in L-cells the epithelial layer of the colon, ileum, rectum, or a combination thereof). In some embodiments, the anorectal response persists for at least 1, 2, 3, 4, 5, 6, 7, 8, 9, 10, 11, 12, 13, 14, 15, 16, 17, 18, 19, 20, 21, 22, 23 or 24 hours. In other embodiments the anorectal response persists for a period between 24 hours and 48 hours, while in other embodiments the anorectal response persists for persists for a period greater than 48 hours.

### Dosages

In various embodiments the IBAT inhibitor is maralixibat or volixibat, or a pharmaceutically acceptable salt thereof.

In various embodiments the IBAT inhibitor is administered to a subject before ingestion of food.

In various embodiments, efficacy and safety of IBAT inhibitor administration to the patient is monitored by measuring serum levels of 7α-hydroxy-4-cholesten-3-one (7αC4), sBA concentration, a ratio of 7αC4 to sBA (7αC4:sBA), serum conjugated bilirubin concentration, serum autotaxin concentration, serum bilirubin concentration, serum total cholesterol concentration, serum LDL-C concentration, serum ALT concentration, serum AST concentration, or a combination thereof. In various embodiments, efficacy of IBAT inhibitor administration is measured by monitoring observer-reported itch reported outcome (ITCHRO(OBS)) score, a HRQoL (e.g., PedsQL) score, a CSS score, a xanthoma score, a height Z-score, a weight Z-score, or various combinations thereof. In various embodiments, the method includes monitoring serum levels of 7α-hydroxy-4-cholesten-3-one (7αC4), sBA concentration, a ratio of 7αC4 to sBA (7αC4:sBA), serum conjugated bilirubin concentration, serum total cholesterol concentration, serum LDL-C concentration, serum autotaxin concentration, serum bilirubin concentration, serum ALT concentration, serum AST concentration, or a combination thereof. In various embodiments, the method includes monitoring observer-reported itch reported outcome (ITCHRO(OBS)) score, a weight Z-score, a HRQoL (e.g., PedsQL) score, a xanthoma score, a CSS score, a height Z-score, or various combinations thereof.

In some embodiments, the IBAT inhibitor is administered at a dose of about or at least about 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 100 μg/kg, 140 μg/kg, 150 μg/kg, 200 μg/kg, 240 μg/kg, 250 μg/kg, 280 μg/kg, 300 μg/kg, 360 μg/kg, 380 μg/kg, 400 μg/kg, 500 μg/kg, 560 μg/kg, 600 μg/kg, 700 μg/kg, 800 μg/kg, 880 μg/kg, 900 μg/kg, 1,000 μg/kg, 1,100 μg/kg, 1,200 μg/kg, 1,300 μg/kg, 1,400 μg/kg, 1500 μg/kg, 1,600 μg/kg, 1,700 μg/kg, 1,800 μg/kg, 1,900 μg/kg, or 2,000 μg/kg. In various embodiments, the IBAT inhibitor is administered at a dose not exceeding about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 100 μg/kg, 140 μg/kg, 150 μg/kg, 200 μg/kg, 240 μg/kg, 250 μg/kg, 280 μg/kg, 300 μg/kg, 360 μg/kg, 380 μg/kg, 400 μg/kg, 500 μg/kg, 560 μg/kg, 600 μg/kg, 700 μg/kg, 800 μg/kg, 880 μg/kg, 900 μg/kg, 1,000 μg/kg, 1,100 μg/kg, 1,200 μg/kg, 1,300 μg/kg, 1,400 μg/kg, 1,500 μg/kg, 1,600 μg/kg, 1,700 μg/kg, 1,800 μg/kg, 1,900 μg/kg, 2,000, or 2,100 μg/kg. In various embodiments, the IBAT inhibitor is administered at a dose of about or of at least about 0.5 mg/day, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 30 mg/day, 40 mg/day, 50 mg/day, 60 mg/day, 70 mg/day, 80 mg/day, 90 mg/day, 100 mg/day, 150 mg/day, 200 mg/day, 300 mg/day, 500 mg/day, 600 mg/day, 700 mg/day, 800 mg/day, 900 mg/day, 1000 mg/day. In various embodiments, the IBAT inhibitor is administered at a dose of not more than about 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 30 mg/day, 40 mg/day, 50 mg/day, 60 mg/day, 70 mg/day, 80 mg/day, 90 mg/day, 100 mg/day, 150 mg/day, 200 mg/day, 300 mg/day, 500 mg/day, 600 mg/day, 700 mg/day, 800 mg/day, 900 mg/day, 1,000 mg/day, 1,100 mg/day.

In some embodiments, the IBAT inhibitor is administered at a dose of from about 140 μg/kg/day to about 1400 μg/kg/day. In various embodiments, the IBAT inhibitor is administered at a dose of about or at least about 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day 10 μg/kg/day, 15 μg/kg/day, 20 μg/kg/day, 25 μg/kg/day, 30 μg/kg/day, 35 μg/kg/day, 40 μg/kg/day, 45 μg/kg/day, 50 μg/kg/day, 100 μg/kg/day, 140 μg/kg/day, 150 μg/kg/day, 200 μg/kg/day, 240 μg/kg/day, 280 μg/kg/day, 300 μg/kg/day, 250 μg/kg/day, 280 μg/kg/day, 300 μg/kg/day, 360 μg/kg/day, 380 μg/kg/day, 400 μg/kg/day, 500 μg/kg/day, 560 μg/kg/day, 600 μg/kg/day, 700 μg/kg/day, 800 μg/kg/day, 880 μg/kg, 900 μg/kg/day, 1,000 μg/kg/day, 1,100 μg/kg/day, 1,200 μg/kg/day, or 1,300 μg/kg/day. In various embodiments, the IBAT inhibitor is administered at a dose not exceeding about 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day 10 μg/kg/day, 15 μg/kg/day, 20 μg/kg/day, 25 μg/kg/day, 30 μg/kg/day, 35 μg/kg/day, 40 μg/kg/day, 45 μg/kg/day, 50 μg/kg/day, 100 μg/kg/day, 140 μg/kg/day, 150 μg/kg/day, 200 μg/kg/day, 240 μg/kg/day, 280 μg/kg/day, 300 μg/kg/day, 250 μg/kg/day, 280 μg/kg/day, 300 μg/kg/day, 360 μg/kg/day, 380 μg/kg/day, 400 μg/kg/day, 500 μg/kg/day, 560 μg/kg/day, 600 μg/kg/day, 700 μg/kg/day, 800 μg/kg/day, 880 μg/kg/day, 900 μg/kg/day, 1,000 μg/kg/day, 1,100 μg/kg/day, 1,200 μg/kg/day, 1,300 μg/kg/day, or 1,400 μg/kg/day. In various embodiments, the IBAT inhibitor is administered at a dose of from about 0.5 μg/kg/day to about 500 μg/kg/day, from about 0.5 μg/kg/day to about 250 μg/kg/day, from about 1 μg/kg/day to about 100 μg/kg/day, from about 10 μg/kg/day to about 50 μg/kg/day, from about 10 μg/kg/day to about 100 μg/kg/day, from about 0.5 μg/kg/day to about 2000 μg/kg/day, from about 280 μg/kg/day to about 1400 μg/kg/day, from about 420 μg/kg/day to about 1400 μg/kg/day, from about 250 to about 550 μg/kg/day, from about 560 μg/kg/day to about 1400 μg/kg/day, from 700 μg/kg/day to about 1400 μg/kg/day, from about 560 μg/kg/day to about 1200 μg/kg/day, from about 700 μg/kg/day to about 1200 μg/kg/day, from about 560 μg/kg/day to about 1000 μg/kg/day, from about 700 μg/kg/day to about 1000 μg/kg/day, from about 800 μg/kg/day to about 1000 μg/kg/day, from about 200 μg/kg/day to about 600 μg/kg/day, from about 300 μg/kg/day to about 600 μg/kg/day, from about 400 μg/kg/day to about 500 μg/kg/day, from about 400 μg/kg/day to about 600 μg/kg/day, from about 400 μg/kg/day to about 700 μg/kg/day, from about 400 μg/kg/day to about 800 μg/kg/day, from about 500 μg/kg/day to about 800 μg/kg/day, from about 500 μg/kg/day to about 900 μg/kg/day, from about 600 μg/kg/day to about 900 μg/kg/day, from about 700 μg/kg/day to about 900 μg/kg/day, from about 200 μg/kg/day to about 600 μg/kg/day, from about 800 μg/kg/day to about 900 μg/kg/day, from about 100 μg/kg/day to about 1500 μg/kg/day, from about 300 μg/kg/day to about 2,000 μg/kg/day, or from about 400 μg/kg/day to about 2000 μg/kg/day.

In some embodiments, the IBAT inhibitor is administered at a dose of from about 30 μg/kg to about 1400 μg/kg per dose. In some embodiments, the IBAT inhibitor is administered at a dose of from about 0.5 μg/kg to about 2000 μg/kg per dose, from about 0.5 μg/kg to about 1500 μg/kg per dose, from about 100 μg/kg to about 700 μg/kg per dose, from about 5 μg/kg to about 100 μg/kg per dose, from about 10 μg/kg to about 500 μg/kg per dose, from about 50 μg/kg to about 1400 μg/kg per dose, from about 300 μg/kg to about 2,000 μg/kg per dose, from about 60 μg/kg to about 1200 μg/kg per dose, from about 70 μg/kg to about 1000 μg/kg per dose, from about 70 μg/kg to about 700 μg/kg per dose, from 80 μg/kg to about 1000 μg/kg per dose, from 80 μg/kg to about 800 μg/kg per dose, from 100 μg/kg to about 800 μg/kg per dose, from 100 μg/kg to about 600 μg/kg per dose, from 150 μg/kg to about 700 μg/kg per dose, from 150 μg/kg to about 500 μg/kg per dose, from 200 μg/kg to about 400 μg/kg per dose, from 200 μg/kg to about 300 μg/kg per dose, or from 300 μg/kg to about 400 μg/kg per dose.

In some embodiments, the IBAT inhibitor is administered at a dose of from about 0.5 mg/day to about 550 mg/day. In various embodiments, the IBAT inhibitor is administered at a dose of from about 1 mg/day to about 500 mg/day, from about 1 mg/day to about 300 mg/day, from about 1 mg/day to about 200 mg/day, from about 2 mg/day to about 300 mg/day, from about 2 mg/day to about 200 mg/day, from about 4 mg/day to about 300 mg/day, from about 4 mg/day to about 200 mg/day, from about 4 mg/day to about 150 mg/day, from about 5 mg/day to about 150 mg/day, from about 5 mg/day to about 100 mg/day, from about 5 mg/day to about 80 mg/day, from about 5 mg/day to about 50 mg/day, from about 5 mg/day to about 40 mg/day, from about 5 mg/day to about 30 mg/day, from about 5 mg/day to about 20 mg/day, from about 5 mg/day to about 15 mg/day, from about 10 mg/day to about 100 mg/day, from about 10 mg/day to about 80 mg/day, from about 10 mg/day to about 50 mg/day, from about 10 mg/day to about 40 mg/day, from about 10 mg/day to about 20 mg/day, from about 20 mg/day to about 100 mg/day, from about 20 mg/day to about 80 mg/day, from about 20 mg/day to about 50 mg/day, or from about 20 mg/day to about 40 mg/day, or from about 20 mg/day to about 30 mg/day.

In some embodiments, the IBAT inhibitor is administered twice daily (BID) in an amount of about 200 μg/kg to about 400 μg/kg per dose. In some embodiments, the IBAT inhibitor is administered in an amount of about 280 μg/kg/day to about 1400 μg/kg/day. In some embodiments, the IBAT inhibitor is administered in an amount of about 400 μg/kg/day to about 800 μg/kg/day. In some embodiments, the IBAT inhibitor is administered in an amount of about 20 mg/day to about 50 mg/day. In some embodiments, the IBAT inhibitor is administered in an amount of from about 5 mg/day to about 15 mg/day. In some embodiments, the IBAT inhibitor is administered in an amount of from about 560 μg/kg/day to about 1,400 μg/kg/day. In some embodiments, the IBAT inhibitor is administered in an amount of from about 700 μg/kg/day to about 1,400 μg/kg/day. In some embodiments, the IBAT inhibitor is administered in an amount of from about 400 μg/kg/day to about 800 μg/kg/day. In some embodiments, the IBAT inhibitor is administered in an amount of from about 700 μg/kg/day to about 900 μg/kg/day. In some embodiments, the IBAT inhibitor is administered in an amount of from about 560 μg/kg/day to about 1400 μg/kg/day. In some embodiments, the IBAT inhibitor is administered in an amount from 700 μg/kg/day to about 1400 μg/kg/day. In some embodiments, the IBAT inhibitor is administered in an amount of from about 200 μg/kg/day to about 600 μg/kg/day. In some embodiments, the IBAT inhibitor is administered in an amount of from about 400 μg/kg/day to about 600 μg/kg/day.

In various embodiments, the dose of the IBAT inhibitor is a first dose level. In various embodiments, the dose of the IBAT inhibitor is a second dose level. In some embodiments, the second dose level is greater than the first dose level. In some embodiments, the second dose level is about or at least about 1.5, 2, 3, 4, 5, 6, 7, 8, 9, 10, 15, 20, 30, 40, 50, 60, 70, 80, 90 or 100 times or fold greater than the first dose level. In some embodiments, the second dose level is not in excess of about 1.5, 2, 3, 4, 5, 6, 7, 8, 9, 10, 15, 20, 30, 40, 50, 60, 70, 80, 90, 100, or 150 times or fold greater than the first dose level.

In various embodiments, the IBAT inhibitor is administered once daily (QD) at one of the above doses or within one of the above dose ranges. In various embodiments, the IBAT inhibitor is administered twice daily (BID) at one of the above doses or within one of the above dose ranges. In various embodiments, an IBAT inhibitor dose is administered daily, every other day, twice a week, or once a week.

In various embodiments, the IBAT inhibitor is administered regularly for a period of about or of at least about 1, 2, 3, 4, 5, 6, 7, 8, 9, 10, 15, 20, 30, 40, 48, 50, 75, 100, 150, 200, 250, 300, 350, 400, 450, 500, 600, 700, or 800 weeks. In various embodiments, the IBAT inhibitor is administered for not more than about 1, 2, 3, 4, 5, 6, 7, 8, 9, 10, 15, 20, 30, 40, 48, 50, 75, 100, 150, 200, 250, 300, 350, 400, 450, 500, 600, 700, 800, or 1000 weeks. In various embodiments, the IBAT inhibitor is administered regularly for a period of about or of at least about 0.5, 1, 1.5, 2, 3, 4, 5, 6, 7, 8, 9, or 10 years. In various embodiments, the IBAT inhibitor is administered regularly for a period not in excess of about 0.5, 1, 1.5, 2, 3, 4, 5, 6, 7, 8, 9, 10, or 15 years.

### Reduction in Symptoms or a Change in a Disease-Relevant Laboratory Measures of Cholestatic Liver Disease

In various embodiments of the above methods of the invention, administration of the IBAT inhibitor results in a reduction in a symptom or a change in a disease-relevant laboratory measure of the cholestatic liver disease (i.e., improvement in the patient's condition) that is maintained for about or for at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years. In various embodiments, the reduction in the symptom or a change in a disease-relevant laboratory measure comprises a reduction in sBA concentration, an increase in serum 7αC4 concentration, an increase in the 7αC4:sBA ratio, an increase in fBA excretion, a reduction in pruritis, a decrease in serum total cholesterol concentration, a decrease in serum LDL-C cholesterol concentration, a reduction in ALT levels, an increase in a quality of life inventory score, an increase in a quality of life inventory score related to fatigue, a reduction in a xanthoma score, a reduction in serum autotaxin concentration, an increase in growth, or a combination thereof. In various embodiments, the reduction in the symptom or a change in a disease-relevant laboratory measure is determined relative to a baseline level. That is, the reduction in the symptom or a change in a disease-relevant laboratory measure is determined relative to a measurement of the symptom or a change in a disease-relevant laboratory measure prior to 1) changing a dose level of the IBAT inhibitor administered to the patient, 2) changing a dosing regimen followed for the patient, 3) commencing administration of the IBAT inhibitor, or 4) any other of various alterations made with the intention of reducing the symptom or a change in a disease-relevant laboratory measure in the patient. In various embodiments, the reduction in symptom or a change in a disease-relevant laboratory measure is a statistically significant reduction.

In various embodiments, the reduction in a symptom or a change in a disease-relevant laboratory measure of the cholestatic liver disease is measured as a progressive decrease in the symptom or a change in a disease-relevant laboratory measure for about or for at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years.

In some embodiments, the patient is the pediatric patient and the reduction in symptom or a change in a disease-relevant laboratory measure comprises an increase or improvement in growth. In some embodiments, the increase in growth is measured relative to baseline. In various embodiments, increase in growth is measured as an increase in height Z-score or in weight Z-score. In various embodiments, the increase in height Z-score or in weight Z-score is statistically significant. In various embodiments, the height Z-score, the weight Z-score, or both is increased by at least 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29 0.3, 0.31, 0.32, 0.33, 0.34, 0.35, 0.36, 0.37, 0.38, 0.39 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.7, 0.8, or 0.9 relative to baseline. In some embodiments, the height Z-score, the weight Z-score, or both progressively increases during administration of the IBAT inhibitor for a period of about or of at least about 1, 2, 3, 4, 5, 6, 7, 8, 9, 10, 20, 30, 40, 48, 50, 60, 70, or 72 weeks.

In various embodiments, the administration of the IBAT inhibitor results in an increase in serum 7αC4 concentration. In various embodiments, the serum 7αC4 concentration is increased by about or at least about 1.5, 2, 3, 4, 5, 6, 7, 8, 9, 10, 15, 20, 30, 40, 50, 60, 70, 80, 90, 100, 200, 300, 400, or 500 times or fold relative to baseline. In various embodiments the serum 7αC4 concentration is increased about or at least about 10%, 20%, 30%, 40%, 50%, 60%, 70%, 80%, 90%, 100%, 150%, 200%, 300%, 400%, 500%, 600%, 700%, 800%, 900%, 1,000%, or 10,000% relative to baseline.

In various embodiments, the administration of the IBAT inhibitor results in an increase in the 7αC4:sBA ratio to about or by at least about 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold relative to baseline.

In various embodiments, the administration of the IBAT inhibitor results in an increase in fBA excretion. In some embodiments, the administration of the IBAT inhibitor results in an increase in fBA excretion of about or of at least about 100%, 110%, 115%, 120%, 130%, 150%, 200%, 250%, 275%, 300%, 400%, 500%, 600%, 700%, 800%, 1,000%, 5,000%, 10,000% or 15,000% relative to baseline. In various embodiments, fBA excretion is increased by about or by at least about 1, 1.5, 2, 3, 4, 5, 6, 7, 8, 9, 10, 20, 30, 40, 50, 60, 70, 80, 90, or 100 fold or times relative to baseline. In some embodiments, fBA excretion is increased by about or by at least about 100 μmol, 150 μmol, 200 μmol, 250 mol, 300 mol, 400 mol, 500 mol, 600 mol, 700 mol, 800 mol, 900 mol, 1,000 mol, or 1,500 mol relative to baseline. In various embodiments, administration of the IBAT inhibitor results in a dose-dependent increase in fBA excretion so that administration of a higher dose of the IBAT inhibitor results in a corresponding higher level of fBA excretion. In various embodiments, the IBAT inhibitor is administered at a dose sufficient to result in an increase in bile acid secretion relative to baseline of at least about or of about 1, 2, 3, 4, 5, 6, 7, 8, 9, 10, 20, 30, 40, 50, 60, 70, 80, 90, or 100 fold or times relative to baseline.

In various embodiments, the administration of the IBAT inhibitor results in a decrease in sBA concentration of about or of at least about 5%, 10%, 15%, 20%, 25%, 30%, 31%, 35%, 40%, 45%, 50%, 55%, 57%, 60%, 65%, 70%, 75%, 80%, 85%, 90%, or 95% relative to baseline.

In some embodiments, the administration of the IBAT inhibitor results in a reduction in severity of pruritus. In various embodiments, the severity of pruritus is measured using an ITCHRO(OBS) score, an ITCHRO score, a CSS score, or a combination thereof. In various embodiments, the administration of the IBAT inhibitor results in a reduction in the ITCHRO(OBS) score on a scale of 1 to 4 of about or of at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, or 3 relative to baseline. In various embodiments, the administration of the IBAT inhibitor results in a reduction in the ITCHRO score on a scale of 1 to 10 of about or of at least about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, or 10. In various embodiments, the administration of the IBAT inhibitor results in a reduction of the ITCHRO(OBS) score, the ITCHRO score, or both to zero. In various embodiments, the administration of the IBAT inhibitor results in a reduction of the ITCHRO(OBS) score or ITCHRO score to 1.0 or lower. In various embodiments, the administration of the IBAT inhibitor results in a reduction of the CSS score by about of at least about 0.1, 0.2, 0.3, 0.4, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, or 3 relative to baseline. In various embodiments, the administration of the IBAT inhibitor results in a reduction of the CSS score to zero. In various embodiments, the administration of the IBAT inhibitor results in a reduction in the CSS score, the ITCHRO(OBS) score, the ITCHRO score, or a combination thereof by about or by at least about 10%, 15%, 20%, 25%, 30%, 35%, 40%, 45%, 50%, 55%, 60%, 65%, 70%, 75%, 80%, 85%, 90%, 95%, or 100% relative to baseline. In various embodiments, a reduced value relative to baseline of the CSS score, the ITCHRO(OBS) score, the ITCHRO score, or a combination thereof is observed on 10%, 20%, 30%, 40%, 50%, 60%, 70%, 75%, 80%, 85%, 90%, 91%, 92%, 93%, 94%, 95%, 96%, 97%, 98%, 99%, or 100% of days.

In some embodiments, patients with a higher baseline ITCHRO(OBS) score demonstrate a greater reduction in the symptom or a change in a disease-relevant laboratory measure than patients having a lower baseline ITCHRO(OBS) score. In some embodiments, patients with a baseline ITCHRO(OBS) score of at least 2, 3, or 4 or an ITCHRO score of at least 4, 5, 6, 7, 8, 9, or 10 have a greater reduction in the symptom or a change in a disease-relevant laboratory measure relative to baseline than a lower reduction in patients having a lower baseline severity of pruritus score. In various embodiments, patients having PSC and baseline ITCHRO scores of at least 4 demonstrate a greater reduction in the symptom or a change in a disease-relevant laboratory measure than patients having a baseline ITCHRO score of less than 4. In various embodiments, the method includes predicting that a patient will have a greater reduction in the symptom or a change in a disease-relevant laboratory measure if a baseline ITCHRO score of the patient is at least 4 as compared to a patient having a baseline ITCHRO score of less than 4. In various embodiments the lower reduction is about or less than about 5%, 10%, 15%, 20%, 25%, 30%, 35%, 40%, 45%, 50%, 55%, or 60% the greater reduction. In various embodiments a difference in the reduction in the symptom or a change in a disease-relevant laboratory measure (i.e., between the greater reduction and the lower reduction) between patients having an ITCHRO score of at least 4 at baseline and patients having an ITCHRO score of less than 4 at baseline is measured at about or at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years following first administration of the IBAT inhibitor at the first dose or at the second dose.

In various embodiments, reduction in severity of pruritus caused by administration of the IBAT inhibitor to the patient is positively correlated with a reduction in sBA concentration in the patient. In various embodiments, a greater reduction in sBA concentration in the patient correlates with a corresponding greater reduction in severity of pruritus.

In various embodiments, the administration of the IBAT inhibitor results in a reduction in serum LDL-C concentration relative to baseline. In some embodiments the serum LDL-C concentration is reduced by about or by at least about 1%, 2%, 3%, 4%, 5%, 10%, 15%, 20%, 25%, 25%, 30%, 35%, 40%, 45%, 50%, 55%, 60%, 65%, 70%, 75%, or 80% relative to baseline.

In some embodiments, the administration of the IBAT inhibitor results in a reduction in serum total cholesterol concentration relative to baseline. In some embodiments, the administration of the IBAT inhibitor results in a reduction in serum LDL-C levels relative to baseline. In some embodiments the serum total cholesterol concentration, the serum LDL-C levels, or both is reduced by about or by at least about 1%, 2%, 3%, 4%, 5%, 10%, 15%, 20%, 25%, 25%, 30%, 35%, 40%, 45%, 50%, 55%, 60%, 65%, 70%, 75%, or 80% relative to baseline. In various embodiments, the administration of the IBAT inhibitor results in a reduction in serum total cholesterol concentration, of serum LDL-C levels, or both of about or of at least about 1 mg/dL, 2 mg/dL, 3 mg/dL, 4 mg/dL, 5 mg/dL, 10 mg/dL, 12.5 mg/dL, 15 mg/dL, 20 mg/dL, 30 mg/dL, 40 mg/dL or 50 mg/dL relative to baseline.

In various embodiments, the administration of the IBAT inhibitor results in a decrease in serum autotaxin concentration. In some embodiments, the administration of the IBAT inhibitor results in a reduction in autotaxin concentration of about or of at least about 5%, 10%, 15%, 20%, 25%, 30%, 35%, 40%, 45%, 50%, 55%, 60%, 65%, 70%, 75%, or 80% relative to baseline.

In various embodiments, administration of the IBAT inhibitor results in an increase in a quality of life inventory score or in a quality of life inventory score related to fatigue. The quality of life inventory score can be a health-related quality of life (HRQoL) score. In some embodiments, the HRQoL score is a PedsQL score. In various embodiments, the administration of the IBAT inhibitor results an increase in the PedsQL score or in a PedsQL score related to fatigue of about or of at least about 5%, 10%, 15%, 20%, 25%, 30%, 45%, or 50% relative to baseline.

In various embodiments, administration of the IBAT inhibitor results in a decrease in a xanthoma score relative to baseline. In some embodiments, the xanthoma score is reduced by about or by at least about 2.5%, 5%, 10%, 15%, 20%, 35%, 30%, 35%, 40%, 45%, 50%, 55%, 60%, 65%, 70%, 75%, 80%, 85%, 90%, 95%, or 100% relative to baseline.

In various embodiments, the administration of the IBAT inhibitor results in the reduction in the symptom or a change in a disease-relevant laboratory measure by about 1 day, 2 days, 3 days, 4 days, 5 days, 6 days, 7 days, 8 days, 9 days, 10 days, 11 days, 12, days, 13 days, 14 days, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year, or 2 years, or 3 years, or 4 years, or 5 years, or 6 years.

In various embodiments, serum bilirubin concentration is at pre-administration baseline levels or at normal levels at about or by about 1 day, 2 days, 3 days, 4 days, 5 days, 6 days, 1 week, 2 weeks, 3 weeks, 4 weeks, 1 month, 5 weeks, 6 weeks, 7 weeks, 8 weeks, 2 months, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year, or 2 years, or 3 years, or 4 years, or 5 years, or 6 years.

In various embodiments, serum ALT concentration is at pre-administration baseline levels or at normal levels at about or by about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year, or 2 years, or 3 years, or 4 years, or 5 years, or 6 years. In some embodiments, the administration of the IBAT inhibitor results in a reduction in ALT levels relative to baseline of about or of at least about 1%, 2%, 3%, 4%, 5%, 6%, 7%, 8%, 9%, 10%, 11%, 12%, 13%, 14%, or 15%.

In various embodiments, serum ALT concentration, serum AST concentration, serum bilirubin concentration, serum conjugated bilirubin concentration, or various combinations thereof are within normal range or at pre-administration baseline levels at about or by about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year, or 2 years, or 3 years, or 4 years, or 5 years, or 6 years. In various embodiments, the administration of the IBAT inhibitor does not result in a statistically significant change from baseline in serum bilirubin concentration, serum AST concentration, serum ALT concentration, serum alkaline phosphatase concentration, or some combination thereof for a period of at least about or of about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year, or 2 years, or 3 years, or 4 years, or 5 years, or 6 years. In various embodiments, for adult patients with an ITCHRO score of at least 4 at baseline, the administration of the IBAT inhibitor does not result in a significant change from baseline in serum conjugated bilirubin concentration for a period of at least about or of about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year, or 2 years, or 3 years, or 4 years, or 5 years, or 6 years.

In various embodiments of the above methods of the invention, administration of the IBAT inhibitor results in reduction, prevention, amelioration, or elimination of one or more side effects associated with administration of the IBAT inhibitor in a subject in need thereof. In various embodiments, the frequency and/or severity of side effects is reduced as compared to the side effects when the IBAT inhibitor is administered after ingestion of food, at the same time as food, or mixed with food. In various embodiments, the one of more side effects is diarrhea, loose stools, nausea, gastrointestinal pain, abdominal pain, cramping, anorectal discomfort, or a combination thereof.

### Dose Modulation

In various embodiments, the method includes modulating a dosage of the IBAT inhibitor administered to the patient. The modulation includes determining the 7αC4:sBA ratio for the patient at a baseline (e.g., prior to administration of the IBAT inhibitor or prior to modulating (e.g., increasing) a dosage of the IBAT inhibitor), and further determining the 7αC4:sBA ratio after administering the IBAT inhibitor at a first dose or modulating (e.g., increasing) a dosage amount of the IBAT inhibitor to a second dose. If the 7αC4:sBA ratio does not increase by at least 1, 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold from baseline, the dose of the IBAT inhibitor is increased until the ratio increases at least about 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold relative to baseline. In various embodiments, the dose of the IBAT inhibitor is increased or decreased to achieve and maintain a particular 7αC4:sBA ratio.

In various embodiments, the modulating includes increasing a dose of the IBAT inhibitor from a first dose level to a second dose level greater than the first dose level if the 7αC4:sBA ratio initially increases by at least 1, 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold from baseline and then begins to decrease or decreases to less than 1, 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold or greater higher than baseline. The dose level is increased until the 7αC4:sBA ratio increases to at least 1, 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold from the baseline.

In some embodiments, the modulation includes administering a first dose of the IBAT inhibitor to the patient. If the 7αC4:sBA ratio does not increase or increase by at least 1, 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold from baseline, the patient is then administered a second dose of the IBAT inhibitor higher than the first dose. The dose administered to the patient continues to be increased until the 7αC4:sBA ratio increases by at least 1, 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold from baseline.

In various embodiments, the 7αC4:sBA ratio is measured about daily, bi-weekly, weekly, bi-monthly, monthly, every two months, every three months, every four months, every five months, every six months, or annually, and the dose of the IBAT inhibitor is modulated as necessary each time the ratio is measured.

### Pharmaceutical Compositions

In some embodiments, the IBAT inhibitor is administered as a pharmaceutical composition comprising an IBAT inhibitor (the composition or the pharmaceutical composition). Any composition described herein can be formulated for ileal, rectal and/or colonic delivery. In more specific embodiments, the composition is formulated for non-systemic or local delivery to the rectum and/or colon. It is to be understood that, as used herein, delivery to the colon includes delivery to sigmoid colon, transverse colon, and/or ascending colon. In still more specific embodiments, the composition is formulated for non-systemic or local delivery to the rectum and/or colon is administered rectally. In other specific embodiments, the composition is formulated for non-systemic or local delivery to the rectum and/or colon is administered orally.

Provided herein, in certain embodiments, is a pharmaceutical composition comprising a therapeutically effective amount of any compound described herein. In certain instances, the pharmaceutical composition comprises an IBAT inhibitor (e.g., any IBAT inhibitor described herein).

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Mareel Decker, New York, N.Y., 1980; and Pharmaceutical Dosage Forms and Drug Delivery Systems, Seventh Ed. (Lippincott Williams & Wilkins1999), all of which references are incorporated herein in their entirety for all purposes.

A pharmaceutical composition, as used herein, refers to a mixture of a compound described herein, with other chemical components, such as carriers, stabilizers, diluents, dispersing agents, suspending agents, thickening agents, and/or excipients. In certain instances, the pharmaceutical composition facilitates administration of the compound to an individual or cell. In certain embodiments of practicing the methods of treatment or use provided herein, therapeutically effective amounts of compounds described herein are administered in a pharmaceutical composition to an individual having a disease, disorder, or condition to be treated. In specific embodiments, the individual is a human. As discussed herein, the compounds described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes.

In certain embodiments, a pharmaceutical compositions described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” includes, in some embodiments, a pharmaceutically acceptable excipient and is selected on the basis of compatibility with compounds described herein, such as, compounds of any of Formula I-VI, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Mareel Decker, New York, N.Y., 1980; and Pharmaceutical Dosage Forms and Drug Delivery Systems, Seventh Ed. (Lippincott Williams & Wilkins 1999), all of which references are incorporated herein in their entirety for all purposes.

Moreover, in certain embodiments, the pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In some embodiments, provided herein is a composition comprising an enteroendocrine peptide secretion enhancing agent and, optionally, a pharmaceutically acceptable carrier for alleviating symptoms of cholestasis or a cholestatic liver disease in an individual.

In certain embodiments, the composition comprises an enteroendocrine peptide secretion enhancing agent and an absorption inhibitor. In specific embodiments, the absorption inhibitor is an inhibitor that inhibits the absorption of the (or at least one of the) specific enteroendocrine peptide secretion enhancing agent with which it is combined. In some embodiments, the composition comprises an enteroendocrine peptide secretion enhancing agent, an absorption inhibitor and a carrier (e.g., an orally suitable carrier or a rectally suitable carrier, depending on the mode of intended administration). In certain embodiments, the composition comprises an enteroendocrine peptide secretion enhancing agent, an absorption inhibitor, a carrier, and one or more of a cholesterol absorption inhibitor, an enteroendocrine peptide, a peptidase inhibitor, a spreading agent, and a wetting agent.

In other embodiments, the compositions described herein are administered orally for non-systemic delivery of the IBAT inhibitor to the rectum and/or colon, including the sigmoid colon, transverse colon, and/or ascending colon. In specific embodiments, compositions formulated for oral administration are, by way of non-limiting example, enterically coated or formulated oral dosage forms, such as, tablets and/or capsules.

### Absorption Inhibitors

In certain embodiments, the composition described herein as being formulated for the non-systemic delivery of IBAT inhibitor further includes an absorption inhibitor. As used herein, an absorption inhibitor includes an agent or group of agents that inhibit absorption of a bile acid/salt.

Suitable bile acid absorption inhibitors (also described herein as absorption inhibiting agents) may include, by way of non-limiting example, anionic exchange matrices, polyamines, quaternary amine containing polymers, quaternary ammonium salts, polyallylamine polymers and copolymers, colesevelam, colesevelam hydrochloride, CholestaGel (N,N,N-trimethyl-6-(2-propenylamino)-1-hexanaminium chloride polymer with (chloromethyl)oxirane, 2-propen-1-amine and N-2-propenyl-1-decanamine hydrochloride), cyclodextrins, chitosan, chitosan derivatives, carbohydrates which bind bile acids, lipids which bind bile acids, proteins and proteinaceous materials which bind bile acids, and antibodies and albumins which bind bile acids. Suitable cyclodextrins include those that bind bile acids/salts such as, by way of non-limiting example, β-cyclodextrin and hydroxypropyl-β-cyclodextrin. Suitable proteins, include those that bind bile acids/salts such as, by way of non-limiting example, bovine serum albumin, egg albumin, casein, α-acid glycoprotein, gelatin, soy proteins, peanut proteins, almond proteins, and wheat vegetable proteins.

In certain embodiments the absorption inhibitor is cholestyramine. In specific embodiments, cholestyramine is combined with a bile acid. Cholestyramine, an ion exchange resin, is a styrene polymer containing quaternary ammonium groups crosslinked by divinylbenzene. In other embodiments, the absorption inhibitor is colestipol. In specific embodiments, colestipol is combined with a bile acid. Colestipol, an ion exchange resin, is a copolymer of diethylenetriamine and 1-chloro-2,3-epoxypropane.

In certain embodiments of the compositions and methods described herein the IBAT inhibitor is linked to an absorption inhibitor, while in other embodiments the IBAT inhibitor and the absorption inhibitor are separate molecular entities.

### Cholesterol Absorption Inhibitors

In certain embodiments, a composition described herein optionally includes at least one cholesterol absorption inhibitor. Suitable cholesterol absorption inhibitors include, by way of non-limiting example, ezetimibe (SCH 58235), ezetimibe analogs, ACT inhibitors, stigmastanyl phosphorylcholine, stigmastanyl phosphorylcholine analogues, β-lactam cholesterol absorption inhibitors, sulfate polysaccharides, neomycin, plant sponins, plant sterols, phytostanol preparation FM-VP4, Sitostanol, β-sitosterol, acyl-CoA:cholesterol-O-acyltransferase (ACAT) inhibitors, Avasimibe, Implitapide, steroidal glycosides and the like. Suitable enzetimibe analogs include, by way of non-limiting example, SCH 48461, SCH 58053 and the like. Suitable ACT inhibitors include, by way of non-limiting example, trimethoxy fatty acid anilides such as Cl-976, 3-[decyldimethylsilyl]-N-[2-(4-methylphenyl)-1-phenylethyl]-propanamide, melinamide and the like. β-lactam cholesterol absorption inhibitors include, by way of non-limiting example, βR-4S)-1,4-bis-(4-methoxyphenyl)-3-β-phenylpropyl)-2-azetidinone and the like.

### Peptidase Inhibitors

In some embodiments, the compositions described herein optionally include at least one peptidase inhibitor. Such peptidase inhibitors include, but are not limited to, dipeptidyl peptidase-4 inhibitors (DPP-4), neutral endopeptidase inhibitors, and converting enzyme inhibitors. Suitable dipeptidyl peptidase-4 inhibitors (DPP-4) include, by way of non-limiting example, Vildaglipti, 2.S)-1-{2-[β-hydroxy-1-adamantyl)amino]acetyl}pyrrolidine-2-carbonitrile, Sitagliptin, βR)-3-amino-1-[9-(trifluoromethyl)-1,4,7,8-tetrazabicyclo[4.3.0]nona-6,8-d ien-4-yl]-4-(2,4,5-trifluorophenyl)butan-1-one, Saxagliptin, and (1S,3S,5S)-2-[(25)-2-amino-2-β-hydroxy-1-adamantyl)acetyl]-2-azabicyclo[3.1.0]hexane-3-carbonitrile. Such neutral endopeptidase inhibitors include, but are not limited to, Candoxatrilat and Ecadotril.

### Spreading Agents/Wetting Agents

In certain embodiments, the composition described herein optionally comprises a spreading agent. In some embodiments, a spreading agent is utilized to improve spreading of the composition in the colon and/or rectum. Suitable spreading agents include, by way of non-limiting example, hydroxyethylcellulose, hydroxypropymethyl cellulose, polyethylene glycol, colloidal silicon dioxide, propylene glycol, cyclodextrins, microcrystalline cellulose, polyvinylpyrrolidone, polyoxyethylated glycerides, polycarbophil, di-n-octyl ethers, Cetiol™OE, fatty alcohol polyalkylene glycol ethers, Aethoxal™B), 2-ethylhexyl palmitate, Cegesoft™C 24), and isopropyl fatty acid esters.

In some embodiments, the compositions described herein optionally comprise a wetting agent. In some embodiments, a wetting agent is utilized to improve wettability of the composition in the colon and rectum. Suitable wetting agents include, by way of non-limiting example, surfactants. In some embodiments, surfactants are selected from, by way of non-limiting example, polysorbate (e.g., 20 or 80), stearyl hetanoate, caprylic/capric fatty acid esters of saturated fatty alcohols of chain length C₁₂-C₁₈, isostearyl diglycerol isostearic acid, sodium dodecyl sulphate, isopropyl myristate, isopropyl palmitate, and isopropyl myristate/isopropyl stearate/isopropyl palmitate mixture.

### Vitamins

In some embodiments, the methods provided herein further comprise administering one or more vitamins.

In some embodiments, the vitamin is vitamin A, B1, B2, B3, B5, B6, B7, B9, B12, C, D, E, K, folic acid, pantothenic acid, niacin, riboflavin, thiamine, retinol, beta carotene, pyridoxine, ascorbic acid, cholecalciferol, cyanocobalamin, tocopherols, phylloquinone, menaquinone.

In some embodiments, the vitamin is a fat-soluble vitamin such as vitamin A, D, E, K, retinol, beta carotene, cholecalciferol, tocopherols, phylloquinone. In a preferred embodiment, the fat-soluble vitamin is tocopherol polyethylene glycol succinate (TPGS).

### Bile Acid Sequestrants/Binders

In some embodiments, a labile bile acid sequestrant is an enzyme dependent bile acid sequestrant. In certain embodiments, the enzyme is a bacterial enzyme. In some embodiments, the enzyme is a bacterial enzyme found in high concentration in human colon or rectum relative to the concentration found in the small intestine. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

In certain embodiments, a labile bile acid sequestrant is a time-dependent bile acid sequestrant. In some embodiments, a labile bile acid sequestrant releases a bile acid or is degraded after 1, 2, 3, 4, 5, 6, 7, 8, 9, or 10 seconds of sequestration. In some embodiments, a labile bile acid sequestrant releases a bile acid or is degraded after 15, 20, 25, 30, 35, 40, 45, 50, or 55 seconds of sequestration. In some embodiments, a labile bile acid sequestrant releases a bile acid or is degraded after 1, 2, 3, 4, 5, 6, 7, 8, 9, or 10 minutes of sequestration. In some embodiments, a labile bile acid sequestrant releases a bile acid or is degraded after about 15, 20, 25, 30, 35, 45, 50, or 55 minutes of sequestration. In some embodiments, a labile bile acid sequestrant releases a bile acid or is degraded after about 1, 2, 3, 4, 5, 6, 7, 8, 9, 10, 11, 12, 13, 14, 15, 16, 17, 18, 19, 20, 21, 22, 23, or 24 hours of sequestration. In some embodiments, a labile bile acid sequestrant releases a bile acid or is degraded after 1, 2, or 3 days of sequestration.

In some embodiments, the labile bile acid sequestrant has a low affinity for bile acid. In certain embodiments, the labile bile acid sequestrant has a high affinity for a primary bile acid and a low affinity for a secondary bile acid.

In some embodiments, the labile bile acid sequestrant is a pH dependent bile acid sequestrant. In certain embodiments, the pH dependent bile acid sequestrant has a high affinity for bile acid at a pH of 6 or below and a low affinity for bile acid at a pH above 6. In certain embodiments, the pH dependent bile acid sequestrant has a high affinity for bile acid at a pH of 6.5 or below and a low affinity for bile acid at a pH above 6.5. In certain embodiments, the pH dependent bile acid sequestrant has a high affinity for bile acid at a pH of 7 or below and a low affinity for bile acid at a pH above 7. In certain embodiments, the pH dependent bile acid sequestrant has a high affinity for bile acid at a pH of 7.1 or below and a low affinity for bile acid at a pH above 7.1. In certain embodiments, the pH dependent bile acid sequestrant has a high affinity for bile acid at a pH of 7.2 or below and a low affinity for bile acid at a pH above 7.2. In certain embodiments, the pH dependent bile acid sequestrant has a high affinity for bile acid at a pH of 7.3 or below and a low affinity for bile acid at a pH above 7.3. In certain embodiments, the pH dependent bile acid sequestrant has a high affinity for bile acid at a pH of 7.4 or below and a low affinity for bile acid at a pH above 7.4. In certain embodiments, the pH dependent bile acid sequestrant has a high affinity for bile acid at a pH of 7.5 or below and a low affinity for bile acid at a pH above 7.5. In certain embodiments, the pH dependent bile acid sequestrant has a high affinity for bile acid at a pH of 7.6 or below and a low affinity for bile acid at a pH above 7.6. In certain embodiments, the pH dependent bile acid sequestrant has a high affinity for bile acid at a pH of 7.7 or below and a low affinity for bile acid at a pH above 7.7. In certain embodiments, the pH dependent bile acid sequestrant has a high affinity for bile acid at a pH of 7.8 or below and a low affinity for bile acid at a pH above 7.8. In some embodiments, the pH dependent bile acid sequestrant degrades at a pH above 6. In some embodiments, the pH dependent bile acid sequestrant degrades at a pH above 6.5. In some embodiments, the pH dependent bile acid sequestrant degrades at a pH above 7. In some embodiments, the pH dependent bile acid sequestrant degrades at a pH above 7.1. In some embodiments, the pH dependent bile acid sequestrant degrades at a pH above 7.2. In some embodiments, the pH dependent bile acid sequestrant degrades at a pH above 7.3. In some embodiments, the pH dependent bile acid sequestrant degrades at a pH above 7.4. In some embodiments, the pH dependent bile acid sequestrant degrades at a pH above 7.5. In some embodiments, the pH dependent bile acid sequestrant degrades at a pH above 7.6. In some embodiments, the pH dependent bile acid sequestrant degrades at a pH above 7.7. In some embodiments, the pH dependent bile acid sequestrant degrades at a pH above 7.8. In some embodiments, the pH dependent bile acid sequestrant degrades at a pH above 7.9.

In certain embodiments, the labile bile acid sequestrant is lignin or a modified lignin. In some embodiments, the labile bile acid sequestrant is a polycationic polymer or copolymer. In certain embodiments, the labile bile acid sequestrant is a polymer or copolymer comprising one or more N-alkenyl-N-alkylamine residues; one or more N,N,N-trialkyl-N—(N′-alkenylamino)alkyl-azanium residues; one or more N,N,N-trialkyl-N-alkenyl-azanium residues; one or more alkenyl-amine residues; or a combination thereof. In some embodiments, the bile acid binder is cholestyramine, and various compositions including cholestyramine, which are described, for example, in U.S. Pat. Nos. 3,383,281; 3,308,020; 3,769,399; 3,846,541; 3,974,272; 4,172,120; 4,252,790; 4,340,585; 4,814,354; 4,874,744; 4,895,723; 5,695,749; and 6,066,336, all of which are incorporated herein by reference in their entirety for all purposes. In some embodiments, the bile acid binder is cholestipol or cholesevelam.

### Routes of Administration, Dosage Forms, and Dosing Regimens

In some embodiments, the compositions described herein, and the compositions administered in the methods described herein are formulated to inhibit bile acid reuptake or reduce serum or hepatic bile acid levels. In certain embodiments, the compositions described herein are formulated for rectal or oral administration. In some embodiments, such formulations are administered rectally or orally, respectively. In some embodiments, the compositions described herein are combined with a device for local delivery of the compositions to the rectum and/or colon (sigmoid colon, transverse colon, or ascending colon). In certain embodiments, for rectal administration the composition described herein are formulated as enemas, rectal gels, rectal foams, rectal aerosols, suppositories, jelly suppositories, or retention enemas. In some embodiments, for oral administration the compositions described herein are formulated for oral administration and enteric delivery to the colon.

In certain embodiments, the compositions or methods described herein are non-systemic. In some embodiments, compositions described herein deliver the IBAT inhibitor to the distal ileum, colon, and/or rectum and not systemically (e.g., a substantial portion of the enteroendocrine peptide secretion enhancing agent is not systemically absorbed). In some embodiments, oral compositions described herein deliver the IBAT inhibitor to the distal ileum, colon, and/or rectum and not systemically (e.g., a substantial portion of the enteroendocrine peptide secretion enhancing agent is not systemically absorbed). In some embodiments, rectal compositions described herein deliver the IBAT inhibitor to the distal ileum, colon, and/or rectum and not systemically (e.g., a substantial portion of the enteroendocrine peptide secretion enhancing agent is not systemically absorbed). In certain embodiments, non-systemic compositions described herein deliver less than 90% w/w of the IBAT inhibitor systemically. In certain embodiments, non-systemic compositions described herein deliver less than 80% w/w of the IBAT inhibitor systemically. In certain embodiments, non-systemic compositions described herein deliver less than 70% w/w of the IBAT inhibitor systemically. In certain embodiments, non-systemic compositions described herein deliver less than 60% w/w of the IBAT inhibitor systemically. In certain embodiments, non-systemic compositions described herein deliver less than 50% w/w of the IBAT inhibitor systemically. In certain embodiments, non-systemic compositions described herein deliver less than 40% w/w of the IBAT inhibitor systemically. In certain embodiments, non-systemic compositions described herein deliver less than 30% w/w of the IBAT inhibitor systemically. In certain embodiments, non-systemic compositions described herein deliver less than 25% w/w of the IBAT inhibitor systemically. In certain embodiments, non-systemic compositions described herein deliver less than 20% w/w of the IBAT inhibitor systemically. In certain embodiments, non-systemic compositions described herein deliver less than 15% w/w of the IBAT inhibitor systemically. In certain embodiments, non-systemic compositions described herein deliver less than 10% w/w of the IBAT inhibitor systemically. In certain embodiments, non-systemic compositions described herein deliver less than 5% w/w of the IBAT inhibitor systemically. In some embodiments, systemic absorption is determined in any suitable manner, including the total circulating amount, the amount cleared after administration, or the like.

In certain embodiments, the compositions and/or formulations described herein are administered at least once a day. In certain embodiments, the formulations containing the IBAT inhibitor are administered at least twice a day, while in other embodiments the formulations containing the IBAT inhibitor are administered at least three times a day. In certain embodiments, the formulations containing the IBAT inhibitor are administered up to five times a day. It is to be understood that in certain embodiments, the dosage regimen of composition containing the IBAT inhibitor described herein to is determined by considering various factors such as the patient's age, sex, and diet.

The concentration of the IBAT inhibitor administered in the formulations described herein ranges from about 1 mM to about 1 M. In certain embodiments the concentration of the IBAT inhibitor administered in the formulations described herein ranges from about 1 mM to about 750 mM. In certain embodiments the concentration of the IBAT inhibitor administered in the formulations described herein ranges from about 1 mM to about 500 mM. In certain embodiments the concentration of the IBAT inhibitor administered in the formulations described herein ranges from about 5 mM to about 500 mM. In certain embodiments the concentration of the IBAT inhibitor administered in the formulations described herein ranges from about 10 mM to about 500 mM. In certain embodiments the concentration of the administered in the formulations described herein ranges from about 25 mM to about 500 mM. In certain embodiments the concentration of the IBAT inhibitor administered in the formulations described herein ranges from about 50 mM to about 500 mM. In certain embodiments the concentration of the IBAT inhibitor administered in the formulations described herein ranges from about 100 mM to about 500 mM. In certain embodiments the concentration of the IBAT inhibitor administered in the formulations described herein ranges from about 200 mM to about 500 mM.

In certain embodiments, by targeting the distal gastrointestinal tract (e.g., distal ileum, colon, and/or rectum), compositions and methods described herein provide efficacy (e.g., in reducing microbial growth and/or alleviating symptoms of cholestasis or a cholestatic liver disease) with a reduced dose of enteroendocrine peptide secretion enhancing agent (e.g., as compared to an oral dose that does not target the distal gastrointestinal tract).

### Oral Administration for Colonic Delivery

In certain aspects, the composition or formulation containing one or more compounds described herein is orally administered for local delivery of an MAT inhibitor, or a compound described herein to the colon and/or rectum. Unit dosage forms of such compositions include a pill, tablet or capsules formulated for enteric delivery to colon. In certain embodiments, such pills, tablets or capsule contain the compositions described herein entrapped or embedded in microspheres. In some embodiments, microspheres include, by way of non-limiting example, chitosan microcores HPMC capsules and cellulose acetate butyrate (CAB) microspheres. In certain embodiments, oral dosage forms are prepared using conventional methods known to those in the field of pharmaceutical formulation. For example, in certain embodiments, tablets are manufactured using standard tablet processing procedures and equipment. An exemplary method for forming tablets is by direct compression of a powdered, crystalline or granular composition containing the active agent(s), alone or in combination with one or more carriers, additives, or the like. In alternative embodiments, tablets are prepared using wet-granulation or dry-granulation processes. In some embodiments, tablets are molded rather than compressed, starting with a moist or otherwise tractable material.

In certain embodiments, tablets prepared for oral administration contain various excipients, including, by way of non-limiting example, binders, diluents, lubricants, disintegrants, fillers, stabilizers, surfactants, preservatives, coloring agents, flavoring agents and the like. In some embodiments, binders are used to impart cohesive qualities to a tablet, ensuring that the tablet remains intact after compression. Suitable binder materials include, by way of non-limiting example, stareh (including corn stareh and pregelatinized stareh), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), Veegum, and combinations thereof. In certain embodiments, diluents are utilized to increase the bulk of the tablet so that a practical size tablet is provided. Suitable diluents include, by way of non-limiting example, dicalcium phosphate, calcium sulfate, lactose, cellulose, kaolin, mannitol, sodium chloride, dry stareh, powdered sugar and combinations thereof. In certain embodiments, lubricants are used to facilitate tablet manufacture; examples of suitable lubricants include, by way of non-limiting example, vegetable oils such as peanut oil, cottonseed oil, sesame oil, olive oil, corn oil, and oil of theobroma, glycerin, magnesium stearate, calcium stearate, stearic acid and combinations thereof. In some embodiments, disintegrants are used to facilitate disintegration of the tablet, and include, by way of non-limiting example, starehes, clays, celluloses, algins, gums, crosslinked polymers and combinations thereof. Fillers include, by way of non-limiting example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. In certain embodiments, stabilizers are used to inhibit or retard drug decomposition reactions that include, by way of example, oxidative reactions. In certain embodiments, surfactants are anionic, cationic, amphoteric or nonionic surface active agents.

In some embodiments, IBAT inhibitors, or other compounds described herein are orally administered in association with a carrier suitable for delivery to the distal gastrointestinal tract (e.g., distal ileum, colon, and/or rectum).

In certain embodiments, a composition described herein comprises an IBAT inhibitor, or other compounds described herein in association with a matrix (e.g., a matrix comprising hypermellose) that allows for controlled release of an active agent in the distal part of the ileum and/or the colon. In some embodiments, a composition comprises a polymer that is pH sensitive (e.g., a MMX™ matrix from Cosmo Pharmaceuticals) and allows for controlled release of an active agent in the distal part of the ileum. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO₃H) and swell in basic pH of the intestine (e.g., pH of about 7 to about 8). In some embodiments, a composition suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an enteroendocrine peptide secretion enhancing agent to the distal ileum. In some embodiments, a dosage form comprising an enteroendocrine peptide secretion enhancing agent is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the distal ileum and/or the colon. In some embodiments, bacterially activated systems are suitable for targeted delivery to the distal part of the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pharmaceutical composition described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some embodiments, using standard coating procedures, such as those described in Remington's Pharmaceutical Sciences, 20th Edition (2000), a film coating is provided around the formulation of the compound of Formula I. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

In further embodiments, a tablet or capsule comprising an IBAT inhibitor or other compounds described herein is film-coated for delivery to targeted sites within the gastrointestinal tract. Examples of enteric film coats include and are not limited to hydroxypropylmethylcellulose, polyvinyl pyrrolidone, hydroxypropyl cellulose, polyethylene glycol 3350, 4500, 8000, methyl cellulose, pseudoethylcellulose, amylopectin and the like.

### Pediatric Dosage Formulations and Compositions

Provided herein, in certain embodiments, is a pediatric dosage formulation or composition comprising a therapeutically effective amount of any compound described herein. In certain instances, the pharmaceutical composition comprises an IBAT inhibitor (e.g., any IBAT inhibitor described herein).

In certain embodiments, suitable dosage forms for the pediatric dosage formulation or composition include, by way of non-limiting example, aqueous or non-aqueous oral dispersions, liquids, gels, syrups, elixirs, slurries, suspensions, solutions, controlled release formulations, fast melt formulations, effervescent formulations, lyophilized formulations, chewable tablets, gummy candy, orally disintegrating tablets, powders for reconstitution as suspension or solution, sprinkle oral powder or granules, dragees, delayed release formulations, extended release formulations, pulsatile release formulations, multiparticulate formulations, and mixed immediate release and controlled release formulations. In some embodiments, provided herein is a pharmaceutical composition wherein the pediatric dosage form is selected from a solution, syrup, suspension, elixir, powder for reconstitution as suspension or solution, dispersible/effervescent tablet, chewable tablet, gummy candy, lollipop, freezer pops, troches, oral thin strips, orally disintegrating tablet, orally disintegrating strip, sachet, and sprinkle oral powder or granules.

In another aspect, provide herein is a pharmaceutical composition wherein at least one excipient is a flavoring agent or a sweetener. In some embodiments, provided herein is a coating. In some embodiments, provided herein is a taste-masking technology selected from coating of drug particles with a taste-neutral polymer by spray-drying, wet granulation, fluidized bed, and microencapsulation; coating with molten waxes of a mixture of molten waxes and other pharmaceutical adjuvants; entrapment of drug particles by complexation, flocculation or coagulation of an aqueous polymeric dispersion; adsorption of drug particles on resin and inorganic supports; and solid dispersion wherein a drug and one or more taste neutral compounds are melted and cooled, or co-precipitated by a solvent evaporation. In some embodiments, provided herein is a delayed or sustained release formulation comprising drug particles or granules in a rate controlling polymer or matrix.

Suitable sweeteners include sucrose, glucose, fructose or intense sweeteners, i.e. agents with a high sweetening power when compared to sucrose (e.g. at least 10 times sweeter than sucrose). Suitable intense sweeteners comprise aspartame, saccharin, sodium or potassium or calcium saccharin, acesulfame potassium, sucralose, alitame, xylitol, cyclamate, neomate, neohesperidine dihydrochalcone or mixtures thereof, thaumatin, palatinit, stevioside, rebaudioside, Magnasweet®. The total concentration of the sweeteners may range from effectively zero to about 300 mg/ml based on the liquid composition upon reconstitution.

In order to increase the palatability of the liquid composition upon reconstitution with an aqueous medium, one or more taste-making agents may be added to the composition in order to mask the taste of the IBAT inhibitor. A taste-masking agent can be a sweetener, a flavoring agent or a combination thereof. The taste-masking agents typically provide up to about 0.1% or 5% by weight of the total pharmaceutical composition. In a preferred embodiment of the present invention, the composition contains both sweetener(s) and flavor(s).

A flavoring agent herein is a substance capable of enhancing taste or aroma of a composition. Suitable natural or synthetic flavoring agents can be selected from standard reference books, for example Fenaroli's Handbook of Flavor Ingredients, 3rd edition (1995). Non-limiting examples of flavoring agents and/or sweeteners useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. Flavoring agents can be used singly or in combinations of two or more. In some embodiments, the aqueous liquid dispersion comprises a sweetening agent or flavoring agent in a concentration ranging from about 0.001% to about 5.0% the volume of the aqueous dispersion. In one embodiment, the aqueous liquid dispersion comprises a sweetening agent or flavoring agent in a concentration ranging from about 0.001% to about 1.0% the volume of the aqueous dispersion. In another embodiment, the aqueous liquid dispersion comprises a sweetening agent or flavoring agent in a concentration ranging from about 0.005% to about 0.5% the volume of the aqueous dispersion. In yet another embodiment, the aqueous liquid dispersion comprises a sweetening agent or flavoring agent in a concentration ranging from about 0.01% to about 1.0% the volume of the aqueous dispersion. In yet another embodiment, the aqueous liquid dispersion comprises a sweetening agent or flavoring agent in a concentration ranging from about 0.01% to about 0.5% the volume of the aqueous dispersion.

In certain embodiments, a pediatric pharmaceutical composition described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” for pediatric pharmaceutical compositions includes, in some embodiments, a pharmaceutically acceptable excipient and is selected on the basis of compatibility with compounds described herein, such as, compounds of any of Formula I-VI, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), all of which references are incorporated herein by reference in their entirety for all purposes.

Moreover, in certain embodiments, the pediatric pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In certain aspects, the pediatric composition or formulation containing one or more compounds described herein is orally administered for local delivery of an IBAT inhibitor, or a compound described herein to the colon and/or rectum. Unit dosage forms of such compositions include a pill, tablet or capsules formulated for enteric delivery to colon.

In some embodiments, IBAT inhibitors, or other compounds described herein are orally administered in association with a carrier suitable for delivery to the distal gastrointestinal tract (e.g., distal ileum, colon, and/or rectum).

In certain embodiments, a pediatric composition described herein comprises an IBAT inhibitor, or other compounds described herein in association with a matrix (e.g., a matrix comprising hypermellose) that allows for controlled release of an active agent in the distal part of the ileum and/or the colon. In some embodiments, a composition comprises a polymer that is pH sensitive (e.g., a MMX™ matrix from Cosmo Pharmaceuticals) and allows for controlled release of an active agent in the distal part of the ileum. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO₃H) and swell in basic pH of the intestine (e.g., pH of about 7 to about 8). In some embodiments, a composition suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an enteroendocrine peptide secretion enhancing agent to the distal ileum. In some embodiments, a dosage form comprising an enteroendocrine peptide secretion enhancing agent is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the distal ileum and/or the colon. In some embodiments, bacterially activated systems are suitable for targeted delivery to the distal part of the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pediatric pharmaceutical composition described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the compound of Formula I. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

### Liquid Dosage Forms

The pharmaceutical liquid dosage forms of the invention may be prepared according to techniques well-known in the art of pharmacy.

A solution refers to a liquid pharmaceutical formulation wherein the active ingredient is dissolved in the liquid. Pharmaceutical solutions of the invention include syrups and elixirs. A suspension refers to a liquid pharmaceutical formulation wherein the active ingredient is in a precipitate in the liquid.

In a liquid dosage form, it is desirable to have a particular pH and/or to be maintained within a specific pH range. In order to control the pH, a suitable buffer system can be used. In addition, the buffer system should have sufficient capacity to maintain the desired pH range. Examples of the buffer system useful in the present invention include but are not limited to, citrate buffers, phosphate buffers, or any other suitable buffer known in the art. Preferably the buffer system include sodium citrate, potassium citrate, sodium bicarbonate, potassium bicarbonate, sodium dihydrogen phosphate and potassium dihydrogen phosphate, etc. The concentration of the buffer system in the final suspension varies according to factors such as the strength of the buffer system and the pH/pH ranges required for the liquid dosage form. In one embodiment, the concentration is within the range of 0.005 to 0.5 w/v % in the final liquid dosage form.

The pharmaceutical composition comprising the liquid dosage form of the present invention can also include a suspending/stabilizing agent to prevent settling of the active material. Over time the settling could lead to caking of the active to the inside walls of the product pack, leading to difficulties with redispersion and accurate dispensing. Suitable stabilizing agents include but are not limited to, the polysaccharide stabilizers such as xanthan, guar and tragacanth gums as well as the cellulose derivatives HPMC (hydroxypropyl methylcellulose), methyl cellulose and Avicel RC-591 (microcrystalline cellulose/sodium carboxymethyl cellulose). In another embodiment, polyvinylpyrrolidone (PVP) can also be used as a stabilizing agent.

In addition to the aforementioned components, the IBAT inhibitor oral suspension form can also optionally contain other excipients commonly found in pharmaceutical compositions such as alternative solvents, taste-masking agents, antioxidants, fillers, acidifiers, enzyme inhibitors and other components as described in Handbook of Pharmaceutical Excipients, Rowe et al., Eds., 4^{th} Edition, Pharmaceutical Press (2003), which is hereby incorporated by reference in its entirety for all purposes.

Addition of an alternative solvent may help increase solubility of an active ingredient in the liquid dosage form, and consequently the absorption and bioavailability inside the body of a subject. Preferably the alternative solvents include methanol, ethanol or propylene glycol and the like.

In another aspect, the present invention provides a process for preparing the liquid dosage form. The process comprises steps of bringing IBAT inhibitor or its pharmaceutically acceptable salts thereof into mixture with the components including glycerol or syrup or the mixture thereof, a preservative, a buffer system and a suspending/stabilizing agent, etc., in a liquid medium. In general, the liquid dosage form is prepared by uniformly and intimately mixing these various components in the liquid medium. For example, the components such as glycerol or syrup or the mixture thereof, a preservative, a buffer system and a suspending/stabilizing agent, etc., can be dissolved in water to form the aqueous solution, then the active ingredient can be then dispersed in the aqueous solution to form a suspension.

In some embodiments, the liquid dosage form provided herein can be in a volume of between about 0.1 ml to about 50 ml. In some embodiments, the liquid dosage form provided herein can be in a volume of between about 0.2 ml to about 40 ml. In some embodiments, the liquid dosage form provided herein can be in a volume of between about 0.5 ml to about 30 ml. In some embodiments, the liquid dosage form provided herein can be in a volume of between about 1 ml to about 20 ml. In some embodiments, the liquid dosage form provided herein can be in a volume of between about 0.1 ml to about 20 ml. In some embodiments, the liquid dosage form provided herein can be in a volume of about 0.1 ml to about 20 ml. In some embodiments, the IBAT inhibitor can be in an amount ranging from about 0.001% to about 90% of the total volume. In some embodiments, the IBAT inhibitor can be in an amount ranging from about 0.01% to about 80% of the total volume. In some embodiments, the IBAT inhibitor can be in an amount ranging from about 0.1% to about 70% of the total volume. In some embodiments, the IBAT inhibitor can be in an amount ranging from about 1% to about 60% of the total volume. In some embodiments, the IBAT inhibitor can be in an amount ranging from about 1% to about 50% of the total volume. In some embodiments, the IBAT inhibitor can be in an amount ranging from about 1% to about 40% of the total volume. In some embodiments, the IBAT inhibitor can be in an amount ranging from about 1% to about 30% of the total volume. In some embodiments, the IBAT inhibitor can be in an amount ranging from about 1% to about 20% of the total volume. In some embodiments, the IBAT inhibitor can be in an amount ranging from about 1% to about 10% of the total volume. In some embodiments, the IBAT inhibitor can be in an amount ranging from about 5% to about 70% of the total volume. In some embodiments, the IBAT inhibitor can be in an amount ranging from about 5% to about 60% of the total volume. In some embodiments, the IBAT inhibitor can be in an amount ranging from about 5% to about 50% of the total volume. In some embodiments, the IBAT inhibitor can be in an amount ranging from about 5% to about 40% of the total volume. In some embodiments, the IBAT inhibitor can be in an amount ranging from about 5% to about 30% of the total volume. In some embodiments, the IBAT inhibitor can be in an amount ranging from about 5% to about 20% of the total volume. In some embodiments, the IBAT inhibitor can be in an amount ranging from about 5% to about 10% of the total volume. In some embodiments, the IBAT inhibitor can be in an amount ranging from about 10% to about 50% of the total volume. In some embodiments, the IBAT inhibitor can be in an amount ranging from about 10% to about 40% of the total volume. In some embodiments, the IBAT inhibitor can be in an amount ranging from about 10% to about 30% of the total volume. In some embodiments, the IBAT inhibitor can be in an amount ranging from about 10% to about 20% of the total volume. In one embodiment, the resulted liquid dosage form can be in a liquid volume of 0. ml to 30 ml, preferably 0.1 ml to 20 ml, and the active ingredient can be in an amount ranging from about 0.001 mg/ml to about 16 mg/ml, or from about 0.025 mg/ml to about 8 mg/ml, or from about 0.1 mg/ml to about 4 mg/ml, or about 0.25 mg/ml, or about 0.5 mg/ml, or about 1 mg/ml, or about 2 mg/ml, or about 4 mg/ml, or about 5 mg/ml, or about 8 mg/ml, or about 9 mg/ml, or about 10 mg/ml, or about 12 mg/ml, or about 14 mg/ml or about 16 mg/ml.

### Bile Acid Sequestrant

In certain embodiments, an oral formulation for use in any method described herein is, e.g., an IBAT inhibitor in association with a labile bile acid sequestrant. A labile bile acid sequestrant is a bile acid sequestrant with a labile affinity for bile acids. In certain embodiments, a bile acid sequestrant described herein is an agent that sequesters (e.g., absorbs or is charged with) bile acid, and/or the salts thereof.

In specific embodiments, the labile bile acid sequestrant is an agent that sequesters (e.g., absorbs or is charged with) bile acid, and/or the salts thereof, and releases at least a portion of the absorbed or charged bile acid, and/or salts thereof in the distal gastrointestinal tract (e.g., the colon, ascending colon, sigmoid colon, distal colon, rectum, or any combination thereof). In certain embodiments, the labile bile acid sequestrant is an enzyme dependent bile acid sequestrant. In specific embodiments, the enzyme is a bacterial enzyme. In some embodiments, the enzyme is a bacterial enzyme found in high concentration in human colon or rectum relative to the concentration found in the small intestine. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like. In some embodiments, the labile bile acid sequestrant is a time dependent bile acid sequestrant (i.e., the bile acid sequesters the bile acid and/or salts thereof and after a time releases at least a portion of the bile acid and/or salts thereof). In some embodiments, a time dependent bile acid sequestrant is an agent that degrades in an aqueous environment over time. In certain embodiments, a labile bile acid sequestrant described herein is a bile acid sequestrant that has a low affinity for bile acid and/or salts thereof, thereby allowing the bile acid sequestrant to continue to sequester bile acid and/or salts thereof in an environ where the bile acids/salts and/or salts thereof are present in high concentration and release them in an environ wherein bile acids/salts and/or salts thereof are present in a lower relative concentration. In some embodiments, the labile bile acid sequestrant has a high affinity for a primary bile acid and a low affinity for a secondary bile acid, allowing the bile acid sequestrant to sequester a primary bile acid or salt thereof and subsequently release a secondary bile acid or salt thereof as the primary bile acid or salt thereof is converted (e.g., metabolized) to the secondary bile acid or salt thereof. In some embodiments, the labile bile acid sequestrant is a pH dependent bile acid sequestrant. In some embodiments, the pH dependent bile acid sequestrant has a high affinity for bile acid at a pH of 6 or below and a low affinity for bile acid at a pH above 6. In certain embodiments, the pH dependent bile acid sequestrant degrades at a pH above 6.

In some embodiments, labile bile acid sequestrants described herein include any compound, e.g., a macro-structured compound, that can sequester bile acids/salts and/or salts thereof through any suitable mechanism. For example, in certain embodiments, bile acid sequestrants sequester bile acids/salts and/or salts thereof through ionic interactions, polar interactions, static interactions, hydrophobic interactions, lipophilic interactions, hydrophilic interactions, steric interactions, or the like. In certain embodiments, macrostructured compounds sequester bile acids/salts and/or sequestrants by trapping the bile acids/salts and/or salts thereof in pockets of the macrostructured compounds and, optionally, other interactions, such as those described above. In some embodiments, bile acid sequestrants (e.g., labile bile acid sequestrants) include, by way of non-limiting example, lignin, modified lignin, polymers, polycationic polymers and copolymers, polymers and/or copolymers comprising anyone one or more of N-alkenyl-N-alkylaminc residues; one or more N,N,N-trialkyl-N—(N′-alkenyl amino)alkyl-azanium residues; one or more N,N,N-trialkyl-N-alkenyl-azanium residues; one or more alkenyl-amine residues; or a combination thereof, or any combination thereof.

Covalent Linkage of the Drug with a Carrier

In some embodiments, strategies used for colon targeted delivery include, by way of non-limiting example, covalent linkage of the IBAT inhibitor or other compounds described herein to a carrier, coating the dosage form with a pH-sensitive polymer for delivery upon reaching the pH environment of the colon, using redox sensitive polymers, using a time released formulation, utilizing coatings that are specifically degraded by colonic bacteria, using bioadhesive system and using osmotically controlled drug delivery systems.

In certain embodiments of such oral administration of a composition containing an IBAT inhibitor or other compounds described herein involves covalent linking to a carrier wherein upon oral administration the linked moiety remains intact in the stomach and small intestine. Upon entering the colon, the covalent linkage is broken by the change in pH, enzymes, and/or degradation by intestinal microflora. In certain embodiments, the covalent linkage between the IBAT inhibitor and the carrier includes, by way of non-limiting example, azo linkage, glycoside conjugates, glucuronide conjugates, cyclodextrin conjugates, dextran conjugates, and amino-acid conjugates (high hydrophilicity and long chain length of the carrier amino acid).

Coating with Polymers: pH-Sensitive Polymers

In some embodiments, the oral dosage forms described herein are coated with an enteric coating to facilitate the delivery of an IBAT inhibitor or other compounds described herein to the colon and/or rectum. In certain embodiments, an enteric coating is one that remains intact in the low pH environment of the stomach, but readily dissolved when the optimum dissolution pH of the particular coating is reached which depends upon the chemical composition of the enteric coating. The thickness of the coating will depend upon the solubility characteristics of the coating material. In certain embodiments, the coating thicknesses used in such formulations described herein range from about 25 μm to about 200 μm.

In certain embodiments, the compositions or formulations described herein are coated such that an MAT inhibitor or other compounds described herein of the composition or formulation is delivered to the colon and/or rectum without absorbing at the upper part of the intestine. In a specific embodiment, specific delivery to the colon and/or rectum is achieved by coating of the dosage form with polymers that degrade only in the pH environment of the colon. In alternative embodiments, the composition is coated with an enteric coat that dissolves in the pH of the intestines and an outer layer matrix that slowly erodes in the intestine. In some of such embodiments, the matrix slowly erodes until only a core composition comprising an enteroendocrine peptide secretion enhancing agent (and, in some embodiments, an absorption inhibitor of the agent) is left and the core is delivered to the colon and/or rectum.

In certain embodiments, pH-dependent systems exploit the progressively increasing pH along the human gastrointestinal tract (GIT) from the stomach (pH 1-2 which increases to 4 during digestion), small intestine (pH 6-7) at the site of digestion and it to 7-8 in the distal ileum. In certain embodiments, dosage forms for oral administration of the compositions described herein are coated with pH-sensitive polymer(s) to provide delayed release and protect the enteroendocrine peptide secretion enhancing agents from gastric fluid. In certain embodiments, such polymers are be able to withstand the lower pH values of the stomach and of the proximal part of the small intestine but disintegrate at the neutral or slightly alkaline pH of the terminal ileum and/or ileocecal junction. Thus, in certain embodiments, provided herein is an oral dosage form comprising a coating, the coating comprising a pH-sensitive polymer. In some embodiments, the polymers used for colon and/or rectum targeting include, by way of non-limiting example, methacrylic acid copolymers, methacrylic acid and methyl methacrylate copolymers, Eudragit L100, Eudragit S100, Eudragit L-30D, Eudragit FS-30D, Eudragit L100-55, polyvinylacetate phthalate, hyrdoxypropyl ethyl cellulose phthalate, hyrdoxypropyl methyl cellulose phthalate 50, hyrdoxypropyl methyl cellulose phthalate 55, cellulose acetate trimelliate, cellulose acetate phthalate and combinations thereof.

In certain embodiments, oral dosage forms suitable for delivery to the colon and/or rectum comprise a coating that has a biodegradable and/or bacteria degradable polymer or polymers that are degraded by the microflora (bacteria) in the colon. In such biodegradable systems suitable polymers include, by way of non-limiting example, azo polymers, linear-type-segmented polyurethanes containing azo groups, polygalactomannans, pectin, glutaraldehyde crosslinked dextran, polysaccharides, amylose, guar gum, pectin, chitosan, inulin, cyclodextrins, chondroitin sulphate, dextrans, locust bean gum, chondroitin sulphate, chitosan, poly (-caprolactone), polylactic acid and poly(lactic-co-glycolic acid).

In certain embodiments of such oral administration of compositions containing one or more MAT inhibitors or other compounds described herein, the compositions are delivered to the colon without absorbing at the upper part of the intestine by coating of the dosage forms with redox sensitive polymers that are degraded by the microflora (bacteria) in the colon. In such biodegradable systems such polymers include, by way of non-limiting example, redox-sensitive polymers containing an azo and/or a disulfide linkage in the backbone.

In some embodiments, compositions formulated for delivery to the colon and/or rectum are formulated for time-release. In some embodiments, time release formulations resist the acidic environment of the stomach, thereby delaying the release of the enteroendocrine peptide secretion enhancing agents until the dosage form enters the colon and/or rectum.

In certain embodiments the time released formulations described herein comprise a capsule (comprising an enteroendocrine peptide secretion enhancing agent and an optional absorption inhibitor) with hydrogel plug. In certain embodiments, the capsule and hydrogel plug are covered by a water-soluble cap and the whole unit is coated with an enteric polymer. When the capsule enters the small intestine the enteric coating dissolves and the hydrogels plug swells and dislodges from the capsule after a period of time and the composition is released from the capsule. The amount of hydrogel is used to adjust the period of time to the release the contents.

In some embodiments, provided herein is an oral dosage form comprising a multi-layered coat, wherein the coat comprises different layers of polymers having different pH-sensitivities. As the coated dosage form moves along GIT the different layers dissolve depending on the pH encountered. Polymers used in such formulations include, by way of non-limiting example, polymethacrylates with appropriate pH dissolution characteristics, Eudragit® RL and Eudragit®RS (inner layer), and Eudragit® FS (outer layer). In other embodiments the dosage form is an enteric coated tablets having an outer shell of hydroxypropylcellulose or hydroxypropylmethylcellulose acetate succinate (HPMCAS).

In some embodiments, provided herein is an oral dosage form that comprises coat with cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and combinations thereof.

### Combination Therapy

In some embodiments, the methods provided herein comprise administering a compound (e.g., an IBAT inhibitor) or composition described herein in combination with one or more additional agents. In some embodiments, the present invention also provides a composition comprising a compound (e.g., an IBAT inhibitor) with one or more additional agents.

### Fat Soluble Vitamins

In some embodiments, the methods provided herein further comprise administering one or more vitamins. In some embodiments, the vitamin is vitamin A, B1, B2, B3, B5, B6, B7, B9, B12, C, D, E, K, folic acid, pantothenic acid, niacin, riboflavin, thiamine, retinol, beta carotene, pyridoxine, ascorbic acid, cholecalciferol, cyanocobalamin, tocopherols, phylloquinone, menaquinone.

In some embodiments, the vitamin is a fat soluble vitamin such as vitamin A, D, E, K, retinol, beta carotene, cholecalciferol, tocopherols, phylloquinone. In a preferred embodiment, the fat soluble vitamin is tocopherol polyethylene glycol succinate (TPGS).

### IBAT Inhibitors and PPAR Agonists

In various embodiments, the present invention provides methods of use of combinations of IBAT inhibitors with PPAR (peroxisome proliferator-activated receptor) agonists. In various embodiments, the PPAR agonist is a fibrate drug. In some embodiments, the fibrate drug is clofibrate, gemfibrozil, ciprofibrate, benzafibrate, fenofibrate, or various combinations thereof. In various embodiments, the PPAR agonist is aleglitazar, muraglitazar, tesaglitazar, saroglitazar, GW501516, GW-9662, a thiazolidinedione (TZD), a NSAID (e.g., IBUPROFEN), an indole, or various combinations thereof.

### IBAT Inhibitors and FXR Drugs

In various embodiments, the present invention provides methods of use of combinations of IBAT inhibitors with farnesoid X receptor (FXR) targeting drugs. In various embodiments, the FXR targeting drug is avermectin B1a, bepridil, fluticasone propionate, GW4064, gliquidone, nicardipine, triclosan, CDCA, ivermectin, chlorotrianisene, tribenoside, mometasone furoate, miconazole, amiodarone, butoconazolee, bromocryptine mesylate, pizotifen malate, or various combinations thereof.

### Partial External Biliary Diversion (PEBD)

In some embodiments, the methods provided herein further comprise using partial external biliary diversion as a treatment for patients who have not yet developed cirrhosis. This treatment helps reduce the circulation of bile acids/salts in the liver in order to reduce complications and prevent the need for early transplantation in many patients.

This surgical technique involves isolating a segment of intestine 10 cm long for use as a biliary conduit (a channel for the passage of bile) from the rest of the intestine. One end of the conduit is attached to the gallbladder and the other end is brought out to the skin to form a stoma (a surgically constructed opening to permit the passage of waste). Partial external biliary diversion may be used for patients who are unresponsive to all medical therapy, especially older, larger patients. This procedure may not be of help to young patients such as infants. Partial external biliary diversion may decrease the intensity of the itching and abnormally low levels of cholesterol in the blood.

### IBAT Inhibitor and Ursodiol

In some embodiments, an IBAT inhibitor is administered in combination with ursodiol or ursodeoxycholic acid, chenodeoxycholic acid, cholic acid, taurocholic acid, ursocholic acid, glycocholic acid, glycodeoxycholic acid, taurodeoxycholic acid, taurocholate, glycochenodeoxycholic acid, tauroursodeoxycholic acid. In some instances, an increase in the concentration of bile acids/salts in the distal intestine induces intestinal regeneration, attenuating intestinal injury, reducing bacterial translocation, inhibiting the release of free radical oxygen, inhibiting production of proinflammatory cytokines, or any combination thereof or any combination thereof.

In certain embodiments, the patient is administered ursodiol at a daily dose of about or of at least about 5 mg, 10 mg, 15 mg, 20 mg, 25 mg, 30 mg, 35 mg, 36 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,250 mg, 1,500 mg, 1,750 mg, 2,000 mg, 2,250 mg, 2,500 mg, 2,750 mg, or 3,000 mg. In certain embodiments, the patient is administered ursodiol at a daily dose of about or of no more than about 10 mg, 15 mg, 20 mg, 25 mg, 30 mg, 35 mg, 36 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,250 mg, 1,500 mg, 1,750 mg, 2,000 mg, 2,250 mg, 2,500 mg, 2,750 mg, 3,000 mg, or 3,500 mg. In various embodiments, the patient is administered ursodiol at a daily dose of about or of at least about 3 mg to about 300 mg, about 30 mg to about 250 mg, from about 36 mg to about 200 mg, from about 10 mg to about 3000 mg, from about 1000 mg to about 2000 mg, or from about 1500 to about 1900 mg.

In various embodiments the ursodiol is administered as a tablet. In various embodiments, the ursodiol is administered as a suspension. In various embodiments, the concentration of ursodiol in the suspension is from about 10 mg/mL to about 200 mg/mL, from about 50 mg/mL to about 150 mg/mL, from about 10 mg/mL to about 500 mg/mL, or from about 40 mg/mL to about 60 mg/mL. In various embodiments, the concentration of ursodiol in suspension is about or is at least about 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, or 80 mg/mL. In various embodiments, the concentration of ursodiol in suspension is no more than about 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, or 85 mg/mL.

An IBAT inhibitor and a second active ingredient are used such that the combination is present in a therapeutically effective amount. That therapeutically effective amount arises from the use of a combination of an IBAT inhibitor and the other active ingredient (e.g., ursodiol) wherein each is used in a therapeutically effective amount, or by virtue of additive or synergistic effects arising from the combined use, each can also be used in a subclinical therapeutically effective amount, i.e., an amount that, if used alone, provides for reduced effectiveness for the therapeutic purposes noted herein, provided that the combined use is therapeutically effective. In some embodiments, the use of a combination of an IBAT inhibitor and any other active ingredient as described herein encompasses combinations where the IBAT inhibitor or the other active ingredient is present in a therapeutically effective amount, and the other is present in a subclinical therapeutically effective amount, provided that the combined use is therapeutically effective owing to their additive or synergistic effects. As used herein, the term “additive effect” describes the combined effect of two (or more) pharmaceutically active agents that is equal to the sum of the effect of each agent given alone. A syngergistic effect is one in which the combined effect of two (or more) pharmaceutically active agents is greater than the sum of the effect of each agent given alone. Any suitable combination of an IBAT inhibitor with one or more of the aforementioned other active ingredients and optionally with one or more other pharmacologically active substances is contemplated as being within the scope of the methods described herein.

In some embodiments, the particular choice of compounds depends upon the diagnosis of the attending physicians and their judgment of the condition of the individual and the appropriate treatment protocol. The compounds are optionally administered concurrently (e.g., simultaneously, essentially simultaneously or within the same treatment protocol) or sequentially, depending upon the nature of the disease, disorder, or condition, the condition of the individual, and the actual choice of compounds used. In certain instances, the determination of the order of administration, and the number of repetitions of administration of each therapeutic agent during a treatment protocol, is based on an evaluation of the disease being treated and the condition of the individual.

In some embodiments, therapeutically-effective dosages vary when the drugs are used in treatment combinations. Methods for experimentally determining therapeutically-effective dosages of drugs and other agents for use in combination treatment regimens are described in the literature.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a therapeutic compound described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing; e.g, from more than zero weeks to less than four weeks. In addition, the combination methods, compositions and formulations are not to be limited to the use of only two agents; the use of multiple therapeutic combinations are also envisioned (including two or more compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy described herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In certain embodiments, provided herein are combination therapies. In certain embodiments, the compositions described herein comprise an additional therapeutic agent. In some embodiments, the methods described herein comprise administration of a second dosage form comprising an additional therapeutic agent. In certain embodiments, combination therapies the compositions described herein are administered as part of a regimen. Therefore, additional therapeutic agents and/or additional pharmaceutical dosage form can be applied to a patient either directly or indirectly, and concomitantly or sequentially, with the compositions and formulations described herein.

### Kits

In another aspect, provided herein are kits containing a device for oral administration and a pharmaceutical composition as described herein. In certain embodiments the kits include prefilled sachet or bottle for oral administration. In certain embodiments the kits include prefilled syringes for administration of oral enemas.

Release in Distal Ileum and/or Colon

In certain embodiments, a dosage form comprises a matrix (e.g., a matrix comprising hypermellose) that allows for controlled release of an active agent in the distal jejunum, proximal ileum, distal ileum and/or the colon. In some embodiments, a dosage form comprises a polymer that is pH sensitive (e.g., a MMX™ matrix from Cosmo Pharmaceuticals) and allows for controlled release of an active agent in the ileum and/or the colon. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO₃H) and swell in basic pH of the intestine (e.g., pH of about 7 to about 8). In some embodiments, a dosage form suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an IBAT inhibitor to the distal ileum. In some embodiments, a dosage form comprising an IBAT inhibitor is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the ileum and/or the colon. In some embodiments, bacterially activated systems are suitable for targeted delivery to the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the IBAT inhibitor. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

An IBAT inhibitor may be used in the preparation of medicaments for the prophylactic and/or therapeutic treatment of cholestasis or a cholestatic liver disease. A method for treating any of the diseases or conditions described herein in an individual in need of such treatment, may involve administration of pharmaceutical compositions containing at least one IBAT inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

### EXAMPLES

The following examples are provided to further describe some of the embodiments disclosed herein. The examples are intended to illustrate, not to limit, the disclosed embodiments.

### Example 1. A 6-Year Event-Free Survival Analysis in Alagille Syndrome of the GALA Clinical Research Database and Maralixibat Treated Patients

Real-world evidence (RWE) analytics continue to advance natural history comparisons in rare diseases. GALA is the largest global clinical research database for Alagille syndrome (ALGS). Maralixibat (MRX) is an ileal bile acid transporter inhibitor being studied in ALGS. A pre-specified analysis plan applied novel analytical techniques to compare RWE to a MRX cohort with the aim to compare event-free survival (EFS) in patients with ALGS.

GALA contains retrospective data for clinical parameters, biochemistries and outcomes. The MRX database comprises of 84 ALGS patients with up to 6 years of data. EFS was defined as the time to first event of hepatic decompensation (variceal bleeding, ascites requiring therapy), surgical biliary diversion, liver transplantation (LT), or death. GALA was filtered to align key MRX eligibility criteria. The index time was determined via maximum likelihood estimation. Balance among baseline (BL) variables was assessed. Selection of patients and index time was blinded to clinical outcomes. Sensitivity and subgroup analyses, and adjustments for covariates were applied. Missing outcomes data were censored at last contact.

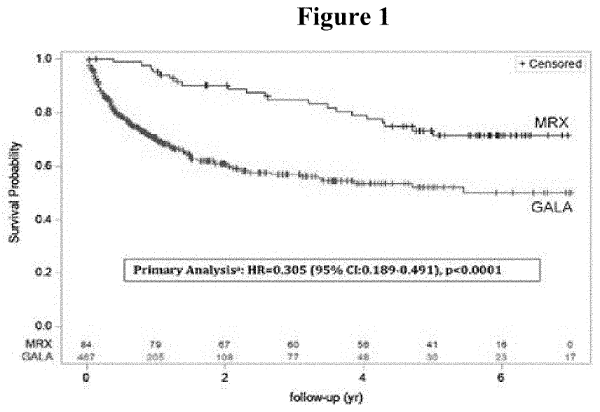

Of 1,438 patients in GALA 469 were eligible. Age, total bilirubin (TB), gamma-glutamyltransferase (GGT), and alanine aminotransferase (ALT) were well balanced between groups and no statistical differences were observed for age, mutation, region, TB, GGT, and ALT. Median BL serum bile acids (sBA) was significantly higher in the MRX cohort (p=0.003); 85% sBA data not available in GALA. EFS rates in the MRX cohort were significantly better than those reported in the GALA control, crude 6-year EFS: 73% and 50%, respectively (FIG. 1), and adjusted for age, sex, TB, ALT: HR=0.305; 95% CI:0.189-0.491; p<0.0001. Varied index times, weighted inverse probability of treatment weights, average treatment effect in the treated, LT and death only, regions, sBA subgroup, pruning events to 12 months were consistent with the primary result. Limitations included no standardized measure of pruritus and limited sBA data in GALA, and inherent bias for patients who enter a clinical trial.

The present 6-year analysis suggested the potential for improved EFS with MRX in patients with ALGS. The present RWE analysis provided a potential method to evaluate outcomes in long-term intervention studies where placebo comparisons are not feasible. Limitations will always be present given lack of prospective conduct and inherent biases, though sensitivity analyses can help mitigate and aid interpretation.

### Example 2. Response to Treatment with Maralixibat in Alagille Syndrome is Associated with Improved Health-Related Quality of Life

Alagille syndrome (ALGS) is associated with high disease burden and diminished health-related quality of life (HRQoL) due to pruritus, failure to thrive and xanthomas. There are no approved drug therapies for ALGS. Maralixibat (MRX), an oral minimally-absorbed inhibitor of the ilealbile acid transporter, has been evaluated for the treatment of cholestatic pruritus in children with ALGS. The impact of MRX treatment response on changes in HRQoL among patients with ALGS was assessed.

A retrospective analysis was performed of 48-week data from the Phase 2 ICONIC trial, a study with a 4-week double-blind, placebo-controlled, randomized drug withdrawal period in patients with ALGS. Patients had moderate to severe pruritus as measured using a validated caregiver-reported Itch Reported Outcome (ItchRO) severity assessment tool (0=none to 4=very severe). Clinically meaningful pruritus response was defined as a ≥1 point reduction in the ItchRO, from baseline to week 48. HRQoL was assessed using the Pediatric Quality of Life Inventory Generic Core (PedsQL; 0-100 scale, 100=best quality of life), Family Impact (FI), and Multidimensional Fatigue (MF) scale scores, and were collected via caregiver. The minimal clinically important difference (MCID) for the HRQoL assessments is 5 points. A subset of individual items from the HRQoL scales were also independently selected for assessment with treatment response. HRQoL scores and selected items were assessed at baseline and week 48 and stratified by treatment response status. P-values were calculated using t-tests or ANOVAs. Multivariate linear regression models were used to assess the relationship between the mean change from baseline in HRQoL score (dependent variable) and an indicator for treatment response (independent variable), adjusting for baseline HRQoL. Adjustment for additional baseline covariates was also explored for the following variables: age, gender, bilirubin, rifampicin use, height z-score, weight z-score, ItchRO, and serum bile acid.

A total of 27 patients with ALGS were included with a mean±SD baseline age of 5.7±4.3 years, ItchRO score of 2.90±0.56, PedsQL score of 59.40±16.97, FI score of 55.14±18.61, MF score of 50.95±23.08, and 67% were male. Baseline patient characteristics, including HRQoL and pruritus scores, were similar among responders and non-responders (Table 1). At week 48, 20 (74%) patients had achieved ItchRO treatment response. Responders had greater mean HRQoL scores than non-responders (Table 2). In addition, responders experienced a greater mean increase in HRQoL from baseline to week 48 compared with non-responders (Table 2).

TABLE 1
Baseline demographic and clinical characteristics
in maralixibat responders and non-responders.

| | ItchRO treatment response at week 48 | | |
|---|---|---|---|
| | | Non- | p- |
| | Responders(N = 20) | responders(N = 7) | value |
| Age (years) | 6.55 ± 4.17 | 3.29 ± 3.99 | 0.08 |
| Male, n (%) | 14 (70.00) | 4 (57.14) | 0.65 |
| Height z-score | −1.41 ± 1.33 | −1.85 ± 0.92 | 0.43 |
| Weight z-score | −1.48 ± 1.04 | −1.49 ± 0.81 | 0.99 |
| BMI z-score | −0.70 ± 0.81 | −0.35 ± 0.93 | 0.36 |
| sBA(μmol/L) | 271.62 ± 236.61 | 250.15 ± 143.19 | 0.82 |
| Bilirubin (total), mg/dL | 4.47 ± 4.13 | 6.67 ± 6.22 | 0.30 |
| CSS | 3.25 ± 1.02 | 3.29 ± 0.76 | 0.93 |
| ItchRO | 2.97 ± 0.55 | 2.68 ± 0.58 | 0.25 |

All data are mean ± SD unless otherwise indicated. p-value is for the comparison of baseline characteristics according to treatment response status.
BMI, body mass index; CSS, Clinician Scratch Score; dL, deciliters; ItchRO, Itch-Reported Outcome; L, liters; mg, milligrams; sBA, serum bile acid; SD, standard deviation; μmol, micromoles.

Numerically, responders had improved HRQoL measures compared with non-responders across all scales (Table 2). The change in Multidimensional Fatigue Total Scale Score from baseline to week 48 was significantly higher in responders compared with non-responders; Table 2). No clinically meaningful change was observed from baseline to week 48 across all scales in non-responders.

The present multivariate regression analysis demonstrated that compared with baseline, ItchRO treatment response was associated with a clinically meaningful improvement in HRQoL at week 48. Results were strongest for the FI score. Controlling for baseline FI score, responders' scores increased an average of 17 points over the 48 weeks (p<0.05), compared with non-responders. Smaller and non-statistically significant point estimates were observed for the PedsQL and MF scores. Of the 19 HRQoL items selected for individual analysis, six sleep-related items saw significantly larger changes from baseline to week 48 in responders compared with non-responders: trouble sleeping (p=0.001), feeling tired (p=0.030), sleeping a lot (p=0.014), difficulty sleeping through the night (p=0.003), feeling tired upon waking (p<0.001), and taking a lot of naps (p=0.020).

TABLE 2
Mean HRQoL by treatment response status

| | | PedsQL Generic | Multidimensional | Family Impact |
|---|---|---|---|---|
| | | Core Scale | Fatigue Scale | Scale |
| | | N = 27 | N = 21 | N = 26 |
| HRQoL at Baseline | Responders | 58.8 ± 17.9 | 47.3 ± 22.4 | 56.7 ± 18.9 |
| (mean ± SD) | Non-Responders | 61.2 ± 15.1 | 67.4 ± 20.9 | 50.8 ± 18.5 |
| | P-value | 0.75 | 0.12 | 0.48 |
| HRQoL at Week 48 | Responders | 70.4 ± 15.7 | 76.2 ± 15.1 | 73.9 ± 19.6 |
| (mean ± SD) | Non-Responders | 62.4 ± 14.5 | 64.2 ± 15.1 | 54.7 ± 20.0 |
| | P-value | 0.25 | 0.17 | 0.037 |
| HRQoL change from | Responders | 11.6 ± 20.3 | 25.8 ±23.0 | 17.8 ± 23.4 |
| baseline to Week 48 | Non-Responders | 1.2 ± 11.1 | −3.1 ± 19.8 | 3.9 ± 7.8 |
| (mean ± SD) | P-value | 0.21 | 0.032 | 0.14 |

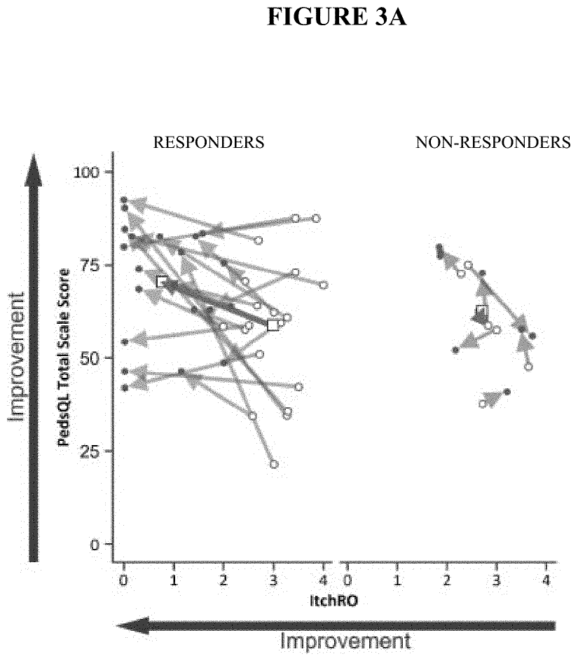

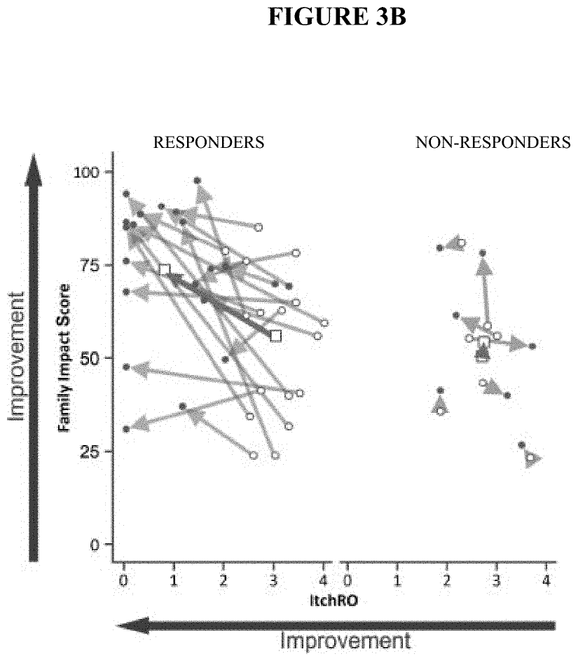

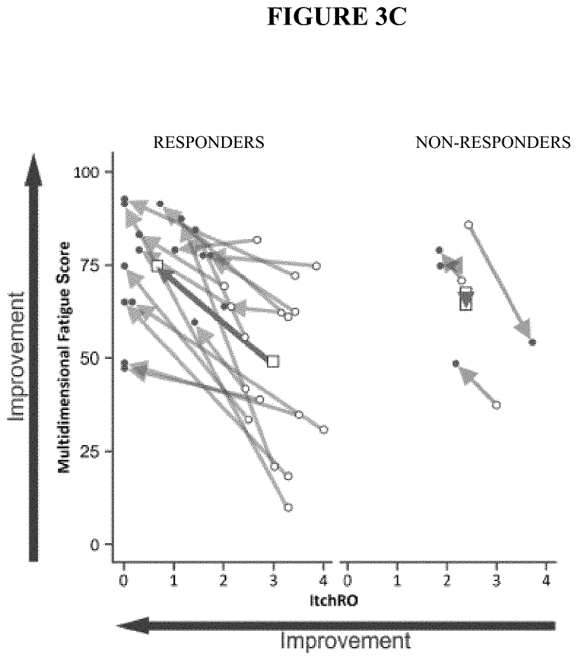

HRQoL scores at baseline and week 48 according to ItchRO response status are shown in FIG. 3. PedsQL Generic Core Total Scale Score (FIG. 3A), Family Impact Total Scale Score (FIG. 3B) and Multidimensional Fatigue Total Scale Score (FIG. 3C) all show that ItchRO treatment response at week 48 was consistently associated with clinically meaningful improvements in all measures of HRQoL. Multivariate regression analysis demonstrated that ItchRO treatment response was associated with a clinically meaningful improvement for all three HRQoL measures from baseline to week 48.

Responders' Family Impact Scale scores increased an average of 16.9 points, more than three times the MCID, over the 48 weeks compared with non-responders (p=0.05) (Table 3). Responders' PedsQL Generic Core Total Scale Score increased on average by 8.8 points (p=0.19), almost two times the MCID, compared with non-responders. Similarly, for the Multidimensional Fatigue score, responders had an average total score increase of 13.9 points (p=0.11), more than two times the MCID, compared with non-responders (Table 3). Results remained robust even after controlling for demographic and clinical characteristics. Of the 19 HRQoLitems selected for individual analysis, six sleep-related items demonstrated significantly larger changes from baseline to week 48 in responders compared with non-responders (Table 4).

TABLE 3
Multivariate regression models of ItchRO treatment response at week 48
versus PedsQL Generic Core Total Scale Score, Family Impact Total Scale
Score, and Multidimensional Fatigue Total Scale Score at week 48.

| | PedsQL Generic | | Family | | Multidimensional | |
|---|---|---|---|---|---|---|
| | Core Scale | | Impact Scale | | Fatigue Scale | |
| | (N = 27) | | (N = 26) | | (N = 21) | |
| | AIC = 226.34 | | AIC = 229.26 | | AIC = 175.86 | |
| Effect | Beta | p-value | Beta | p-value | Beta | p-value |
| Intercept | 8.82 | 0.15 | 4.31 | 0.54 | 11.03 | 0.16 |
| | (−2.64; 20.27) | | (−9.12; 17.74) | | (−3.80; 25.87) | |
| ItchRO | 8.76 | 0.19 | 16.85 | 0.05 | 13.92 | 0.11 |
| treatment | (−3.86; 21.38) | | (1.01; 32.68) | | (−2.49; 30.32) | |
| response at | | | | | | |
| week 48 | | | | | | |
| Yes vs No | | | | | | |
| HRQoL score, | −0.68 | <0.001 | −0.56 | 0.01 | −0.82 | <0.001 |
| baseline, | (−1.01; −0.35) | | (−0.94; −0.17) | | (−1.11; −0.52) | |
| centered at 50 | | | | | | |

One patient was missing Family Impact Scale scores at baseline or week 48 and six patients were missing Multidimensional Fatigue Scale scores at baseline or week 48 and were not included in the models.
AIC, Akaike information Criterion; HRQoL, health-related quality of life; ItchRO, Itch-Reported Outcome.

TABLE 4
Difference between responders and non-responders in change
from baseline to week 48 in selected HRQoL items.

| | Difference in change | p- |
|---|---|---|
| HRQoL item | from baseline to week 48 | value |
| Trouble sleeping | 45.4 | <0.01 |
| Feeling fired | 40.1 | 0.03 |
| Steeping a lot | 55.2 | 0.01 |
| Difficulty steeping through the night | 52.9 | <0.01 |
| Feeling tired upon waking | 72.4 | <0.001 |
| Taking a lot of naps | 40.4 | 0.02 |

In conclusion, patients with ALGS who experienced a pruritus response while receiving maralixibat treatment, on average, achieved greater improvements in HRQoL from baseline to week 48, versus pruritus non-responders. Changes in the Family Impact Scale were statistically significant and clinically meaningful using multivariate regression analysis. Improvements in the PedsQL Generic Core Scale were almost two times the MCID. Multidimensional Fatigue Scale changes were more than two times the MCID.

Significant improvements in six sleep-related items of the HRQoL scales seen in pruritus responders versus non-responders warrant further investigation into the relationship between response to maralixibat and improvements in sleep disturbance.

These data demonstrate that the significant improvements in pruritus seen with maralixibat at week 48 of the ICONIC study are clinically meaningful and are associated with improvements in patients' quality of life.

### Example 3. Predictors of 6-Year Event-Free Survival in Patients with Alagille Syndrome Treated with Maralixibat, an IBAT Inhibitor

Refractory pruritus and liver disease progression are indications for liver transplantation (LTx) in patients with Alagille syndrome (ALGS). Predictors of long-term event-free survival (EFS) and transplant-free survival (TFS) were examined, in ALGS patients enrolled in 3 clinical trials of maralixibat (MRX), an ileal bile acid transporter (IBAT) inhibitor, with up to 6 yrs of follow-up.

MRX-treated ALGS patients from 3 long-term clinical trials were followed for development of clinically significant events (LTx, surgical biliary diversion [SBD], hepatic decompensation [ascites requiring therapy and variceal bleeding], and death) for up to 6 yrs. TFS (LTx and death only) was also assessed. Those who were on MRX 48 weeks from the first dose and had lab results at 48 weeks were included in this analysis. Variables considered in the model included: liver biochemistries, platelets, pruritus (as assessed by ItchRO(Obs) 0¬4 scale), total serum bile acids (sBA), and age. Goodness of fit was assessed using Harrell's concordance statistic (C-statistic). Cutoffs were determined via a grid search. P-values are from a log-rank test.

Patients (N=76) with ALGS aged 14-207 months with moderate-to-severe pruritus were included in this analysis. Maralixibat-treated patients with ALGS from three long-term clinical trials (ClinicalTrials.gov ID: NCT02047318; ClinicalTrials.gov ID: NCT02160782; ClinicalTrials.gov ID: NCT02117713) were followed for development of first clinically significant event (LT, surgical biliary diversion, hepatic decompensation [ascites requiring therapy and variceal bleeding], or death) for up to six years. TFS (LT and death only) was also assessed. Patients who were on maralixibat 48 weeks from the first dose and had lab results at 48 weeks were included in this analysis.

Variables considered in the model included: liver biochemistries, platelets, pruritus (as assessed by Itch-Reported Outcome [Observer] [ItchRO(Obs)] 0-4 scale), total sBA, and age at initiation. These were explored at baseline, week 48, and change from baseline to week 48. Goodness of fit was assessed using Harrell's concordance statistic (C-statistic). Variables with a value ≥0.7 (indicating a good model) were selected for further analysis. Cutoffs for each variable were determined via a grid search across the range of values. Statistical comparisons between the cutoff groups were calculated using a log-rank test.

This analysis included 76 maralixibat-treated patients, with a median follow-up of 266 weeks (range: 53-380). Patient baseline characteristics are shown in Table 5.

TABLE 5
Baseline characteristics in maralixibat-treated patients with ALGS

| Patients (N = 76) | Median (IQR) | |
|---|---|---|
| Age (months) | 70 | (33-126) |
| Male, n (%) | 45 | (59) |
| Total bilirubin (mg/dL) | 2.3 | (0.9-8.4) |
| sBA (μmol/L) | 184 | (78-361) |
| ItchRO(Obs) score | 2.7 | (2.1-3.1) |
| ALT (U/L) | 134 | (95-193) |
| GGT (U/L) | 392 | (188-751) |

ALT, alanine transaminase;
GGT, gamma-glutamyl transferase;
ItchRO(Obs), itch-reported outcome (observer);
IQR, interquartile range;
sBA, serum bile acid

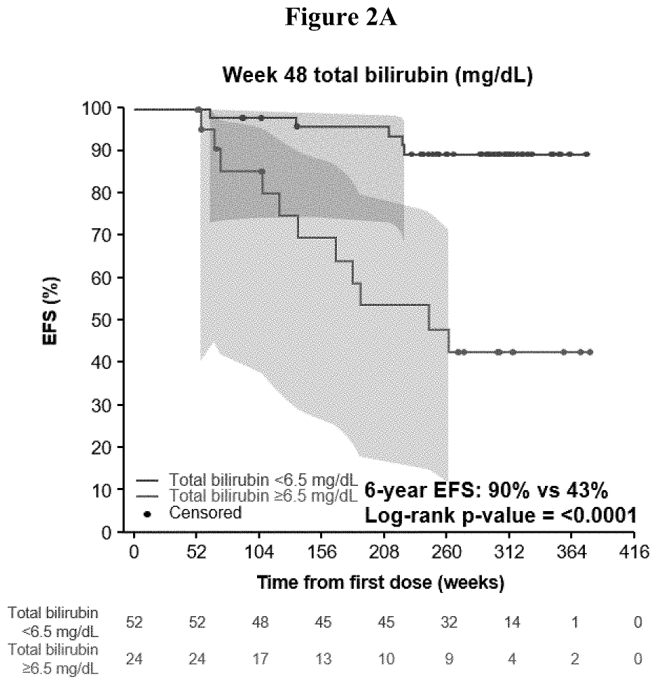

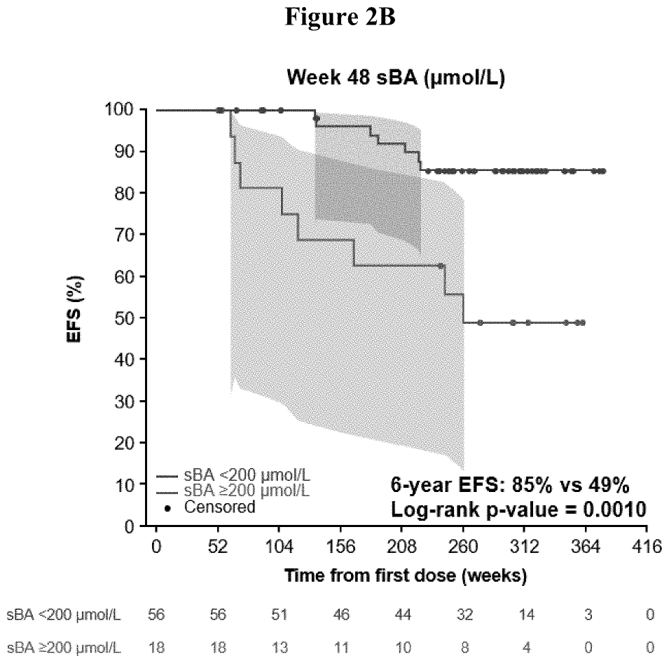

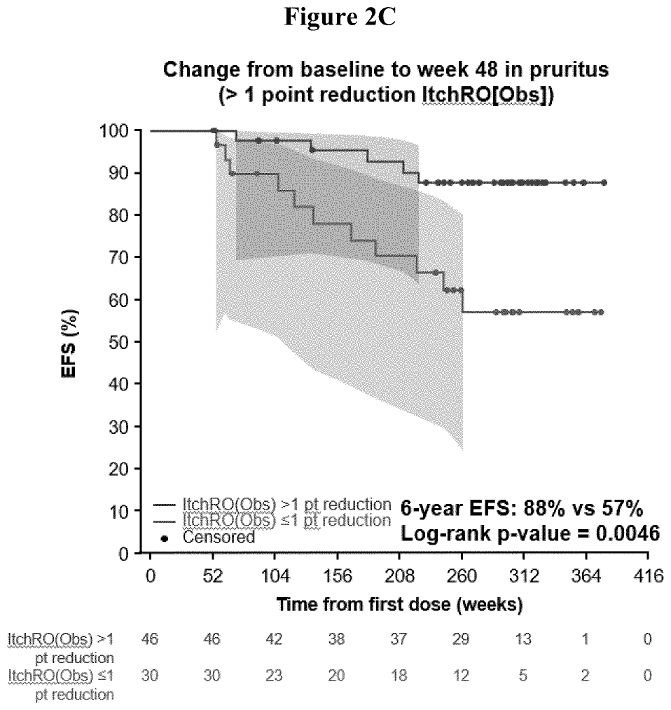

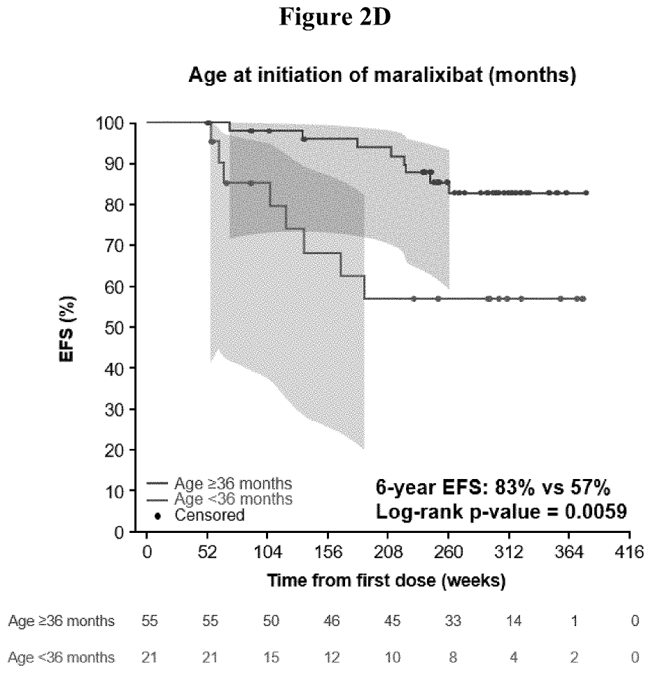

Sixty out of 76 patients remained event-free at the time of analysis. Sixteen patients experienced clinical events: LT (n=10), decompensation (n=3), death (n=2), and surgical biliary diversion (n=1). Variables that were predictive of EFS included: week 48 total bilirubin, week 48 sBA, change from baseline to week 48 in pruritus (ItchRO[Obs]), and age at initiation of maralixibat (Table 6; FIG. 2).

TABLE 6
Predictors of EFS and TFS in maralixibat-
treated patients with ALGS.

| Variable | Better EFS/TFS | Worse EFS/TFS | p-value |
|---|---|---|---|
| Week 48 total bilirubin | <6.5 mg/dL | ≥6.5 mg/dL | |
| | n = 52 | n = 24 | |
| C-statistic: 0.82 | 6-year EFS: 90% | 6-year EFS: 43% | <0.0001 |
| C-statistic: 0.85 | 6-year TFS: 94% | 6-year TFS: 42% | <0.0001 |
| Week 48 sBA | <200 μmol/L | ≥200 μmol/L | |
| | n = 56 | n = 18 | |
| C-statistic: 0.74 | 6-year EFS: 85% | 6-year EFS: 49% | 0.0010 |
| C-statistic: 0.79 | 6-year TFS: 90% | 6-year TFS: 49% | 0.0001 |
| Change from baseline to | >1 pt reduction | ≤1 pt reduction | |
| week 48 ItchRO(Obs) | n = 46 | n = 30 | |
| C-statistic: 0.70 | 6-year EFS: 88% | 6-year EFS: 57% | 0.0046 |
| C-statistic: 0.77 | 6-year TFS: 93% | 6-year TFS: 57% | 0.0007 |
| Age at initiation of | ≥36 months | <36 months | |
| maralixibat | n = 55 | n = 21 | |
| C-statistic: 0.72 | 6-year EFS: 83% | 6-year EFS: 57% | 0.0059 |
| C-statistic: 0.74 | 6-year TFS: 87% | 6-year TFS: 57% | 0.0016 |

ALGS, Alagille syndrome; C-statistic, Harrell's concordance statistic; EFS, event-free survival; ItchRO(Obs), itch-reported outcome (observer); pt, point; sBA, serum bile acid; TFS, transplant-free survival.

The four variables identified as predictors of EFS had high C-statistics over time, indicating that these cutoffs were stable predictors for 2-5 additional years after 48 weeks of maralixibat treatment. These four variables and cutoffs were similarly predictive for TFS.

Fifty-nine (79.7%) patients had ≤2 predictors of worse EFS at the end of 48 weeks of maralixibat treatment (Table 7). The rate of 6-year EFS was 88% in this group. Fifteen (20.3%) patients had ≥3 predictors of worse EFS. The rate of 6-year EFS was 31% in this group.

TABLE 7
Distribution of participants across worse (bold)
and better (underlined) EFS predictor variables.

| Week 48 | | Age at | | | | | |
|---|---|---|---|---|---|---|---|
| total | Week 48 | initiation of | Week 48 | | | 6-year | Number of |
| bilirubin | sBA | maralixibat | Change from BL | | Participants | EFS | EFS |
| (mg/dL) | (μmol/L) | (months) | ItchRO(Obs) | | (N = 74) | (%) | variables |
| <6.5 | <200 | ≥36 | >1 | pt reduction | 30 | 89 | 0 |
| <6.5 | <200 | ≥36 | ≤1 | pt reduction | 9 | 89 | 1 |
| ≥6.5 | <200 | ≥36 | >1 | pt reduction | 5 | | |
| <6.5 | <200 | <36 | >1 | pt reduction | 5 | | |
| <6.5 | ≥200 | ≥36 | >1 | pt reduction | 2 | | |
| ≥6.5 | ≥200 | ≥36 | >1 | pt reduction | 4 | 86 | 2 |
| <6.5 | <200 | <36 | ≤1 | pt reduction | 3 | | |
| <6.5 | ≥200 | ≥36 | ≤1 | pt reduction | 1 | | |
| ≥6.5 | <200 | <36 | ≤1 | pt reduction | 4 | 29 | 3 |
| ≥6.5 | ≥200 | ≥36 | ≤1 | pt reduction | 3 | | |
| <6.5 | ≥200 | <36 | ≤1 | pt reduction | 1 | | |
| ≥6.5 | ≥200 | <36 | ≤1 | pt reduction | 7 | 33 | 4 |

ALGS, Alagille syndrome; BL, baseline; EFS, event-free survival; ItchRO, itch-reported outcome; pt, point; sBA, serum bile acid.

In patients with ALGS, predictors of EFS with maralixibat treatment include (FIGS. 2A-2D): total bilirubin and sBA (both at week 48); pruritus reduction (from baseline to week 48); and age at initiation of maralixibat. As pruritus is often an indication for liver transplant (LT) in patients with ALGS, the present data demonstrated that improvements in pruritus with maralixibat are significantly associated with improved event-free survival (EFS) and transplant-free survival (TFS). The present data identified potential prognostic markers that may better inform patient/provider discussions of clinical outcomes in patients receiving maralixibat treatment.

### Example 4. Serum Bile Acid (sBA) Control in Long-Term Maralixibat-Treated Patients is Associated with Native Liver Survival in Children with Progressive Familial Intrahepatic Cholestasis (PFIC) Due to Bile Salt Export Pump (BSEP) Deficiency

Children with progressive familial intrahepatic cholestasis (PFIC) due to bile salt export pump (BSEP) deficiency present with debilitating pruritus, short stature, and progressive liver disease. The NAPPED study (NCT03930810) shows that ˜50% of patients receive a liver transplant by 10 years of age, but those who achieve serum bile acid (sBA) levels of <100 μmol/L after partial external biliary diversion show improved native liver survival and reductions in aspartate aminotransferase (AST), alanine aminotransferase (ALT), and bilirubin.

Maralixibat (MRX) is an ileal bile acid transport (IBAT) inhibitor approved for the treatment of pruritus in patients with Alagille syndrome aged 1 year and older and under evaluation in patients with PFIC. MRX interrupts the enterohepatic recirculation, reducing pruritus and cholestasis, and improving growth at Week 72 in an open-label, long-term study (INDIGO; NCT02057718). This example describes >4.5 years of treatment of PFIC patients with MRX.

MRX was dosed at 280 μg/kg daily for 48 weeks, increasing to 280 μg/kg twice daily in the extension. The NAPPED sBA threshold (≤100 μmol/L) was applied to patients remaining on-study >4.5 years. Transaminases, bilirubin, and growth were assessed to Week 237.

Of the enrolled patients (n=19) with nontruncated BSEP mutation (median age 4.1 years, range 1-13; 32% male), 7 achieved sBA control and remained on-study as of Week 237. Mean (SE) sBA reduction was 234.4 (80.5) μmol/L (p<0.05) with a mean value of 44.2 (38.8) μmol/L (vs. 299.6 μmol/L at baseline [BL]). Mean reductions in AST and ALT were 35.4 (11.5) and 41.1 (14.3) U/L with mean values of 26.7 (vs. 62.1 at BL) and 16.7 (vs. 57.9 at BL), respectively (both p<0.05). Mean reductions in total and direct bilirubin were 0.1 (0.3) and 0.4 (0.2) mg/dL with mean values of 0.7 (vs. 0.8 at BL) and 0.1 (vs. 0.6 at BL), respectively (p=0.8 and 0.13). Those with abnormal bilirubin, normalized. No ongoing patients were listed for liver transplant after >4.5 years of MRX. Growth (height z-score; p<0.01) and pruritus (p<0.001) improved significantly. Long-term MRX was safe and well tolerated; the most frequent treatment-emergent adverse events were mild to moderate in severity.

Patients who achieved control of sBA during MRX treatment had native liver survival beyond 4.5 years, improved liver biochemistry, and improved growth, suggesting the disease-modifying potential of MRX. These data support MRX as a potential alternative to surgery for children with PFIC due to non-truncating BSEP deficiency.

### Example 5. Maralixibat Treatment Response is Associated with Improved Health-Related Quality of Life in Patients with BSEP Deficiency

Bile salt export pump (BSEP) deficiency is the most common genetic cause of progressive familial intrahepatic cholestasis (PFIC). The disease negatively impacts patients' health-related quality of life (HRQoL). Maralixibat (MRX) is an oral, minimally-absorbed inhibitor of the ileal bile acid transporter (IBAT) recently approved for the treatment of pruritus in patients with Alagille syndrome aged 1 year and older and under evaluation in patients with PFIC. This study assessed the impact of MRX treatment response on HRQoL among patients with BSEP deficiency.

This retrospective analysis included data from patients with BSEP deficiency from the INDIGO Phase 2 open-label trial of MRX in children with PFIC. Pruritus was measured using a validated caregiver-reported Itch Reported Outcome (ItchRO) severity assessment tool (0=none to 4=very severe). Serum bile acid (sBA) response with MRX was defined as a >75% decrease from baseline or reduction to <102 μmol/L from baseline to week 48. HRQoL was assessed using the Pediatric Quality of Life Inventory Generic Core (PedsQL), Family Impact (FI), and Multidimensional Fatigue (MF) scale scores, and were collected via caregiver. The minimal clinically important difference (MCID) for the HRQoL assessments is 5 points. A subset of individual items from the HRQoL scales were also selected for assessment with treatment response. HRQoL was assessed at baseline and week 48 and scores were stratified by treatment response. P-values were calculated using t-test or ANOVA. Multivariate linear regression models were used to assess the relationship between mean change from baseline in HRQoL score and indicators for treatment response, adjusting for baseline HRQoL.

22 patients from the INDIGO trial with PFIC2 had HRQoL data available at week 48, and were included in this analysis (patient baseline characteristics are shown in Table 8, below).

TABLE 8
Patient baseline characteristics in maralixibat
responders and non-responders.

| | sBA treatment response at week 48*^{†} | | |
|---|---|---|---|
| | Responders | Non-responders | |
| | (N = 6)^{‡} | (N = 15) | p-value |
| Age (years) | 4.5 ± 3.2 | 5.0 ± 3.6 | 0.77 |
| Male, n (%) | 2 (33.3) | 4 (26.7) | 1.00 |
| Height z-score | −1.2 ± 0.7 | −1.3 ± 1.0 | 0.73 |
| Weight z-score | −0.6 ± 0.9 | −0.6 ± 0.9 | 0.90 |
| sBA (μmol/L) | 281.6 ± 212.6 | 390.2 ± 108.9 | 0.13 |
| PedsQL Generic | 58.9 ± 8.3 | 66.7 ± 14.9 | 0.25 |
| Core Total Scale Score | | | |
| PedsQL Family | 52.0 ± 13.4 | 65.2 ± 15.4 | 0.09 |
| Impact Total Score | | | |
| PedsQL Multidimensional | 42.5 ± 11.7 | 68.5 ± 22.1 | 0.03 |
| Fatigue Scale Score | | | |
| ItchRO(Obs) | 2.5 ± 0.6 | 2.1 ± 0.9 | 0.33 |

*One patient was lost to follow-up at week 48 and was therefore not evaluate for sBA treatment response;
^{†}18 patients had non-truncating BSEP mutations, and 4 patients had truncating BSEP mutations;
^{‡}Six responders all had non-truncating BSEP mutations.
All data are mean ± SD unless otherwise indicated. p-value is for the comparison of baseline characteristics according to treatment response status.
ItchRO(Obs), Itch-Reported Outcome (Observer); PedsQL, Pediatric Quality of Life; sBA, serum bile acid; SD, standard deviation; μmol/L, micromoles/liter.

At 48 weeks, 6 patients (28.6%) had achieved a treatment response; 1 patient's week 48 sBA response status was unknown and was therefore excluded; 15 participants did not meet the criteria for sBA response and were categorized as non-responders. Baseline HRQoL was lower among responders than non-responders. Other baseline characteristics were similar between the two groups.

The mean HRQoL scores by treatment report status are presented in Table 9, below.

TABLE 9
HRQoL at baseline, week 48, and change from baseline to week 48,
for sBA responders and non-responders to maralixibat treatment

| | | PedsQL Genetic | Multidimesional | Family Impact |
|---|---|---|---|---|
| | | Core Scale | Fatigue Scale | Scale |
| | | (N = 18)* | (N = 16)* | (N = 20)* |
| HRQoL scare at | Responders | 59.0 ± 9.3 | 40.3 ± 12.3 | 52.0 ± 13.4 |
| baseline | Non-responders | 66.8 ± 15.5 | 68.5 ± 22.1 | 65.2 ± 15.4 |
| | p-value | 0.31 | 0.03 | 0.09 |
| HRQoL score at | Responders | 79.3 ± 9.5 | 76.0 ± 13.4 | 60.0 ± 15.5 |
| week 48 | Non-responders | 65.9 ± 13.8 | 69.2 ± 18.5 | 62.7 ± 16.0 |
| | p-value | \|0.07 | 0.51 | 0.73 |
| HRQoL score | Responders | 20.3 ± 17.7 | 35.8 ± 15.1 | 8.0 ± 20.7 |
| change from | 5-point change, n (%)^{†} | 4 (80.0) | 4 (100) | 4 (66.7) |
| baseline to | 10-point change, n (%)^{†} | 3 (60.0) | 4 (100) | 4 (66.7) |
| week 48 | Non-responders | −0.8 ± 10.9 | 0.7 ± 16.7 | −2.5 ± 9.8 |
| | 5-point change, n (%)^{†} | 2 (15.4) | 3 (25.0) | 1 (7.1) |
| | 10-point change, n (%)^{†} | 2 (15.4) | 2 (16.7) | 1 (7.1) |
| | p-value | 0.01 | <0.01 | 0.13 |

*Of the 21 patients included in the sample, 18 (85.7%), 16 (76.2%), and 20 (95.2%) had available data on PedsQL Generic Core Total Scale Score, Multidimensional Fatigue Total Scale Score, and Family Impact Total Scale Score, respectively, at week 48;
^{†}Proportions provided for 5-and 10-point changes were calculated among the number of patients with non-missing week 48 HRQoL data. All data are mean ± SD unless otherwise stated. HRQoL, health-related quality of life; PedsQL, PediatricQuality of Life; sBA, serum bile acid; SD, standard deviation.

Twenty-two patients with BSEP deficiency were included with a mean±SD baseline age of 4.7±3.4 years, sBA of 359.18±148.76 μmol/L, ItchRO(Obs) score of 2.20±0.86, PedsQL score of 64.34±13.54, FI score of 61.22±15.75 and MF score of 60.87±22.78; 31.8% were male. At 48 weeks, 6 patients had achieved a treatment response and baseline HRQoL was lower among responders than non-responders. Responders experienced a greater change from baseline to week 48 across all HRQoL measures versus non-responders (Table). Multivariate regression analyses found sBA response at week 48 was associated with a clinically meaningful improvement in HRQoL from baseline to week 48. Controlling for baseline score, sBA responders' PedsQL scores increased by a mean of 17 points over the 48 weeks versus non-responders (p=0.012) and MF scale scores increased by an average of 22 points versus non-responders (p=0.037). Non-significant differences were observed for the FI score. Five of the ten pre-selected individual HRQoL items, largely related to sleep, demonstrated significant changes from baseline to week 48 in sBA responders versus non-responders: feeling tired during the day (p=0.032), worried about how my child's illness is affecting other family members (p=0.004), difficulty sleeping through the night (p=0.002), feeling tired when he/she wakes up in the morning (p=0.005), and taking a lot of naps (p=0.004).

Responders experienced an improvement from baseline to week 48 across all HRQoL measures compared with non-responders. Statistically significant differences between responders and non-responders were observed in PedsQL Generic Core Score and Multidimensional Fatigue Scores (Table 9, above). The change in Family Impact Score from baseline to week 48 for responders was clinically meaningful, at >1.5 times the MCID, but the difference between responders and non-responders was not statistically significant.

Controlling for baseline PedsQL Generic Core Total Scale Score, multivariate regression analyses found that silk responders' scores increased by a mean of 17 points, more than three times the MCID, over the 48 weeks compared with non-responders (p<0.05) (Table 10). Similarly, for the Multidimensional Fatigue Scale, responders' total scores increased by an average of 22 points compared with non-responders (p<0.05). Smaller and non-statistically significant differences were observed for the Family Impact Total Scale Score.

Five of the 10 individual items in the PedsQL Family Impact Scale, demonstrated significant changes from baseline to week 48 in sBA responders compared with nonresponders: feeling tired during the day (p=0.03); worried about how my child's illness is affecting other family members (p<0.01); difficulty sleeping through the night (p<0.01); feeling tired upon waking (p<0.01); and taking a lot of naps (p<0.01).

TABLE 10
Multivariate regression models of sBA treatment
response at week 48 vs HRQoL scores at week 48.

| | PedsQL Generic | | Multidimensional | | Family Impact | |
|---|---|---|---|---|---|---|
| | Core Scab | | Fatigue Scab | | Scale | |
| | (N = 18*) | | (N = 16*) | | (N = 20*) | |
| | AIC = 142.6 | | AIC = 134.5 | | AIC = 164.2 | |
| Effect | Beta | p-value | Beta | p-value | Beta | p-value |
| Intercept | 7.6 | 0.11 | 9.1 | 0.11 | 2.9 | 0.55 |
| | (−1.3; 16.4) | | (−1.3; 19.4) | | (−6.3; 12.1) | |
| sBA treatment response at | 17 2 | 0.01 | 22.3 | 0.04 | 5.8 | 0.41 |
| week 48 Yes vs No | (5.3; 29.1) | | (3.5; 41.1) | | (−7.7; 19.4) | |
| PedsQL Total Scale Score | −0.5 | 0.02 | −0.5 | 0.03 | −0.4 | 0.10 |
| (baseline, centered at 50) | (−0.9; −0.1) | | (−0.8; −0.1) | | (−0.8; 0.1) | |

Significant differences were observed in PedsQL Generic Core Total Scale Score and Multidimensional Fatigue Total Scale Scores, with a mean (standard deviation) change in score from baseline to week 48 of 20.3 (17.7) and 35.8 (15.1) for responders, compared with −0.8 (10.9) and 0.7 (16.7) for non-responders; p=0.01 and p<0.01, respectively.

More responders experience a change of ≥5 points in the PedsQL Generic Core Total Scale Score compared with non-responders (80.0%* vs 15.4%; p=0.02). A ≥10-point change in PedsQL Multidimensional Fatigue Total Scale Score was experienced by all responders (100%) and two non-responders (16.7%).*

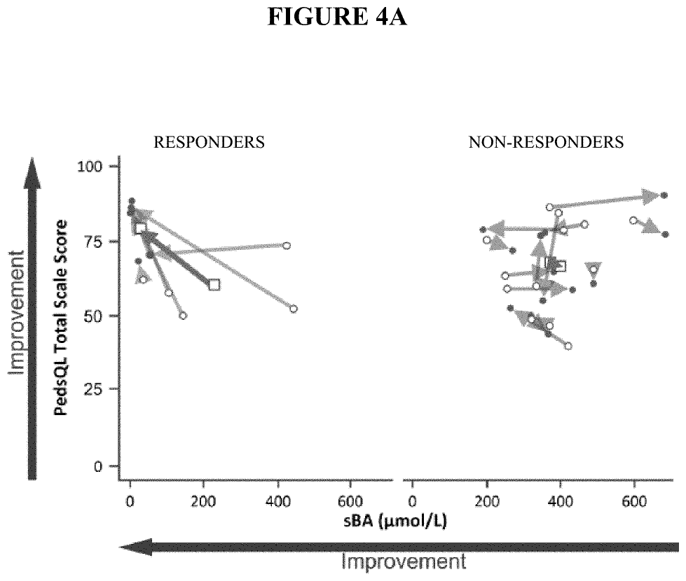

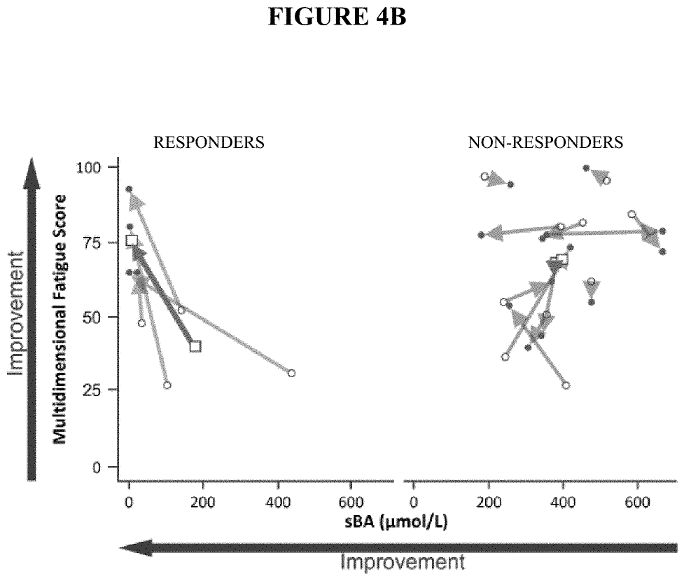

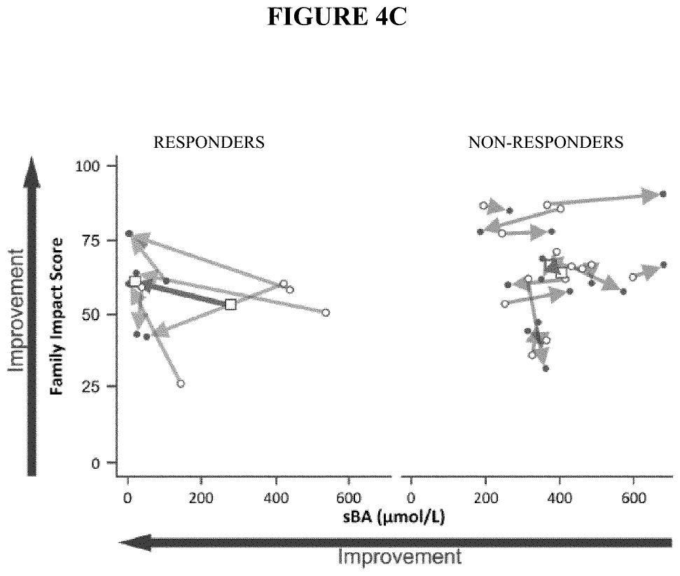

HRQoL scores at baseline and week 48 according to sBAresponse status are shown in FIG. 4. PedsQLGeneric Core Total Scale Score (FIG. 4A), Multidimensional Fatigue Total Scale Score (FIG. 4B), and Family Impact Total Scale Score (FIG. 4C) all show that sBA treatment response at week 48 for individual patients was strongly associated with clinically meaningful improvements in PedsQL Generic Core Total Scale Score and Multidimensional Fatigue Total Scale Score (FIG. 4). *One patient and three patients had missing data for these metrics, respectively.

MRX treatment response at week 48 in patients with BSEP deficiency, as measured by sBA and pruritus, is associated with statistically significant and clinically meaningful improvement in HRQoL across multiple dimensions.

Patients with BSEP deficiency (PFIC2) who responded to maralixibat treatment had statistically significant and clinically meaningful improvements in HRQoL (PedsQL Generic Core Total Scale Score, Multidimensional Fatigue Total Scale Score). Clinically meaningful improvements were also found in the Family Impact Total Scale Score. Statistically significant improvements were also seen in sleep and fatigue measures in patients who were maralixibat responders compared with non-responders.

This analysis demonstrates that maralixibat treatment response at week 48, as measured by sBA, in patients with BSEP deficiency (PFIC2), is associated with a statistically significant and clinically meaningful improvement in HRQoL across multiple dimensions.

### Example 6: Longitudinal Serum Bile Acid Profiling by UHPLC-MS/MS Predicts Cholesteric Pruritus Reduction in Maralixibat Treated Patients with Bile Salt Export Pump Deficiency

Progressive familial intrahepatic cholestasis (PFIC) due to bile salt export pump (BSEP) deficiency is a distressing disease manifested by intractable pruritus, growth delay, and eventually culminating in liver failure. Bile acids have long been implicated in the pathogenesis of pruritus although the exact role they play is unclear. Maralixibat (MRX) is a nonabsorbable apical sodium-dependent bile acid (BA) transport inhibitor that interrupts the enterohepatic circulation of BAs. It was recently FDA approved for the treatment for pediatric cholestasis-associated pruritus because of its ability to reduce the accumulation of circulating and hepatic BAs. Not all patients respond to therapy for reasons that are unclear. The present example describes a targeted metabolomics approach to explore the association between the bile acid metabolome and cholestatic pruritus and for its potential to predict pruritus reduction in response to MRX treatment.

Total and individual serum BAs (sBA) including the major sub-species of TCA, TUDCA, TCDCA, TDCA, TLCA, GCA, GUDCA, GCDCA, GDCA, GLCA, CA, UDCA, CDCA, DCA, LCA and 7α-hydroxy-4-cholesten-3-one (sterol-C4), a surrogate marker for BA synthesis, were measured using a validated ultraperformance liquid chromatography coupled with tandem mass spectrometry (UHPLC-MS/MS) assay. The internal standards, a cocktail of deuterium labeled standards were added to serum, calibrators and QC samples. sBA were extracted and separated on a Kinetex C18 (2.6 μm, 100×3.0 mm) column (Phenomenex, Torrance, Calif.) with gradient elution. Quantification of individual sBA was achieved by multiple reaction monitoring (MRM) of selected transitions. Total C24 bile acid concentrations were calculated from the sum of individual species. These assays were applied to the analysis of serum from 19 patients with genetically confirmed PFIC due to BSEP deficiency (open-label phase 2 INDIGO trial; NCT02057718) treated with MRX. Changes in the composition of sBA associated with pruritus improvement measured with the Itch Reported Outcome Observer (ItchRO[Obs]) score were monitored at intervals over 72 weeks, with pruritus responders, defined as >1 scale reduction, compared with nonresponders. Linear mixed model framework was also used to model longitudinal profiles of sBA and pruritus reduction over time.

Of the 19 patients, 11 met the pruritus-response criteria, which correlated with a reduction from baseline in total sBA that was significantly greater than in nonresponders (p<0.05) in the longitudinal data. Changes were also observed in the individual BA subspecies. Reductions in glycine and taurine-conjugated cholic acid (TCA) associated with pruritus reduction after MRX treatment and correlated with percentage ItchRO(Obs) score reduction (Pearson correlation coefficient: 0.58, 0.50, 0.53 for TCA, glycocholic acid, and total cholic acid [CA], respectively). A trend towards increased proportion of unconjugated BA in responders (9.84±4.87%) was observed compared with nonresponders (0.61±0.24%, p=0.09). More importantly, longitudinal sBA profile changes, e.g. GUDCA, GCDCA, TCDCA significantly correlated with pruritus reduction (χ²=13.35, P<0.001; χ²=12.86, P<0.001; and χ²=19.50, P<0.001, respectively). Concomitant increases in serum sterol-C4 were observed in pruritus responders (χ²=4.70, P<0.05), consistent with the biological action of MRX in blocking the intestinal reabsorption of bile acids.

Targeted analysis of the bile acid metabolome by mass spectrometry revealed significant changes associated with pruritus response to MRX therapy in children with BSEP deficiency and measures of serum sterol-C4 further confirmed efficacy of the drug. These findings offer further interpretation on BA subspecies profile associated with reductions in cholestasis and pruritus, reinforcing data demonstrating that MRX is an effective pharmacological therapy for BSEP deficiency.

### Example 7: Pruritus Intensity is Associated with Cholestasis Biomarkers and Quality of Life Measures after Maralixibat Treatment in Children with Alagille Syndrome

So far, absolute values of pruritus intensity and cholestasis biomarkers have been shown to poorly correlate in children with ALGS. This study evaluates how change in pruritus intensity correlates with change in cholestasis biomarkers in children with ALGS receiving maralixibat (ICONIC study; NCT02160782, described above).

The goal of the present study is to characterize correlations between pruritus, as measured by the Itch-Reported Outcome (ItchRO) Observer tool, and multiple parameters, including sBA and sBA subspecies, autotaxin (ATX), and quality of life measures following maralixibat treatment in children with ALGS.

Twenty-nine of the 31 enrolled participants completed 48 weeks of treatment, with 27 evaluated for this analysis. Baseline characteristics for the analysis population are shown in Table 11.

TABLE 11
Baseline characteristics for the analysis population.

| | | Mean (SD) | | Mean (SD) |
|---|---|---|---|---|
| All | Mean (SD) | CSS | Mean (SD) | ItchRO |
| participants | age | score* | sBA^{†} | score^{†} |
| (N) | (years) | (points) | (μmol/L) | (points) |
| 27 | 5.7 (4.30) | 3.3 (0.94) | 2 [symbol] (213. [symbol] ) | 2. [symbol] (0.5 [symbol] ) |

*CSS score range 0-4; [symbol]
of sBA [symbol] ; [symbol]
ItchRO score range 0-4
CSS, Clinician Scratch Score; ItchRO, Itch-Reported Outcome; L, liter; sBA, serum bile acid; SD, standard deviation, μmol, micromoles [symbol]
indicates data missing or illegible when filed

At week 48, statistically significant correlations with the ItchRO score included CSS score, sBA, growth (height z-score), and ATX, with a trend toward significance with PedsQL™ Family Impact Total Scale (PedsQL™ Impact) scores, as shown in Table 11.

Taurocholic acid (TCA) and glycocholic acid (GCA) also exhibited significant correlations with pruritus in patients with ALGS (Table 11). A statistically significant correlation between the ItchRO and PedsQL™ Multidimensional Fatigue Scale (PedsQL™ Fatigue) scores was also noted as a change from baseline to week 48 (r=−0.59, p=0.0053; Table 12).

TABLE 12
Spearman's rank correlation coefficient data exhibiting
associations between ItchRO score and key parameters.

| | Patients (N) | [symbol] | p-value |
|---|---|---|---|
| Parameters correlated with | | | |
| ItchRO score at week 48 | | | |
| CSS | 27 | 0.64 | 0.0003 |
| TCA | 27 | 0.60 | 0.0010 |
| GCA | 27 | 0.53 | 0.0047 |
| SBA | 27 | 0.47 | 0.0123 |
| Height z-score | 27 | −0.44 | 0.0209 |
| ATX | 27 | 0.44 | 0.0213 |
| PedsQL ™ Impact | 26 | −0.38 | 0.0574 |
| Parameters correlated with | | | |
| ItchRO score as a change | | | |
| from baseline to week 48 | | | |
| PedsQL ™ Fatigue | 21 | −0.59 | 0.0053 |

ATX [symbol] CSS, Clinician Scratch Score; [symbol] , Itch-Reported Outcome; [symbol] . [symbol]
indicates data missing or illegible when filed

The overall average ItchRO score reduction was 1.6 points at week 48. Increasing proportional sBA reductions after 50% appears to be associated with greater ItchRO score reductions (Table 13, below). One participant normalized with an ItchRO score reduction of −3.5 points.

TABLE 13
Change in pruritus intensity in relation to changes in sBA.

| sBA reduction (%) | 50 | 60 | 70 | 80 | 90 |
|---|---|---|---|---|---|
| Patients (N) | 8 | 8 | 6 | 3 | 1 |
| Change in ItchRO score (points) | −1.90 | −2.12 | −2.31 | −2.79 | −2.71 |

ItchRO, Itch-Reported Outcome; sBA, serum bile acid.

Maralixibat treatment in study participants with ALGS led to significant and clinically meaningful improvements in pruritus, using ItchRO and CSS scores. sBA reductions correlated with reductions in pruritus intensity, further supporting the causal relationship between the two. Significant correlations were also found with ATX and height z-score, with a trend toward significance with the PedsQL™ Impact scores. Pruritus was significantly correlated with PedsQL™ Fatigue scores when assessing change from baseline to week 48, suggesting that sleep is improved with decreased pruritus.

Overall, the positive treatment effects of maralixibat in patients with ALGS demonstrate important correlations with multiple clinically relevant parameters at week 48.

### Example 8: Maralixibat for the Treatment of Progressive Familial Intrahepatic Cholestasis: A Long-Term, Open-Label, Phase 2 Study

Children with progressive familial intrahepatic cholestasis, including bile salt export pump (BSEP) and familial intrahepatic cholestasis-associated protein 1 (FIC1) deficiencies, suffer debilitating cholestatic pruritus that adversely affects growth and quality of life (QoL). Reliance on surgical interventions, including liver transplantation, highlights the unmet therapeutic need. INDIGO was an open-label, Phase 2, international, long-term study to assess the efficacy and safety of maralixibat in children with FIC1 or BSEP deficiencies, conducted at 12 hospitals. Thirty-three patients, 12 months to 18 years of age, were enrolled. Eight had FIC1 deficiency and 25 had BSEP deficiency; six with biallelic, protein truncating mutations (t)-BSEP, 19 with >1 nontruncating mutation (nt)-BSEP. Patients received maralixibat 266 μg/kg maralixibat orally, once-daily, from Baseline to Week 72, with twice-daily dosing permitted from Week 72. Long-term efficacy was determined at Week 240. During the study, serum bile acid (sBA) response (reduction in sBA of >75% from baseline or concentrations <102.0 μmol/L) was achieved in seven patients with nt-BSEP; six during once-daily dosing and one after switching to twice-daily dosing. sBA responders demonstrated profound reductions in sBA and pruritus, and increases in height, weight, and QoL. All sBA responders remained liver transplant-free after >5 years. No patients with FIC1 deficiency or t-BSEP deficiency met the sBA responder criteria during the study. Maralixibat was generally well-tolerated throughout the study.

Response to maralixibat was dependent on PFIC subtype; 6 of 19 patients with nt-BSEP experienced rapid and sustained reductions in sBA levels. The seven responders survived with native liver at last follow up and experienced clinically significant reductions in pruritus and meaningful improvements in growth and QoL. The results indicate that maralixibat represents a well-tolerated alternative to surgical intervention.

Most forms of PFIC are caused by mutations in transporters expressed in the canalicular membrane of hepatocytes. Two of the major types of PFIC, bile salt export pump (BSEP) or familial intrahepatic cholestasis-associated protein 1 (FIC1) deficiencies, were included in the current study. BSEP deficiency (or PFIC type 2) is caused by mutations in ATP binding cassette subfamily B member 11 (ABCB11). BSEP is the major bile acid transporter from hepatocytes into canaliculi, and BSEP deficiency is the most common PFIC type. The majority of patients with BSEP deficiency have at least one nonprotein truncating mutation (nt-BSEP) with the potential for some residual BSEP function but about 15% have two variants predicted to cause protein truncation (truncating BSEP [t-BSEP]), resulting in an absence of BSEP function. FIC1 is encoded by P-type ATPase phospholipid transporting 8B1 (ATP8B1), a lipid transporter that is expressed in multiple epithelia, including the canalicular membrane. Deficiency of FIC1 (or PFIC type 1), is associated with extrahepatic manifestations, including chronic diarrhea, pancreatic insufficiency, renal tubular dysfunction, and growth failure.

Here, we present data from INDIGO (Open Label Study of the Efficacy and Long-Term Safety of LUM001 in the Treatment of Cholestatic Liver Disease in Pediatric Patients with Progressive Familial Intrahepatic Cholestasis; LUM001-501; NCT02057718), an open-label, Phase 2 study of the long-term efficacy and safety of maralixibat in children with BSEP or FIC1 deficiencies. INDIGO investigated the effect of maralixibat on sBA levels and cholestatic pruritus, alongside other biochemical markers of cholestasis and liver disease in these patients.

INDIGO was an open-label, Phase 2, international, long-term, multi-center study designed to assess the efficacy and safety of maralixibat (previously known as LUM001-501 or SHP625) in children with FIC1 deficiency or BSEP deficiency. The study was conducted at 12 hospitals in France, Poland, the United Kingdom, and the United States. Screening evaluations were performed in the 6 weeks prior to the start of the study. In patients without documented ATP8B1 or ABCB11 mutations, genetic testing was performed. The study comprised an initial maralixibat dose escalation period, followed by a long-term stable dosing period (up to maralixibat 266 μg/kg given orally, once daily [equivalent to maralixibat chloride 280 μg/kg, and hereafter referred to as ‘266 μg/kg’]). An amendment to the study protocol permitted subsequent dose increases up to 266 μg/kg twice daily if predefined sBA and pruritus benefits were not achieved by Week 72, as well as entry into the long-term extension period of the study.

The primary efficacy endpoint was the change in mean fasting sBA levels from Baseline to Week 13 in the overall intent-to-treat (ITT) study population. The key secondary efficacy endpoint was the mean change in observer-rated pruritus (Itch Reported Outcome Observer [ItchRO(Obs)]) score from Baseline to Week 13 in the overall ITT study population. Other secondary efficacy endpoints included the mean changes from Baseline to Week 13 in total cholesterol, low- and high-density lipoprotein cholesterol (LDL-C and HDL-C, respectively), and serum triglyceride levels in the overall ITT study population. Additional efficacy and safety analyses included the change from Baseline to Week 72 and Week 240 in sBA levels, patient height and weight, bile acid synthesis (as determined by the ratio between sBA levels and 7α-hydroxy-4-cholesten-3-one [7α-C4] levels), quality of life as measured by the Pediatric Quality of Life Inventory™ (PedsQL™), mean changes in ALT, aspartate aminotransferase (AST), and bilirubin (total and direct), lipid profiles, and FSV levels. The ItchRO assessment was not performed at Week 72, so data from Week 48 were used instead.

Efficacy and safety analyses were performed for the overall ITT study population as well as the two PFIC subtypes (FIC1 deficiency and BSEP deficiency) separately. Patients with BSEP deficiency were analyzed overall and according to their mutation type (t-BSEP and nt-BSEP). Long-term changes in pruritus scores, patient height, blood lipids, liver parameters, and transplant-free survival were assessed according to a responder analysis. Analyses were made at Week 72 (due to a natural delineation between the two main periods of the study) and Week 240 (chosen to provide long-term data while minimizing the effects of low patient numbers and missing data observed at later time points).

Quantitative analysis of the 15 major sBAs and serum 7α-C4 was performed at a single site (Cincinnati) by stable isotope dilution electrospray ionization liquid chromatography-mass spectrometry (LCMS/MS) using a fully validated proprietary assay that complies with College of American Pathologists/Clinical Laboratory Improvement Amendments certification. The study protocol defined a composite response to maralixibat as achieving a ≥1.0-point reduction in ItchRO(Obs) score and a ≥70% reduction or normalization of sBA levels from Baseline. Following the publication reporting improved transplant-free survival in individuals who achieved a reduction of sBA >75% from baseline or concentrations of <102.0 μmol/L after surgical interruption of the enterohepatic circulation, thresholds were changed to use this new definition in the post-hoc responder analyses for this study. Regardless of definition, the number of responders remained the same. Pruritus was measured using the ItchRO tool, which was specifically developed and validated to evaluate pruritus in children with cholestatic liver diseases, based on a five-point scale where 0=‘none’ and 4=‘very severe’. This tool has been shown to detect clinically relevant changes in cholestatic pruritus in children across all ages, with changes ≥1.0 point being clinically meaningful. The ItchRO measure was completed twice daily via an eDiary. Parents/caregivers completed the ItchRO(Obs) assessment for all patients. Additionally, children ≥9 years of age completed the ItchRO(Pt) assessment. Levels of ALT, AST, bilirubin (total and direct), lipids, and FSV were assessed using standard clinical laboratory techniques. Quality of life was assessed using the PedsQL™ questionnaire. The minimum clinically important difference for child-reported and parent/caregiver-reported scores was a change of 4.4 or 4.5 points in Total Scale Score, respectively. The occurrence of treatment-emergent adverse events (TEAEs) and serious adverse events (SAEs) was assessed throughout the study.

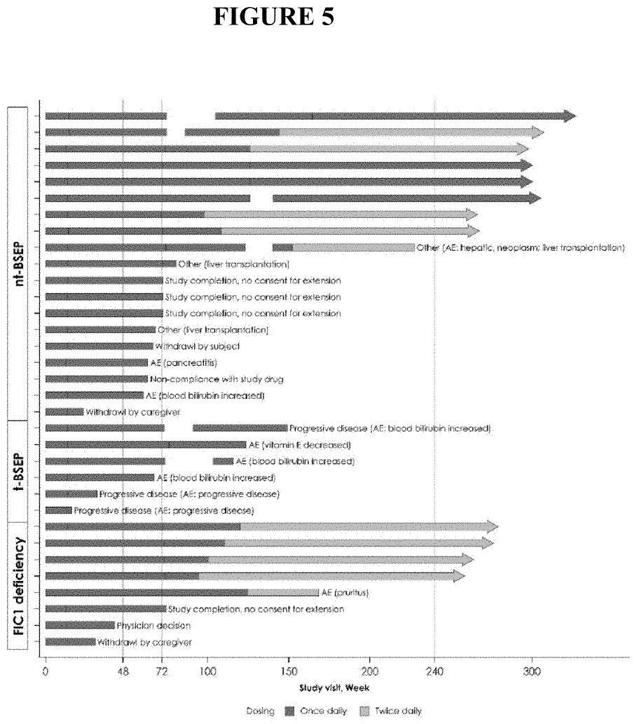

Thirty-seven patients were screened between February 2014 and July 2015. Three patients had low sBA (sBA<3×ULN of 8.5 μmol/L) and 1 participant had elevated INR and these were not included in the study. Thirty-three patients were enrolled; a total of eight (24%) had FIC1 deficiency and 25 (76%) had BSEP deficiency (six with t-BSEP, 19 with nt-BSEP; FIG. 5). Of the 33 patients enrolled: all completed study treatment up to Week 13; 22 (67%) completed study treatment up to Week 72 (six with FIC1 deficiency, 16 with BSEP deficiency); and 11 (33%) discontinued. Eighteen patients consented to remain on study treatment following Week 72; six patients discontinued during this period (FIG. 5). The mean study week at which dose escalation to 266 μg/kg twice daily occurred was Week 110 (range Weeks 94-152). At end of study (Week 240), a total of 12 patients had each received >4 years of maralixibat treatment.

Efficacy results for both short term (13 weeks) and long term (up to 72 weeks) are described below. Notable differences in short- and long-term efficacy results were observed between the disease subtypes, with patients with nt-BSEP deficiency demonstrating significant improvements in various parameters (shown below).

Within the overall ITT study population (all 33 enrolled patients; PFIC1 and PFIC2 combined), there was no significant decrease in sBA levels from Baseline to Week 13 (−16.9 μmol/L [95% confidence intervals (CIs) −74.830, 41.070; P=0.884]). However, pruritus significantly improved by Week 13 in the ITT population, with a mean reduction from Baseline in ItchRO(Obs) score of −0.8 (95% CI −1.04, −0.53; P<0.001) and ItchRO(Pt) score of −1.0 (95% CI −1.55, −0.47; P=0.002).

Patients with BSEP deficiency demonstrated predictably varied responses to treatment according to disease type, with patients with nt-BSEP (n=19) showing the greatest response. During the study, sBA response (reduction in sBA of >75% from baseline or concentrations <102.0 μmol/L) was achieved by a subset of seven patients (37%) with nt-BSEP (hereafter referred to as ‘sBA responders’). These sBA responders demonstrated profound and sustained sBA reductions, as well as reductions in pruritus and other parameters. Six achieved response while receiving 266 μg/kg once daily and one achieved response while receiving 266 μg/kg twice-daily dosing after Week 97. No patient with t-BSEP met the sBA responder criteria at any point in the study and all patients with t-BSEP discontinued before Week 240. The following results summarize the long-term efficacy for all patients with BSEP deficiency and summarize data for sBA responders versus sBA non-responders.

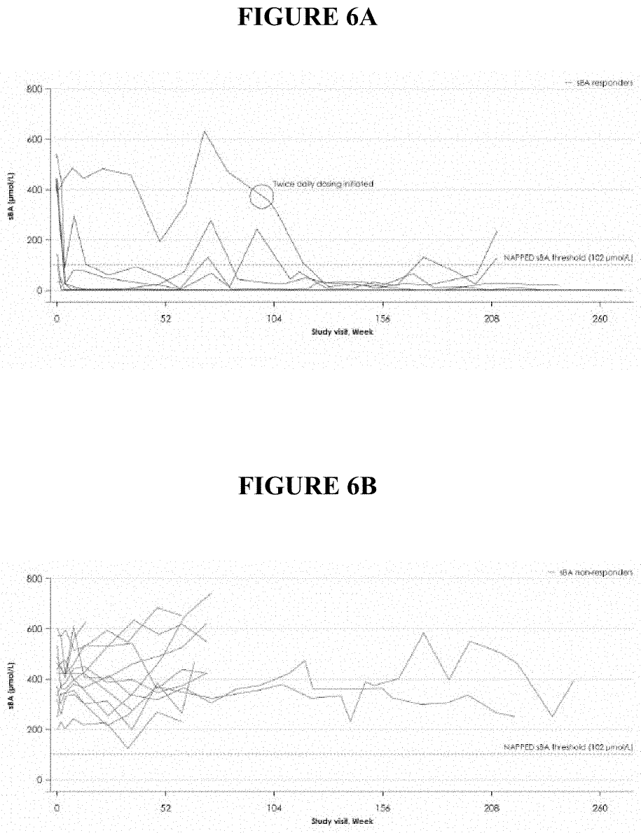

Baseline serum ALT, AST, total and direct bilirubin, cholesterol, and triglycerides were all significantly lower in responders than in non-responders. Changes in mean sBA levels are shown in FIG. 6. Compared with Baseline, sBA levels within sBA responders decreased rapidly upon treatment initiation (within Weeks 2-4) with mostly durable responses maintained long-term throughout the study (FIG. 6A). This pattern of response was not observed in sBA non-responders (FIG. 6A).

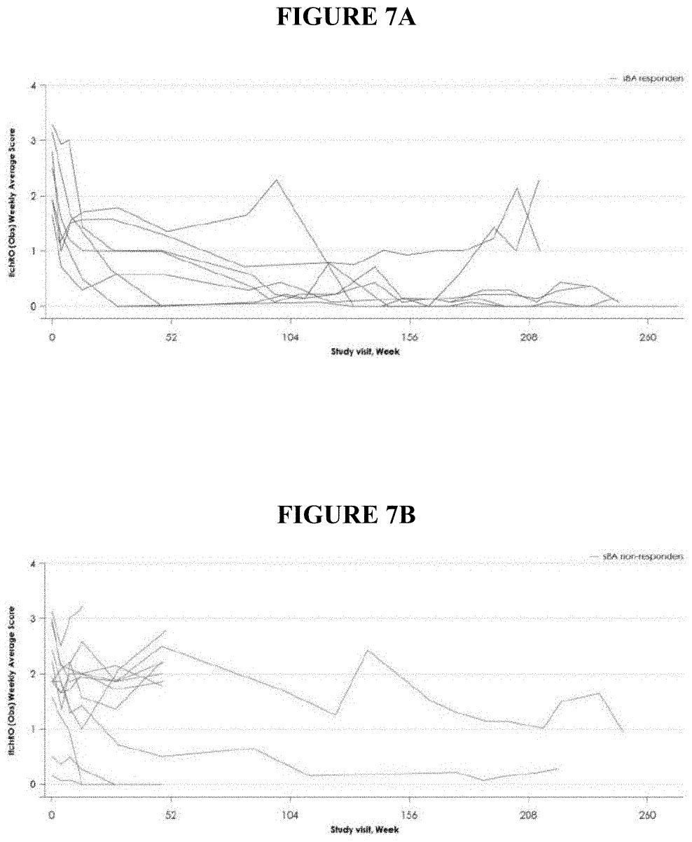

Changes in mean ItchRO(Obs) scores are shown in FIG. 7. All seven sBA responders experienced a >1.0-point clinically meaningful reduction in ItchRO(Obs) scores after achieving sBA response (FIG. 7A), and an additional three sBA non-responders achieved clinically meaningful reductions in ItchRO(Obs) scores (Supporting FIG. 7B).

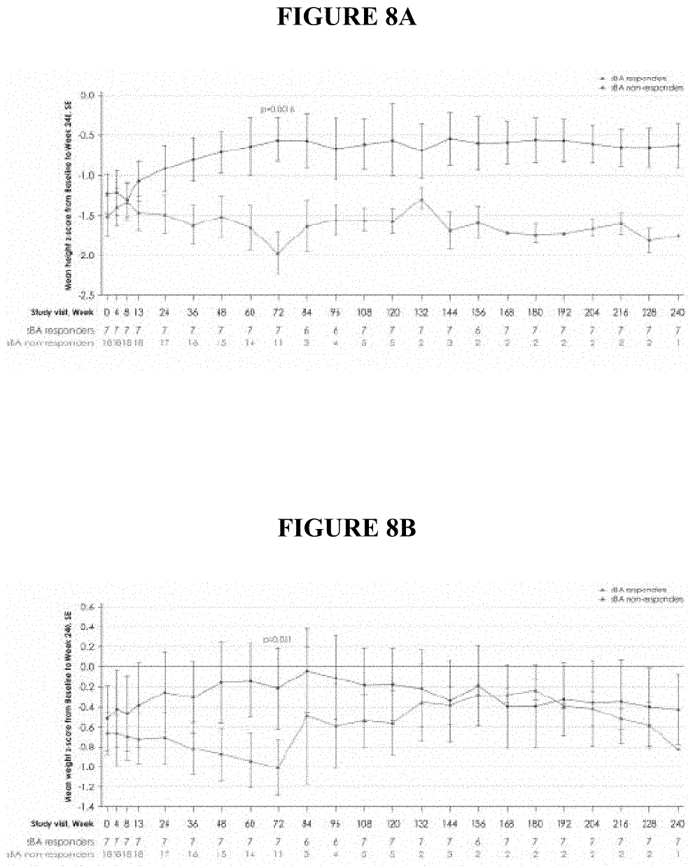

Changes in mean height and weight z-scores are shown in FIG. 8. sBA responders experienced a gradual increase from Baseline in mean height and weight z-scores to Week 72 (0.67, 95% CI 0.369, 0.976; P=0.0016 and 0.30, 95% CI −0.001, 0.603; P=0.051, respectively, which was maintained to Week 240; FIGS. 8A and 8B). This growth benefit contrasted with a decrease in height and weight z-scores in sBA non-responders when assessed at Week 72 (0.49, 95% CI −0.948, −0.041; P<0.001 versus sBA responders and −0.30, 95% CI −0.606, −0.012; P=0.013 versus sBA responders, respectively).

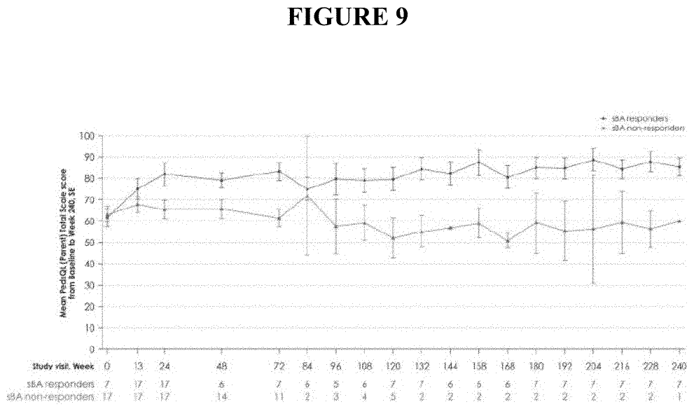

Changes in mean PedsQL™ scores are shown in FIG. 9. Clinically meaningful improvements in mean PedsQL™ score were observed within sBA responders upon treatment initiation, remaining consistent throughout the study (a change from Baseline to Week 240 of 24 points, 95% CI 6.7, 40.6; P=0.014, respectively; FIG. 9).

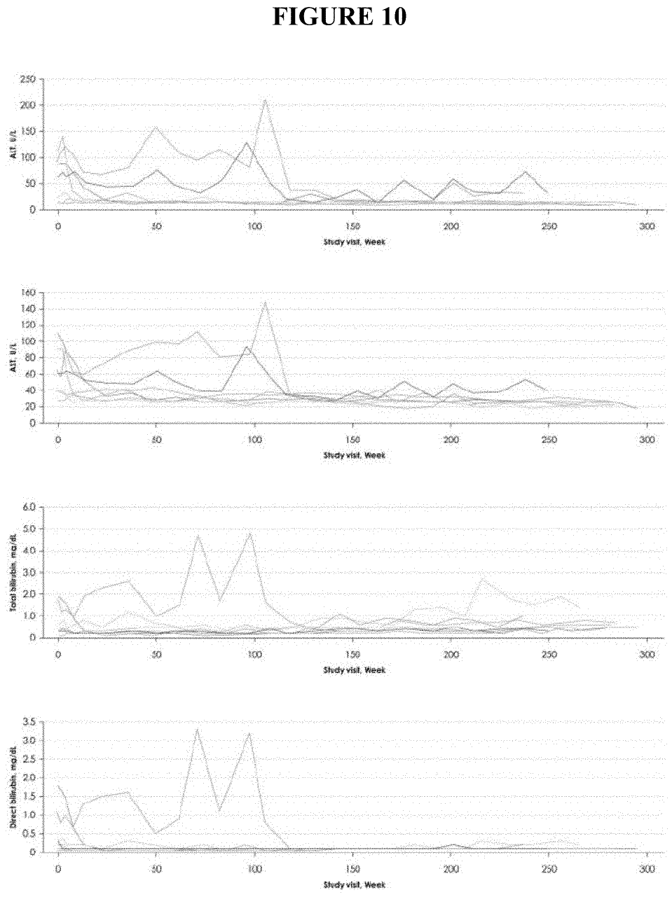

Biochemical assessments are shown in FIG. 10. sBA responders experienced improvements and/or normalization of serum ALT, AST, and bilirubin levels (if elevated at Baseline; FIG. 10). Upon treatment with maralixibat, the mean level of serum 7α-C4 increased in patients with nt-BSEP, gradually increasing throughout the study, reaching statistical significance at Week 240 (+26.29 ng/mL, 95% CI 4.07, 48.50; P=0.027).

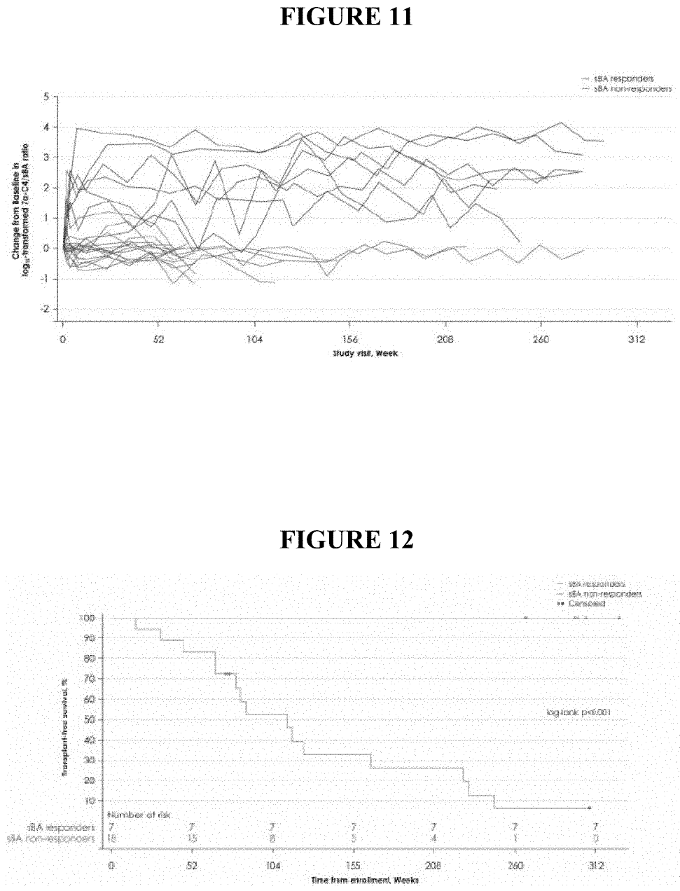

Evidence of biological response following treatment can be assessed by looking for increased bile acid synthesis, reflected by increases in serum 7α-C4. When combined with a reduction in sBA, the 7α-C4/sBA ratio, compared to Baseline, becomes a sensitive marker of biological response. The 7α-C4/sBA ratio in patients with BSEP deficiency was generally increased, compared with Baseline, in sBA responders as opposed to sBA non-responders (FIG. 11). Of note, the 7α-C4/sBA ratio in the seventh sBA responder fluctuated during once daily dosing but showed a clear increase upon initiation of twice-daily dosing.

All sBA responders demonstrated transplant-free survival after >5 years of receiving maralixibat compared with none of the sBA non-responders (P<0.001; FIG. 12). Two sBA responders, who were listed for liver transplantation (for the indication of refractory pruritus) at the beginning of the study, improved sufficiently to be removed from the transplant waiting list.

Data describing the additional long-term efficacy analyses within the patients with FIC1 deficiency show that mean levels of sBA, ItchRO(Obs) scores, and PedsQL™ scores did not change significantly from Baseline. No patient with FIC1 deficiency met the sBA responder criteria over the course of the study. Four patients with FIC1 deficiency remain on the study drug, possibly due to receiving some pruritus benefit.

Throughout the study, maralixibat was generally safe and well tolerated (FIG. 5). All patients experienced ≥1 TEAE, with most being mild or moderate in severity (58%) and transient in nature. The most common TEAEs were pyrexia (20 [61%] patients), diarrhea (19 [58%] patients), and cough (18 [55%] patients). Eight patients (one with FIC1 deficiency, seven with BSEP deficiency) discontinued maralixibat due to an adverse event during the study (four due to nonserious events of increases in serum bilirubin; one due to a nonserious event of pancreatitis; one due to a nonserious event of decreased vitamin E; one due to a nonserious event of pruritus; one due to a nonserious event of hepatic mass [reported as a Grade 1 event of liver nodules resulting in the patient subsequently undergoing liver transplantation]). The patient with pancreatitis was reported to have a history of pancreatitis (3 prior episodes), which occurred within a 2-year period before starting maralixibat. A total of 15 patients had serious TEAEs reported during the study, of which abdominal pain, diarrhea, and gastroenteritis were the only SAEs reported in ≥one patient (two patients each). No deaths were reported during the study.

The present study reports the results of up to 5 years of treatment with maralixibat, a minimally absorbed MAT inhibitor with the potential to mimic the effects of surgical biliary diversion, in children with FIC1 deficiency or BSEP deficiency. In this study, the primary endpoint of sBA reduction in the overall ITT study population over the first 13 weeks of treatment was not met; however, importantly, response to treatment was found to be dependent on the different PFIC subtypes. A group of seven patients (37%; 7/19) with nt-BSEP (deemed treatment sBA responders) experienced a sustained, clinically relevant and statistically significant response with improvement in multiple parameters. In addition to reductions in sBA levels, this response included a ≥1.0-point reduction in ItchRO(Obs) score, improvements of serum transaminases and bilirubin, and an improvement in quality of life and growth parameters. Notably, these patients have remained transplant-free and without surgical biliary diversion for over 5 years. These findings are consistent with the NAPPED consortium findings on SBD, which demonstrated that a threshold sBA reduction post-SBD led to similar biochemical and long-term outcomes, improving the natural history benefits.

In contrast to all sBA responders that had at least one nonprotein truncating mutation within ABCB11, none of the six patients with t-BSEP achieved an sBA response. These observations strongly suggest that residual BSEP function is necessary for a maralixibat response. This contention is strongly supported by the differences seen in baseline biochemistry, where lower levels of bilirubin, liver enzymes and lipids were seen in the subsequent responders. These parameters all suggest milder cholestasis and are consistent with greater retained BSEP function. In patients with nt-BSEP there were sBA non-responders. Although these patients may have had insufficient BSEP function, other factors may have determined their lack of response. These factors may include the degree of which Farnesoid X receptor (FXR) regulated bile acid synthesis, levels of IBAT expression, dose of maralixibat, and even small bowel fluid volumes. It is also not immediately obvious why patients with FIC1 deficiency in this study failed to respond to maralixibat. It is possible that the factors above also play a role. Furthermore, the reduced FXR expression that has been observed in these patients, might lead to increased ASBT expression and that would consequently necessitate higher doses of maralixibat in this sub-group.

The 7α-C4/sBA ratio increased rapidly upon initiation of treatment in sBA responders and was sustained throughout the duration of the study, suggesting that this ratio may be a sensitive predictor of response to treatment with maralixibat, better than changes in serum 7α-C4 alone. The seventh sBA responder had fluctuations in their 7α-C4/sBA ratio during once-daily dosing, which clearly increased upon twice-daily dosing thus becoming an sBA responder. Concordantly, a few patients who discontinued at this stage had a 7α-C4/sBA ratio similar to those of sBA responders, suggesting that these patients may have demonstrated a similar response had they received twice-daily dosing. Therefore, 7α-C4/sBA ratio could be used to identify sBA non-responders who would benefit from dose escalation.

Maralixibat was generally well tolerated in patients with PFIC after receiving up to 5 years of treatment, even in patients receiving twice-daily dosing. Although 12 patients discontinued treatment with maralixibat due to adverse events or liver transplantation following disease progression, this was not unexpected, owing to the progressive nature of PFIC. The majority of TEAEs reported during the study were mild-to-moderate, transient, gastrointestinal TEAEs, possibly due to an increased colonic bile acid flux following IBAT inhibition and did not result in treatment discontinuations. These results were in line with the safety profile observed in previous studies of maralixibat.

Treatment with maralixibat did not have any significant detrimental effects on the levels of FSV or liver transaminases. Limitations of this study include the lack of a placebo-controlled element and the relatively small sample size. Considering the rare nature of PFIC, and the dramatic treatment effect on sBA responders who likely avoided life-altering surgery, the findings can be considered as highly relevant and clinically significant. The primary endpoint was based on the response in the whole cohort. However, it is now clear that there was a dichotomous response to treatment, and, as such, a responder analysis would have been more appropriate. Finally, the open-label design limits some conclusions that can be drawn from this study in the absence of a control group, although it would not have been feasible to generate >5 years of placebo-controlled data. The absence of a control group is mitigated by the dramatic and sustained treatment effects seen in the sBA responders, relative to Baseline.

In conclusion, this study demonstrates that maralixibat can lead to rapid and sustained reductions in sBA levels in patients with nt-BSEP leading to transplant-free survival, as well as reductions in pruritus and meaningful improvements in growth and quality of life. These data support the NAPPED findings on use of therapeutic bile acid depletion in nt-BSEP, and the observation that reductions in sBA are a prognostic marker of native liver survival. Maralixibat, therefore, can be considered a realistic and effective treatment strategy, benefiting the lives of patients and caregivers by relieving disease symptoms, increasing transplant-free survival, and providing a well-tolerated, nonsurgical alternative to surgical biliary diversion.

### REFERENCES

Kamath B M, Stein P, Houwen R H J, et al. Potential of ileal bile acid transporter inhibition as a therapeutic target in Alagille syndrome and progressive familial intrahepatic cholestasis. Liver Int 2020; 40:1812-1822.
Vandriel S, Liting L, She H, et al. Clinical features and natural history of 1154 Alagille syndrome patients: results from the international multicenter GALA study group. J Hepatol 2020; 73:S554-S555 (and associated poster presentation).
Wang K S, Tiao G, Bass L M, et al. Analysis of surgical interruption of the enterohepatic circulation as a treatment for pediatric cholestasis. Hepatology 2017; 65:1645-1654.
Shneider B L, Spino C, Kamath B M, et al. Placebo-controlled randomized trial of an intestinal bile salt transport inhibitor for pruritus in Alagille syndrome. Hepatol Commun 2018; 2:1184-1198.
Malatack J J & Doyle D. A drug regimen for progressive familial cholestasis type 2. Pediatrics 2018; 141:e20163877.
Mirum Pharmaceuticals, Inc. LIVMARLI™ (maralixibat) Prescribing Information. 2021. Accessed online at:
https://www.accessdata.fda.gov/drugsatfda_docs/label/2021/214662s0001b1.pdf on Oct. 21, 2021.
Gonzales E, Hardikar W, Stormon M, et al. Maralixibat treatment significantly reduces pruritus and serum bile acids in patients with Alagille syndrome: results from a randomized Phase II study with 4 years of follow-up. Lancet 2021; In press.
ClinicalTrials.gov ID: NCT02047318. Accessed online at: https://clinicaltrials.gov/ct2/show/NCT02047318 on Oct. 21, 2021.
ClinicalTrials.gov ID: NCT02160782. Accessed online at: https://clinicaltrials.gov/ct2/show/NCT02160782 on Oct. 21, 2021.
ClinicalTrials.gov ID: NCT02117713. Accessed online at: https://clinicaltrials.gov/ct2/show/NCT02117713 on Oct. 21, 2021.
Baker A, Kerkar N, Todorova L, Kamath B M, Houwen R H J. Systematic review of progressive familial intrahepatic cholestasis. Clin Res Hepatol Gastroenterol 2019; 43:20-36.
Gunaydin M, Bozkurter Cil A T. Progressive familial intrahepatic cholestasis: diagnosis, management, and treatment. Hepat Med 2018; 10:95-104.
Malatack J J, Doyle D. A drug regimen for progressive familial cholestasis type 2. Pediatrics 2018; 141:e20163877.
Srivastava A. Progressive familial intrahepatic cholestasis. J Clin Exp Hepatol 2014; 4:25-36. van Wessel D B E, Thompson R J, Gonzales E, Jankowska I, Sokal E, Grammatikopoulos T, et al. Genotype correlates with the natural history of severe bile salt export pump deficiency. J Hepatol 2020; 73:84-93.
Davit-Spraul A, Fabre M, Branchereau S, Baussan C, Gonzales E, Stieger B, et al. ATP8B1 and ABCB11 analysis in 62 children with normal gamma-glutamyl transferase progressive familial intrahepatic cholestasis (PFIC): phenotypic differences between PFIC1 and PFIC2 and natural history. Hepatology 2010; 51:1645-1655.
Knisely A S, Strautnieks S S, Meier Y, Stieger B, Byrne J A, Portmann B C, et al. Hepatocellular carcinoma in ten children under five years of age with bile salt export pump deficiency. Hepatology 2006; 44:478-486.
Bergasa N V. Pruritus of cholestasis. In: Carstens E, Akiyama T, eds. Itch: Mechanisms and Treatment. Boca Raton (Fla.): CRC Press/Taylor & Francis, 2014.
Vitale G, Pirillio M, Mantovani V, Marasco E, Aquilano A, Gamal N, et al. Bile salt export pump deficiency disease: two novel, late onset, ABCB11 mutations identified by next generation sequencing. Ann Hepatol 2016; 15:795-800.
Bull L N, Thompson R J. Progressive familial intrahepatic cholestasis. Clin Liver Dis 2018; 22:657-669.
Davit-Spraul A, Gonzales E, Baussan C, Jacquemin E. Progressive familial intrahepatic cholestasis. Orphanet J Rare Dis 2009; 4:1.
Dawson P A. Role of the intestinal bile acid transporters in bile acid and drug disposition. Handb Exp Pharmacol 2011:169-203.
Shneider B L, Spino C, Kamath B M, Magee J C, Bass L M, Setchell K D, et al. Placebo-controlled randomized trial of an intestinal bile salt transport inhibitor for pruritus in Alagille syndrome. Hepatol Commun 2018; 2:1184-1198.
Xiao L, Pan G. An important intestinal transporter that regulates the enterohepatic circulation of bile acids and cholesterol homeostasis: the apical sodium-dependent bile acid transporter (SLC10A2/ASBT). Clin Res Hepatol Gastroenterol 2017; 41:509-515.
Gonzales E, Sturm E, Stormon M, Sokal E, Hardikar W, Lacaille F, et al. Phase 2 placebo-controlled withdrawal study of the ASBT inhibitor maralixibat in children with Alagille syndrome. Presented at International Liver Congress™, Apr. 10-14, 2019, Vienna, Austria.
Gonzales E M, Sturm E, Stormon M, Sokal E M, Hardikar W, Lacaille F, et al. Durability of treatment effect with long-term maralixibat in children with Alagille syndrome: 4-year safety and efficacy results from the ICONIC study. Presented at American Association for the Study of Liver Diseases Annual Meeting®, Nov. 8-12, 2019, Boston, Mass., USA.
Minim Pharmaceuticals. Livmarli™ (maralixibat) oral solution. Prescribing Information, revised September, 2021.
Kamath B M, Abetz-Webb L, Kennedy C, Hepburn B, Gauthier M, Johnson N, et al. Development of a novel tool to assess the impact of itching in pediatric cholestasis. Patient 2018; 11:69-82.
Foster B, Gauthier M, Vig P, Jaecklin T, Kamath B M, Andrae D A. Itch Reported Outcome tool for caregivers of pediatric patients with cholestatic liver disease: an analysis of validation and scoring from the ICONIC maralixibat. Presented at Virtual ISPOR 2020, May 18-20, 2020.
Thompson R J, Jaecklin T J, Vig P. Genotype and dose-dependent response to maralixibat in patients with bile salt export pump deficiency. Presented at 6th World Congress of Pediatric Gastroenterology, Hepatology and Nutrition, Jun. 3-6, 2020, Copenhagen, Denmark.
Varni J W, Burwinkle T M, Seid M, Skarr D. The PedsQL 4.0 as a pediatric population health measure: feasibility, reliability, and validity. Ambul Pediatr 2003; 3:329-341.
Chen F, Ananthanarayanan M, Emre S, Neimark E, Bull L N, Knisely A S, et al. Progressive familial intrahepatic cholestasis, type 1, is associated with decreased farnesoid X receptor activity. Gastroenterology 2004; 126:756-764

All references cited anywhere within this specification are incorporated herein by reference in their entirety for all purposes.

While preferred embodiments of the present invention have been shown and described herein, it will be obvious to those skilled in the art that such embodiments are provided by way of example only.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating cholestatic liver disease in a subject in need thereof, the method comprising administering to the subject a therapeutically effective amount of an ileal bile acid transporter (IBAT) inhibitor, wherein the IBAT inhibitor is selected from the group consisting of 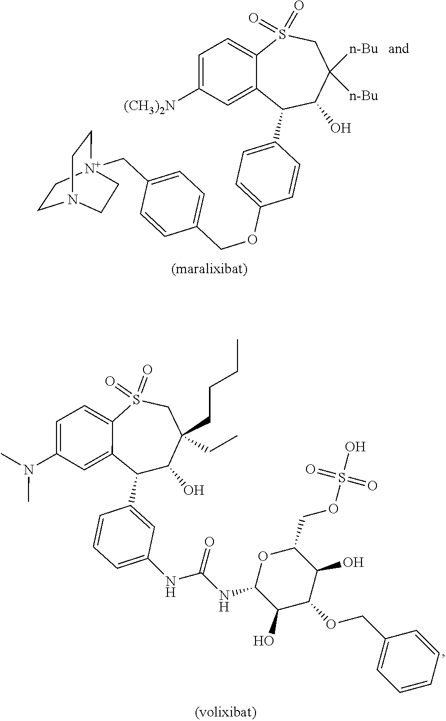
or a pharmaceutically acceptable salt thereof, wherein the treatment increases event-free survival (EFS) of the subject by reducing one or more of:
a) total bilirubin (TB) to about 6.5 mg/dL or below and/or by at least about 1.0 mg/dL;
b) total serum bile acids (sBA) to about 200 μmol/L or below, and
c) pruritus score as measured by an Itch Reported Outcome (ItchRO) and/or ItchRO(Obs) severity assessment tool by at least about 1 point,
wherein the TB, the sBA, or the pruritus score is reduced as compared to the time of first administration of the IBAT inhibitor.

2.-4. (canceled)

5. The method of claim 1, wherein the TB, the sBA, or the pruritus score is determined 18 weeks, or 24 weeks, or 48 weeks after initiation of the IBAT inhibitor treatment.

6.-8. (canceled)

9. A method for providing a prediction of response to an IBAT inhibitor therapy for treatment of cholestatic liver disease in a subject in need thereof by predicting event-free survival (EFS), the method comprising:
obtaining one or more of total bilirubin (TB) data, total serum bile acids (sBA) data, pruritus reduction data and age of the subject at initiation of treatment with the IBAT inhibitor, and
using the data obtained for the subject to predict EFS.

10. The method of claim 9, wherein the EFS is predicted when the TB is less than about 6.5 mg/dL, or when the sBA level after treatment with the IBAT inhibitor is less than about 200 μmol/L, or when the pruritus reduction is at least about 1 point after treatment with the IBAT inhibitor compared to the pruritus at the time of first administration of the IBAT inhibitor, wherein the pruritus is measured by an Itch Reported Outcome (ItchRO) and/or ItchRO(Obs) severity assessment tool.

11. (canceled)

12. (canceled)

13. The method of claim 10, wherein the TB, the sBA level, or the pruritus is determined 18 weeks, or 24 weeks, or 48 weeks after initiation of the IBAT inhibitor treatment.

14.-19. (canceled)

20. The method of claim 9, wherein the EFS is predicted when the age of the subject at the time of initiation of treatment is equal to or higher than about 36 months.

21. The method of claim 1, wherein the EFS comprises survival in the absence of one or more of hepatic decompensation, surgical biliary diversion, liver transplantation or death.

22. The method of claim 21, wherein the EFS comprises survival of the subject in the absence of liver transplant.

23. The method of claim 1, wherein treatment with the IBAT inhibitor further results in reduction of cholestatic pruritus.

24. The method of claim 1, wherein the administration is sufficient to result in event-free survival of the subject for at least 18 months following the first dose of the IBAT inhibitor.

25.-26. (canceled)

27. The method of claim 1, wherein the cholestatic liver disease is a pediatric cholestatic liver disease.

28. The method of claim 1, wherein the cholestatic liver disease is an adult cholestatic liver disease.

29. The method of claim 1, wherein the cholestatic liver disease is non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy, contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome (ALGS), biliary atresia, post-Kasai biliary atresia, post-liver transplantation biliary atresia, post-liver transplantation cholestasis, post-liver transplantation associated liver disease, intestinal failure associated liver disease, bile acid mediated liver injury, MRP2 deficiency syndrome, or neonatal sclerosing cholangitis.

30. The method of claim 27, wherein the cholestatic liver disease is ALGS, PFIC, BRIC, PSC, PBC, or biliary atresia.

31. The method of claim 1, wherein sBA comprise one or more of TCA, TUDCA, TCDCA, TDCA, TLCA, GCA, GUDCA, GCDCA, GDCA, GLCA, CA, UDCA, CDCA, DCA, and LCA.

32. A method of treating cholestatic liver disease with pruritus in a subject in need thereof, the method comprising administering to the subject a therapeutically effective amount of an IBAT inhibitor, wherein the IBAT inhibitor is selected from the group consisting of 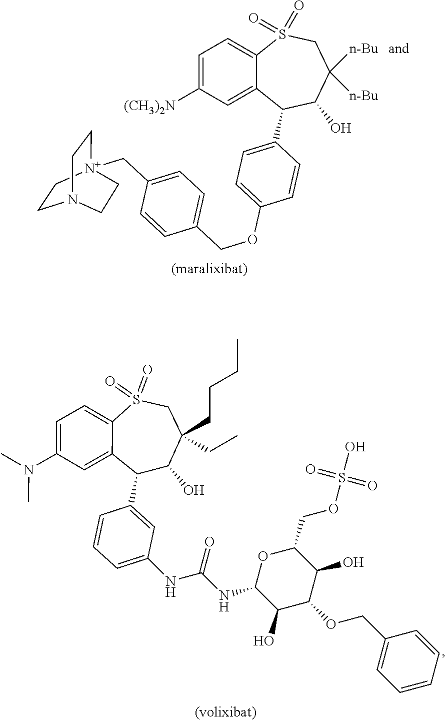
or a pharmaceutically acceptable salt thereof, wherein the administration increases event-free survival (EFS) of the subject for at least 18 months following the first dose of the IBAT inhibitor by reducing pruritus score as measured by an Itch Reported Outcome (ItchRO) and/or ItchRO(Obs) severity assessment tool by at least about 1 point.

33. The method of claim 32, wherein the cholestatic liver disease with pruritus is selected from the group consisting of ALGS, PFIC, BRIC, PSC, PBC, and biliary atresia.

34.-36. (canceled)

37. A method of treating cholestatic liver disease with elevated total serum bile acids (sBA) in a subject in need thereof, the method comprising administering to the subject a therapeutically effective amount of an IBAT inhibitor, wherein the IBAT inhibitor is selected from the group consisting of 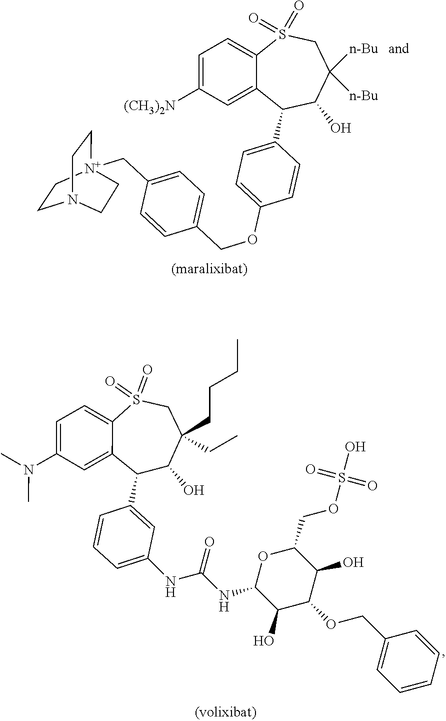
or a pharmaceutically acceptable salt thereof, wherein the administration increases event-free survival (EFS) of the subject for at least 18 months following the first dose of the IBAT inhibitor by reducing sBA to about 200 μmol/L or below.

38. The method of claim 37, wherein the cholestatic liver disease with elevated sBA is selected from the group consisting of ALGS, PFIC, BRIC, PSC, PBC, and biliary atresia.

39.-41. (canceled)

42. The method of claim 37, wherein sBA comprise one or more of TCA, TUDCA, TCDCA, TDCA, TLCA, GCA, GUDCA, GCDCA, GDCA, GLCA, CA, UDCA, CDCA, DCA, and LCA.

43. A method of treating cholestatic liver disease with elevated total bilirubin (TB) in a subject in need thereof, the method comprising administering to the subject a therapeutically effective amount of an IBAT inhibitor, wherein the IBAT inhibitor is selected from the group consisting of 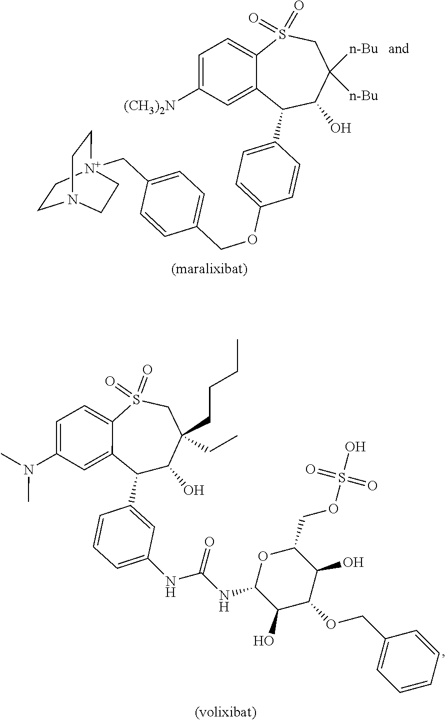
or a pharmaceutically acceptable salt thereof, wherein the administration increases event-free survival (EFS) of the subject for at least 18 months following the first dose of the IBAT inhibitor by reducing TB to about 6.5 mg/dL or below and/or by at least about 1.0 mg/dL.

44. The method of claim 43, wherein the cholestatic liver disease with elevated TB is biliary atresia (BA).

45.-47. (canceled)

48. The method of claim 1, wherein the IBAT inhibitor is administered once daily.

49. (canceled)

50. The method of claim 1, wherein the IBAT inhibitor is administered in an amount of about 0.1 mg to about 100 mg per dose.

51.-53. (canceled)

52. The method of claim 1, wherein the IBAT inhibitor is administered in an amount of about 400 ug/kg/day to about 800 ug/kg/day.

54.-57. (canceled)

58. The method of claim 1, wherein the IBAT inhibitor is

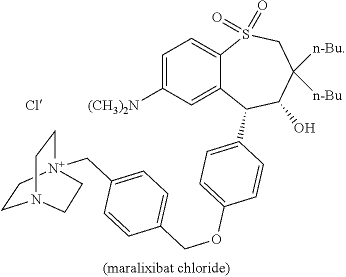

59. The method of claim 1, wherein the IBAT inhibitor is

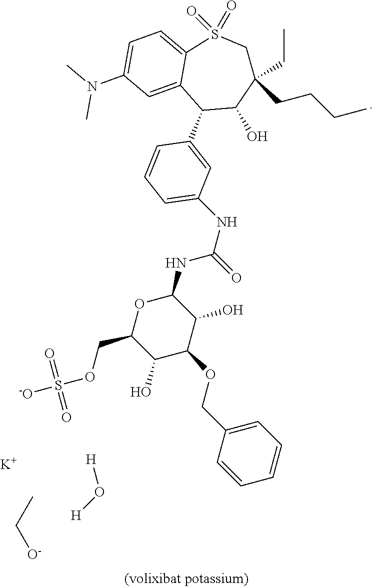

60. The method of claim 1, wherein the subject is a pediatric subject.

61. The method of claim 60, wherein the pediatric subject is 0 to 18 years of age.

62. The method of claim 1, wherein the IBAT inhibitor is administered orally.

63. The method of claim 1, wherein less than 10% of the IBAT inhibitor is systemically absorbed.

64. (canceled)

65. A method of treating Alagille syndrome in a pediatric subject in need thereof, the method comprising administering to the subject a therapeutically effective amount of maralixibat or a pharmaceutically acceptable salt thereof, wherein the administration increases event-free survival (EFS) of the subject for at least 18 months following the first dose of maralixibat by reducing one or more of:
a) total bilirubin (TB) to about 6.5 mg/dL or below and/or by at least about 1.0 mg/dL;
b) total serum bile acids (sBA) to about 200 μmol/L or below, and
c) pruritus score as measured by an Itch Reported Outcome (ItchRO) and/or ItchRO(Obs) severity assessment tool by at least about 1 point.

66. The method of claim 65, wherein the treatment increases liver transplant-free survival (TFS) for at least 18 months following the first dose of maralixibat.

67. A method for providing a prediction of response to maralixibat therapy for treatment of Alagille syndrome in a subject in need thereof by predicting event-free survival (EFS) for 6 years following the first dose of the maralixibat, the method comprising:
obtaining one or more of total bilirubin (TB) data, total serum bile acids (sBA) data, pruritus reduction data and age of the subject at initiation of treatment with maralixibat, and
using the data obtained for the subject to predict the EFS.

68. The method of claim 65, wherein the maralixibat is maralixibat chloride.

69. (canceled)
